(19) United States
(12) Patent Application Publication
(10) Pub. No.: US 2014/0013469 A1
(43) Pub. Date: Jan. 9, 2014

(54) ISOLATED POLYNUCLEOTIDES EXPRESSING OR MODULATING microRNAs OR TARGETS OF SAME, TRANSGENIC PLANTS COMPRISING SAME AND USES THEREOF IN IMPROVING NITROGEN USE EFFICIENCY, ABIOTIC STRESS TOLERANCE, BIOMASS, VIGOR OR YIELD OF A PLANT

(71) Applicants: Rudy Maor, Rechovot (IL); Iris Nesher, Tel Aviv (IL)

(72) Inventors: Rudy Maor, Rechovot (IL); Iris Nesher, Tel Aviv (IL)

(73) Assignee: A.B. SEEDS LTD., Lod (IL)

(21) Appl. No.: 13/881,437

(22) PCT Filed: Oct. 25, 2011
(86) PCT No.: PCT/IB2011/054763

§ 371 Date: Apr. 25, 2013

Related U.S. Application Data
(60) Provisional application No. 61/406,184, filed on Oct. 25, 2010.

(51) Int. Cl. C12N 15/82

(52) U.S. Cl. CPC C12N 15/8216
USPC 800/285; 800/278; 800/290; 800/298

(57) ABSTRACT

Isolated polynucleotides expressing or modulating microRNAs or targets of same are provided. Also provided are transgenic plants comprising same and uses thereof in improving nitrogen use efficiency, abiotic stress tolerance, biomass, vigor or yield of a plant.

### RELATED APPLICATION/S

This application claims priority from U.S. Provisional Patent Application No. 61/406,184 filed on Oct. 25, 2010, the contents of which are hereby incorporated by reference in its entirety.

### FIELD AND BACKGROUND OF THE INVENTION

The present invention, in some embodiments thereof, relates to isolated polynucleotides expressing or modulating microRNAs or targets of same, transgenic plants comprising same and uses thereof in improving nitrogen use efficiency, abiotic stress tolerance, biomass, vigor or yield of a plant.

Plant growth is reliant on a number of basic factors: light, air, water, nutrients, and physical support. All these factors, with the exception of light, are controlled by soil to some extent, which integrates non-living substances (minerals, organic matter, gases and liquids) and living organisms (bacteria, fungi, insects, worms, etc.). The soil's volume is almost equally divided between solids and water/gases. An adequate nutrition in the form of natural as well as synthetic fertilizers, may affect crop yield and quality, and its response to stress factors such as disease and adverse weather. The great importance of fertilizers can best be appreciated when considering the direct increase in crop yields over the last 40 years, and the fact that they account for most of the overhead expense in agriculture. Sixteen natural nutrients are essential for plant growth, three of which, carbon, hydrogen and oxygen, are retrieved from air and water. The soil provides the remaining 13 nutrients.

Nutrients are naturally recycled within a self-sufficient environment, such as a rainforest. However, when grown in a commercial situation, plants consume nutrients for their growth and these nutrients need to be replenished in the system. Several nutrients are consumed by plants in large quantities and are referred to as macronutrients. Three macronutrients are considered the basic building blocks of plant growth, and are provided as main fertilizers; Nitrogen (N), Phosphate (P) and Potassium (K). Yet, only nitrogen needs to be replenished every year since plants only absorb approximately half of the nitrogen fertilizer applied. A proper balance of nutrients is crucial; when too much of an essential nutrient is available, it may become toxic to plant growth. Utilization efficiencies of macronutrients directly correlate with yield and general plant tolerance, and increasing them will benefit the plants themselves and the environment by decreasing seepage to ground water.

Nitrogen is responsible for biosynthesis of amino and nucleic acids, prosthetic groups, plant hormones, plant chemical defenses, etc, and thus is utterly essential for the plant. For this reason, plants store nitrogen throughout their developmental stages, in the specific case of corn during the period of grain germination, mostly in the leaves and stalk. However, due to the low nitrogen use efficiency (NUE) of the main crops (e.g., in the range of only 30-70%), nitrogen supply needs to be replenished at least twice during the growing season. This requirement for fertilizer refill may become the rate-limiting element in plant growth and increase fertilizer expenses for the farmer. Limited land resources combined with rapid population growth will inevitably lead to added increase in fertilizer use. In light of this prediction, advanced, biotechnology-based solutions to allow stable high yields with an added potential to reduce fertilizer costs are highly desirable. Subsequently, developing plants with increased NUE will lower fertilizer input in crop cultivation, and allow growth on lower-quality soils.

The major agricultural crops (corn, rice, wheat, canola and soybean) account for over half of total human caloric intake, giving their yield and quality vast importance. They can be consumed either directly (eating their seeds which are also used as a source of sugars, oils and metabolites), or indirectly (eating meat products raised on processed seeds or forage). Various factors may influence a crop's yield, including but not limited to, quantity and size of the plant organs, plant architecture, vigor (e.g. seedling), growth rate, root development, utilization of water and nutrients (e.g., nitrogen), and stress tolerance. Plant yield may be amplified through multiple approaches; (1) enhancement of innate traits (e.g., dry matter accumulation rate, cellulose/lignin composition), (2) improvement of structural features (e.g., stalk strength, meristem size, plant branching pattern), and (3) amplification of seed yield and quality (e.g., fertilization efficiency, seed development, seed filling or content of oil, starch or protein). Increasing plant yield through any of the above methods would ultimately have many applications in agriculture and additional fields such as in the biotechnology industry.

Two main adverse environmental conditions, malnutrition (nutrient deficiency) and drought, elicit a response in the plant that mainly affects root architecture (Jiang and Huang (2001), Crop Sci 41:1168-1173; Lopez-Bucio et al. (2003), Curr Opin Plant Biol, 6:280-287; Morgan and Condon (1986), Aust J Plant Physiol 13:523-532), causing activation of plant metabolic pathways to maximize water assimilation. Improvement of root architecture, i.e. making branched and longer roots, allows the plant to reach water and nutrient/fertilizer deposits located deeper in the soil by an increase in soil coverage. Root morphogenesis has already shown to increase tolerance to low phosphorus availability in soybean (Miller et al., (2003), Funct Plant Biol 30:973-985) and maize (Zhu and Lynch (2004), Funct Plant Biol 31:949-958). Thus, genes governing enhancement of root architecture may be used to improve NUE and drought tolerance. An example for a gene associated with root developmental changes is ANR1, a putative transcription factor with a role in nitrate (NO3⁻) signaling. When expression of ANR1 is down-regulated, the resulting transgenic lines are defective in their root response to localized supplies of nitrate (Zhang and Forde (1998), Science 270:407). Enhanced root system and/or increased storage capabilities, which are seen in responses to different environmental stresses, are strongly favorable at normal or optimal growing conditions as well.

Abiotic stress refers to a range of suboptimal conditions as water deficit or drought, extreme temperatures and salt levels, and high or low light levels. High or low nutrient level also falls into the category of abiotic stress. The response to any stress may involve both stress specific and common stress pathways (Pastori and Foyer (2002), Plant Physiol, 129: 460-468), and drains energy from the plant, eventually resulting in lowered yield. Thus, distinguishing between the genes activated in each pathway and subsequent manipulation of only specific relevant genes could lead to a partial stress response without the parallel loss in yield. Contrary to the complex polygenic nature of plant traits responsible for adaptations to adverse environmental stresses, information on miRNAs involved in these responses is very limited. The most common approach for crop and horticultural improvements is through cross breeding, which is relatively slow, inefficient, and limited in the degree of variability achieved because it can only manipulate the naturally existing genetic diversity. Taken together with the limited genetic resources (i.e., compatible plant species) for crop improvement, conventional breeding is evidently unfavorable. By creating a pool of genetically modified plants, one broadens the possibilities for producing crops with improved economic or horticultural traits.

### SUMMARY OF THE INVENTION

According to an aspect of some embodiments of the present invention there is provided a method of improving nitrogen use efficiency, abiotic stress tolerance, biomass, vigor or yield of a plant, the method comprising expressing within the plant an exogenous polynucleotide having a nucleic acid sequence at least 90% identical to SEQ ID NOs: 10, 6-9, 21, 22, 23-37, 38-52, 1209, 1211, 1212, wherein said nucleic acid sequence is capable of regulating nitrogen use efficiency of the plant, thereby improving nitrogen use efficiency, abiotic stress tolerance, biomass, vigor or yield of the plant.

According to an aspect of some embodiments of the present invention there is provided a transgenic plant exogenously expressing a polynucleotide having a nucleic acid sequence at least 90% identical to SEQ ID NOs: 10, 6-9, 23-37, wherein said nucleic acid sequence is capable of regulating nitrogen use efficiency of the plant.

According to some embodiments of the invention, said exogenous polynucleotide encodes a precursor of said nucleic acid sequence.

According to some embodiments of the invention, said precursor of said nucleic acid sequence is at least 60% identical to SEQ ID NO: 21, 22, 38-52, 1209, 1211, 1212.

According to some embodiments of the invention, said exogenous polynucleotide encodes a miRNA or a precursor thereof.

According to some embodiments of the invention, said exogenous polynucleotide encodes a siRNA or a precursor thereof.

According to some embodiments of the invention, said exogenous polynucleotide is selected from the group consisting of SEQ ID NO: 10, 6-9, 21, 22, 23-37, 38-52, 1209, 1211, 1212.

According to an aspect of some embodiments of the present invention there is provided an isolated polynucleotide having a nucleic acid sequence at least 90% identical to SEQ ID NO: 6, 7 and 9, wherein said nucleic acid sequence is capable of regulating nitrogen use efficiency of a plant.

According to some embodiments of the invention, said nucleic acid sequence is selected from the group consisting of SEQ ID NO: 6, 7 and 9.

According to some embodiments of the invention, said polynucleotide encodes a precursor of said nucleic acid sequence.

According to some embodiments of the invention, said polynucleotide encodes a miRNA or a precursor thereof.

According to some embodiments of the invention, said polynucleotide encodes a siRNA or a precursor thereof.

According to an aspect of some embodiments of the present invention there is provided a nucleic acid construct comprising the isolated polynucleotide above under the regulation of a cis-acting regulatory element.

According to some embodiments of the invention, said cis-acting regulatory element comprises a promoter.

According to some embodiments of the invention, said promoter comprises a tissue-specific promoter.

According to some embodiments of the invention, said tissue-specific promoter comprises a root specific promoter.

According to an aspect of some embodiments of the present invention there is provided a method of improving nitrogen use efficiency, abiotic stress tolerance, biomass, vigor or yield of a plant, the method comprising expressing within the plant an exogenous polynucleotide which downregulates an activity or expression of a gene encoding an RNAi molecule having a nucleic acid sequence at least 90% identical to SEQ ID NOs: 4, 1-3,5,57-449, 454-846 and 53-56, 1209, thereby improving nitrogen use efficiency, abiotic stress tolerance, biomass, vigor or yield of a plant.

According to an aspect of some embodiments of the present invention there is provided a transgenic plant exogenously expressing a polynucleotide which downregulates an activity or expression of a gene encoding an RNAi molecule having a nucleic acid sequence at least 90% identical to SEQ ID NOs: 4, 1-3,5,57-449, 454-846 and 53-56, 1209.

According to an aspect of some embodiments of the present invention there is provided an isolated polynucleotide which downregulates an activity or expression of a gene encoding an RNAi molecule having a nucleic acid sequence selected from the group consisting of SEQ ID NOs: 4, 1-3,5,57-449, 454-846 and 53-56, 1209.

According to some embodiments of the invention, said polynucleotide encodes a miRNA-Resistant Target as set forth in SEQ ID N01104-1124.

According to some embodiments of the invention, said isolated polynucleotide encodes a target mimic as set forth in SEQ ID NO: 18 or 19.

According to an aspect of some embodiments of the present invention there is provided a nucleic acid construct comprising the isolated polynucleotide above under the regulation of a cis-acting regulatory element.

According to some embodiments of the invention, said cis-acting regulatory element comprises a promoter.

According to some embodiments of the invention, said promoter comprises a tissue-specific promoter.

According to some embodiments of the invention, said tissue-specific promoter comprises a root specific promoter.

According to some embodiments of the invention, the method further comprises growing the plant under limiting nitrogen conditions.

According to some embodiments of the invention, the method further comprises growing the plant under abiotic stress.

According to some embodiments of the invention, said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

According to some embodiments of the invention, the plant is a monocotyledon.

According to some embodiments of the invention, the plant is a dicotyledon.

According to an aspect of some embodiments of the present invention there is provided a method of improving nitrogen use efficiency, abiotic stress tolerance, biomass, vigor or yield of a plant, the method comprising expressing within the plant an exogenous polynucleotide encoding a polypeptide having an amino acid sequence at least 80% homologous to SEQ ID NOs: 927-1021, wherein said polypeptide is capable of regulating nitrogen use efficiency of the plant, thereby improving nitrogen use efficiency, abiotic stress tolerance, biomass, vigor or yield of the plant.

According to an aspect of some embodiments of the present invention there is provided a transgenic plant exogenously expressing a polynucleotide encoding a polypeptide having an amino acid sequence at least 80% homologous to SEQ ID NOs: 927-1021, wherein said polypeptide is capable of regulating nitrogen use efficiency of the plant.

According to an aspect of some embodiments of the present invention there is provided a nucleic acid construct comprising a polynucleotide encoding a polypeptide having an amino acid sequence at least 80% homologous to SEQ ID NOs: 927-1021, wherein said polypeptide is capable of regulating nitrogen use efficiency of the plant, and wherein said polynucleotide is under a transcriptional control of a cis-acting regulatory element.

According to some embodiments of the invention, said polynucleotide is selected from the group consisting of SEQ ID NO: 1022-1090.

According to some embodiments of the invention, said polypeptide is selected from the group consisting of SEQ ID NO: 927-1021.

According to some embodiments of the invention, said cis-acting regulatory element comprises a promoter.

According to some embodiments of the invention, said promoter comprises a tissue-specific promoter.

According to some embodiments of the invention, said tissue-specific promoter comprises a root specific promoter.

According to some embodiments of the invention, the method further comprises growing the plant under limiting nitrogen conditions.

According to some embodiments of the invention, the method further comprises growing the plant under abiotic stress.

According to some embodiments of the invention, said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

According to some embodiments of the invention, the plant is a monocotyledon.

According to some embodiments of the invention, the plant is a dicotyledon.

According to an aspect of some embodiments of the present invention there is provided a method of improving nitrogen use efficiency, abiotic stress tolerance, biomass, vigor or yield of a plant, the method comprising expressing within the plant an exogenous polynucleotide which downregulates an activity or expression of a polypeptide having an amino acid sequence at least 80% homologous to SEQ ID NOs: 854-894, wherein said polypeptide is capable of regulating nitrogen use efficiency of the plant, thereby improving nitrogen use efficiency, abiotic stress tolerance, biomass, vigor or yield of the plant.

According to an aspect of some embodiments of the present invention there is provided a transgenic plant exogenously expressing a polynucleotide which downregulates an activity or expression of a polypeptide having an amino acid sequence at least 80% homologous to SEQ ID NOs: 854-894, wherein said polypeptide is capable of regulating nitrogen use efficiency of the plant.

According to an aspect of some embodiments of the present invention there is provided a nucleic acid construct comprising a polynucleotide which downregulates an activity or expression of a polypeptide having an amino acid sequence at least 80% homologous to SEQ ID NOs: 854-894, wherein said polypeptide is capable of regulating nitrogen use efficiency of a plant, said nucleic acid sequence being under the regulation of a cis-acting regulatory element.

According to some embodiments of the invention, said polynucleotide acts by a mechanism selected from the group consisting of sense suppression, antisense suppresion, ribozyme inhibition, gene disruption.

According to some embodiments of the invention, said cis-acting regulatory element comprises a promoter.

According to some embodiments of the invention, said promoter comprises a tissue-specific promoter.

According to some embodiments of the invention, said tissue-specific promoter comprises a root specific promoter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme of a binary vector that can be used according to some embodiments of the invention;

FIGS. 2A-J are schematic illustrations of some of the miRNA sequences which may be used in accordance with the present invention.

### DESCRIPTION OF SPECIFIC EMBODIMENTS OF THE INVENTION

The present invention, in some embodiments thereof, relates to isolated polynucleotides expressing or modulating microRNAs or targets of same, transgenic plants comprising same and uses thereof in improving nitrogen use efficiency, abiotic stress tolerance, biomass, vigor or yield of a plant.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The doubling of agricultural food production worldwide over the past four decades has been associated with a 7-fold increase in the use of nitrogen (N) fertilizers. As a consequence, both the recent and future intensification of the use of nitrogen fertilizers in agriculture already has and will continue to have major detrimental impacts on the diversity and functioning of the non-agricultural neighbouring bacterial, animal, and plant ecosystems. The most typical examples of such an impact are the eutrophication of freshwater and marine ecosystems as a result of leaching when high rates of nitrogen fertilizers are applied to agricultural fields. In addition, there can be gaseous emission of nitrogen oxides reacting with the stratospheric ozone and the emission of toxic ammonia into the atmosphere. Furthermore, farmers are facing increasing economic pressures with the rising fossil fuels costs required for production of nitrogen fertilizers.

It is therefore of major importance to identify the critical steps controlling plant nitrogen use efficiency (NUE). Such studies can be harnessed towards generating new energy crop species that have a larger capacity to produce biomass with the minimal amount of nitrogen fertilizer.

While reducing the present invention to practice, the present inventors have uncovered microRNA (miRNA) sequences that are differentially expressed in maize plants grown under nitrogen limiting conditions versus maize plants grown under conditions wherein nitrogen is a non-limiting factor. Following extensive experimentation and screening the present inventors have identified miRNA sequences that are upregulated or downregulated in roots and leaves, and suggest using same or sequences controlling same in the generation of transgenic plants having improved nitrogen use efficiency. While further reducing the present invention to practice, the present inventors have analyzed the level of expression of the identified miRNA sequences under optima, and nitrogen deficient conditions by quantitiative RT-PCR and validated the correlation between miRNA expression nitrogen availability. These findings support the use of the miRNA sequences or sequences controlling same or targets thereof in the generation of transgenic plants characterized by improved nitrogen use efficiency and abiotic stress tolerance.

According to some embodiments, the newly uncovered miRNA sequences relay their effect by affecting at least one of:

root architecture so as to increase nutrient uptake;

activation of plant metabolic pathways so as to maximize nitrogen absorption or localization; or alternatively or additionally

modulating plant surface permeability.

Each of the above mechanisms may affect water uptake as well as salt absorption and therefore embodiments of the invention further relate to enhancement of abiotic stress tolerance, biomass, vigor or yield of the plant.

Thus, according to an aspect of the invention there is provided a method of improving nitrogen use efficiency, abiotic stress tolerance, biomass, vigor or yield of a plant, the method comprising expressing within the plant an exogenous polynucleotide having a nucleic acid sequence at least 80%, 85%, 90% or 95% identical to SEQ ID NOs: 10, 6-9 and 23-37 wherein said nucleic acid sequence is capable of regulating nitrogen use efficiency of the plant, thereby improving nitrogen use efficiency, abiotic stress tolerance, biomass, vigor or yield of the plant.

According to a specific embodiment the exogenous polynucleotide has a nucleic acid sequence at least 90% identical to SEQ ID NOs: 10, 6-9, 23-37.

According to a specific embodiment the exogenous polynucleotide has a nucleic acid sequence at least 95% identical to SEQ ID NOs: 10, 6-9, 23-37.

According to a specific embodiment the exogenous polynucleotide has a nucleic acid sequence as set forth in SEQ ID NOs: 10, 6-9, 23-37.

As used herein the phrase “nitrogen use efficiency (NUE)” refers to a measure of crop production per unit of nitrogen fertilizer input. Fertilizer use efficiency (FUE) is a measure of NUE. Crop production can be measured by biomass, vigor or yield. The plant's nitrogen use efficiency is typically a result of an alteration in at least one of the uptake, spread, absorbance, accumulation, relocation (within the plant) and use of nitrogen absorbed by the plant. Improved NUE is with respect to that of a non-transgenic plant (i.e., lacking the transgene of the transgenic plant) of the same species and of the same developmental stage and grown under the same conditions.

As used herein the phrase “nitrogen-limiting conditions” refers to growth conditions which include a level (e.g., concentration) of nitrogen (e.g., ammonium or nitrate) applied which is below the level needed for optimal plant metabolism, growth, reproduction and/or viability.

The phrase “abiotic stress” as used herein refers to any adverse effect on metabolism, growth, viability and/or reproduction of a plant. Abiotic stress can be induced by any of suboptimal environmental growth conditions such as, for example, water deficit or drought, flooding, freezing, low or high temperature, strong winds, heavy metal toxicity, anaerobiosis, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present invention contemplates situations in which there is a single abiotic stress condition or alternatively situations in which two or more abiotic stresses occur.

According to an exemplary embodiment the abiotic stress refers to salinity.

According to another exemplary embodiment the abiotic stress refers to drought.

As used herein the phrase “abiotic stress tolerance” refers to the ability of a plant to endure an abiotic stress without exhibiting substantial physiological or physical damage (e.g. alteration in metabolism, growth, viability and/or reproductivity of the plant).

As used herein the term/phrase “biomass”, “biomass of a plant” or “plant biomass” refers to the amount (e.g., measured in grams of air-dry tissue) of a tissue produced from the plant in a growing season. An increase in plant biomass can be in the whole plant or in parts thereof such as aboveground (e.g. harvestable) parts, vegetative biomass, roots and/or seeds.

As used herein the term/phrase “vigor”, “vigor of a plant” or “plant vigor” refers to the amount (e.g., measured by weight) of tissue produced by the plant in a given time. Increased vigor could determine or affect the plant yield or the yield per growing time or growing area. In addition, early vigor (e.g. seed and/or seedling) results in improved field stand.

As used herein the term/phrase “yield”, “yield of a plant” or “plant yield” refers to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

According to an exemplary embodiment the yield is measured by cellulose content.

According to another exemplary embodiment the yield is measured by oil content.

According to another exemplary embodiment the yield is measured by protein content.

According to another exemplary embodiment, the yield is measured by seed number per plant or part thereof (e.g., kernel). A plant yield can be affected by various parameters including, but not limited to, plant biomass; plant vigor; plant growth rate; seed yield; seed or grain quantity; seed or grain quality; oil yield; content of oil, starch and/or protein in harvested organs (e.g., seeds or vegetative parts of the plant); number of flowers (e.g. florets) per panicle (e.g. expressed as a ratio of number of filled seeds over number of primary panicles); harvest index; number of plants grown per area; number and size of harvested organs per plant and per area; number of plants per growing area (e.g. density); number of harvested organs in field; total leaf area; carbon assimilation and carbon partitioning (e.g. the distribution/allocation of carbon within the plant); resistance to shade; number of harvestable organs (e.g. seeds), seeds per pod, weight per seed; and modified architecture [such as increase stalk diameter, thickness or improvement of physical properties (e.g. elasticity)].

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in NUE, in tolerance to abiotic stress, in yield, in biomass or in vigor of a plant, as compared to a native or wild-type plants [i.e., plants not genetically modified to express the biomolecules (polynucleotides) of the invention, e.g., a non-transformed plant of the same species and of the same developmental stage which is grown under the same growth conditions as the transformed plant].

Improved plant NUE is translated in the field into either harvesting similar quantities of yield, while implementing less fertilizers, or increased yields gained by implementing the same levels of fertilizers. Thus, improved NUE or FUE has a direct effect on plant yield in the field.

The term “plant” as used herein encompasses whole plants, ancestors and progeny of the plants and plant parts, including seeds, shoots, stems, roots (including tubers), and isolated plant cells, tissues and organs. The plant may be in any form including suspension cultures, embryos, meristematic regions, callus tissue, leaves, gametophytes, sporophytes, pollen, and microspores.

As used herein the phrase “plant cell” refers to plant cells which are derived and isolated from disintegrated plant cell tissue or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

Any commercially or scientifically valuable plant is envisaged in accordance with these embodiments of the invention. Plants that are particularly useful in the methods of the invention include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barely, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively algae and other non-Viridiplantae can be used for the methods of the present invention.

According to some embodiments of the invention, the plant used by the method of the invention is a crop plant including, but not limited to, cotton, Brassica vegetables, oilseed rape, sesame, olive tree, palm oil, banana, wheat, corn or maize, barley, alfalfa, peanuts, sunflowers, rice, oats, sugarcane, soybean, turf grasses, barley, rye, sorghum, sugar cane, chicory, lettuce, tomato, zucchini, bell pepper, eggplant, cucumber, melon, watermelon, beans, hibiscus, okra, apple, rose, strawberry, chile, garlic, pea, lentil, canola, mums, arabidopsis, broccoli, cabbage, beet, quinoa, spinach, squash, onion, leek, tobacco, potato, sugarbeet, papaya, pineapple, mango, Arabidopsis thaliana, and also plants used in horticulture, floriculture or forestry, such as, but not limited to, poplar, fir, eucalyptus, pine, an ornamental plant, a perennial grass and a forage crop, coniferous plants, moss, algae, as well as other plants listed in World Wide Web (dot) nationmaster (dot) com/encyclopedia/Plantae.

According to a specific embodiment of the present invention, the plant comprises corn.

According to a specific embodiment of the present invention, the plant comprises sorghum.

As used herein, the phrase “exogenous polynucleotide” refers to a heterologous nucleic acid sequence which may not be naturally expressed within the plant or which overexpression in the plant is desired. The exogenous polynucleotide may be introduced into the plant in a stable or transient manner, so as to produce a ribonucleic acid (RNA) molecule. It should be noted that the exogenous polynucleotide may comprise a nucleic acid sequence which is identical or partially homologous to an endogenous nucleic acid sequence of the plant.

As mentioned the present teachings are based on the identification of miRNA sequences which modulate nitrogen use efficiency of plants.

According to some embodiments the exogenous polynucleotide encodes a miRNA or a precursor thereof.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds. Exemplary hairpin sequences are provided in Tables 1, 3 and 4, below.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the miRNA molecules may be naturally occurring or synthetic.

Thus, the present teachings contemplate expressing an exogenous polynucleotide having a nucleic acid sequence at least 90%, 91%, 92%, 93%, 94%, 95%, 96%, 97%, 98% 99% or 100% identical to SEQ ID NOs 1-10, 23-37, 57-449, provided that they regulate nitrogen use efficiency.

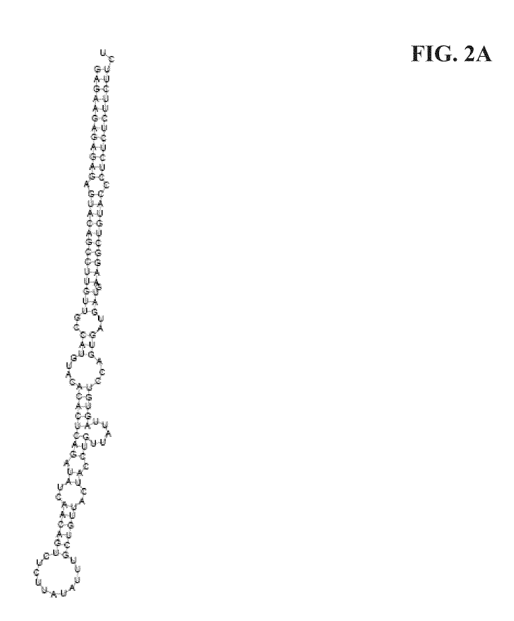

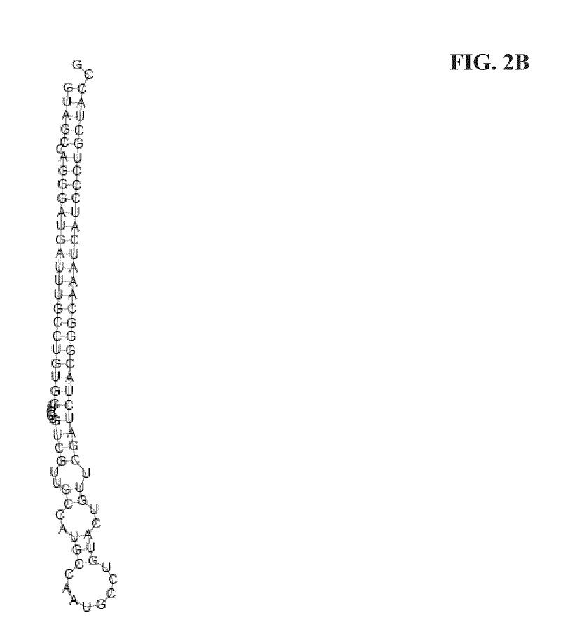

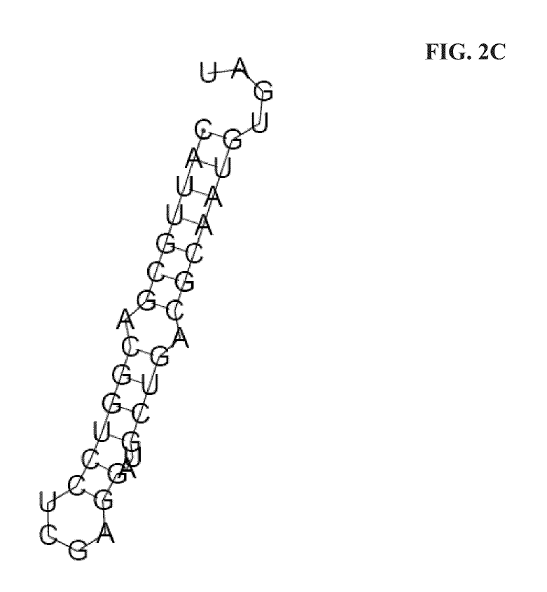

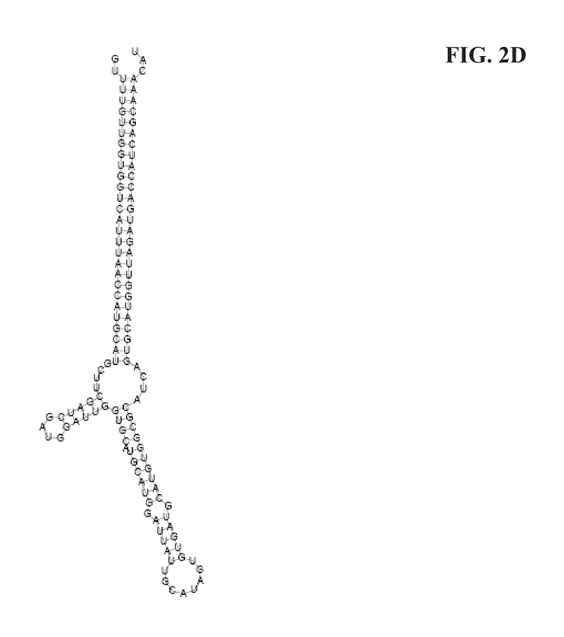

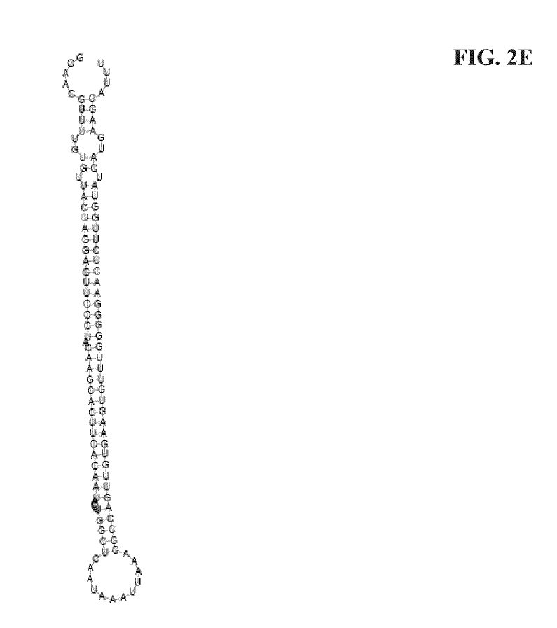

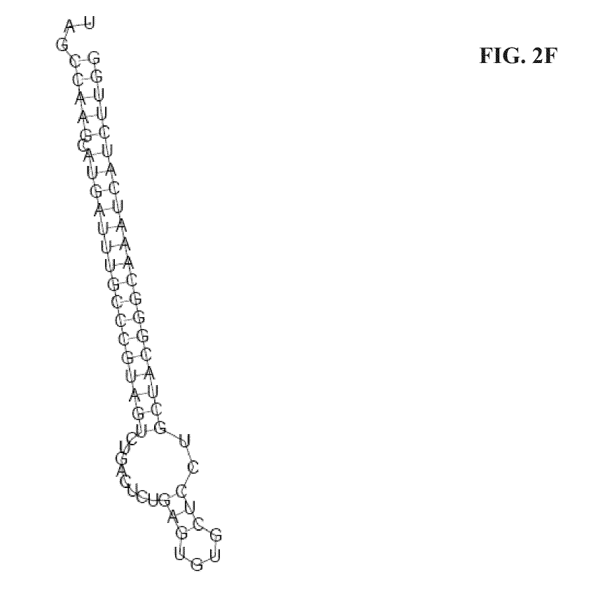

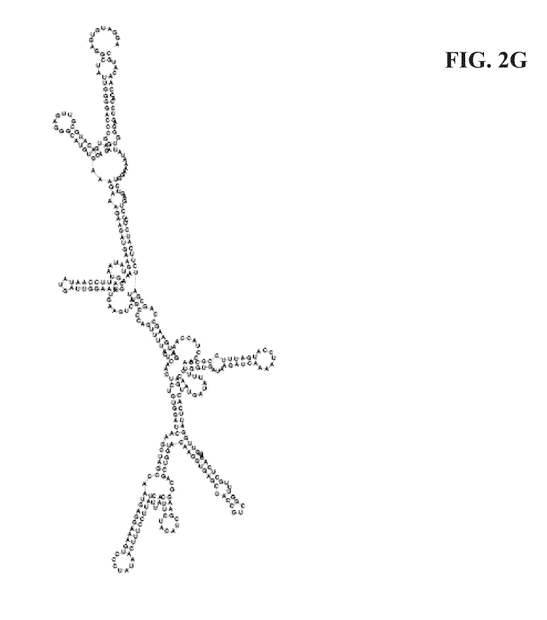

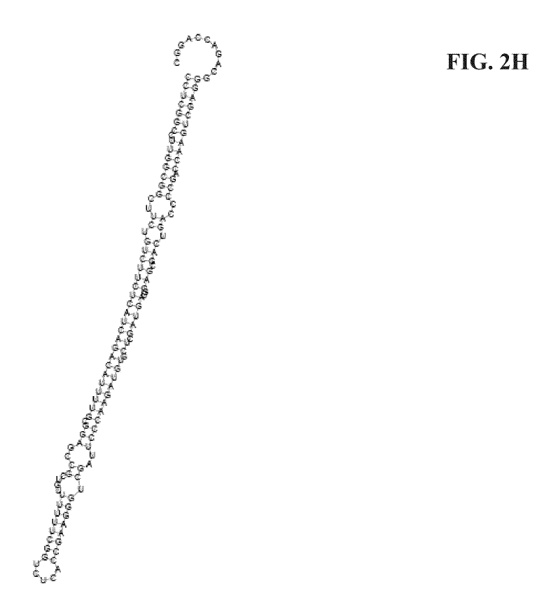

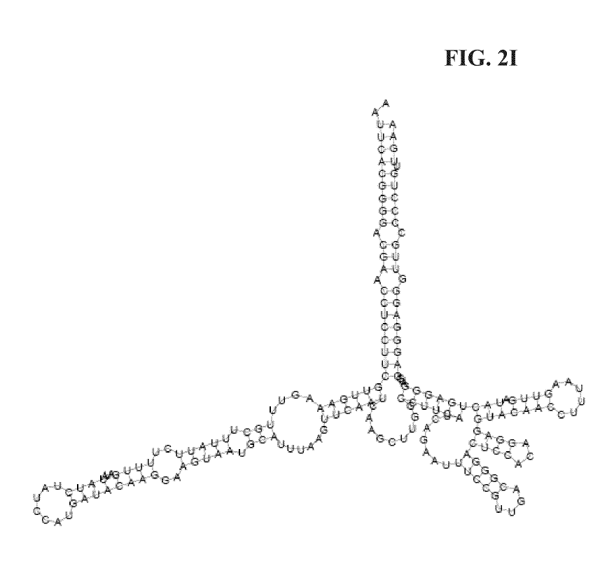

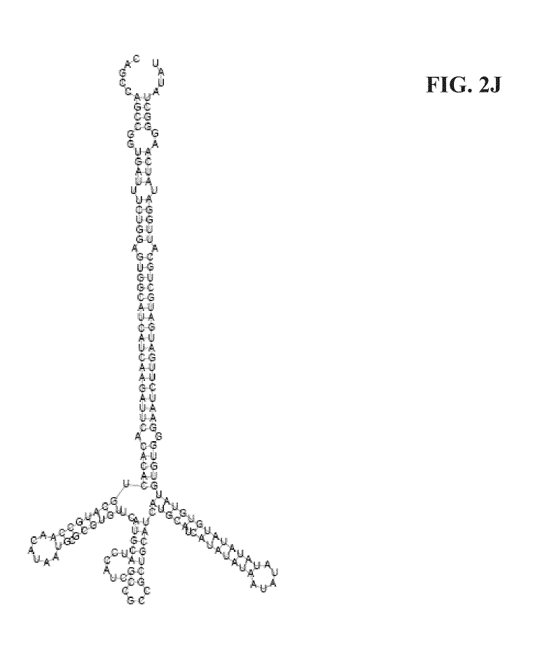

Alternatively or additionally, the present teachings contemplate expressing an exogenous polynucleotide having a nucleic acid sequence at least 65%, 50%, 75%, 80%, 85%, 90%, 91%, 92%, 93%, 94%, 95%, 96%, 97%, 98% 99% or 100% identical to SEQ ID NOs. 1-10, 21 and 22 (mature and precursors Tables 1 and 3, and FIGS. 2A-H representing the core maize genes), provided that they regulate nitrogen use efficiency.

Tables 1 and 3 below illustrates exemplary miRNA sequences and precursors thereof which over expression are associated with modulation of nitrogen use efficiency.

The present invention envisages the use of homologous and orthologous sequences of the above miRNA molecules. At the precursor level use of homologous sequences can be done to a much broader extend. Thus, in such precursor sequences the degree of homology may be lower in all those sequences not including the mature miRNA segment therein.

As used herein, the phrase “stem-loop precursor” refers to stem loop precursor RNA structure from which the miRNA can be processed.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Thus, according to a specific embodiment, the exogenous polynucleotide encodes a stem-loop precursor of the nucleic acid sequence. Such a stem-loop precursor can be at least about 60%, at least about 65%, at least about 70%, at least about 75%, at least about 80%, at least about 85%, at least about 90%, at least about 95% or more identical to SEQ ID NOs: 21-22, 38-52, 1209, 1211, 1212, 454-846, 53-56, 1209 (homologs precursor Tables 1 and 3 and FIGS. 2A-H), provided that it regulates nitrogen use efficiency.

Identity (e.g., percent identity) can be determined using any homology comparison software, including for example, the BlastN software of the National Center of Biotechnology Information (NCBI) such as by using default parameters.

Homology (e.g., percent homology, identity+similarity) can be determined using any homology comparison software, including for example, the TBLASTN software of the National Center of Biotechnology Information (NCBI) such as by using default parameters.

According to some embodiments of the invention, the term “homology” or “homologous” refers to identity of two or more nucleic acid sequences; or identity of two or more amino acid sequences.

Homologous sequences include both orthologous and paralogous sequences. The term “paralogous” relates to gene-duplications within the genome of a species leading to paralogous genes. The term “orthologous” relates to homologous genes in different organisms due to ancestral relationship.

One option to identify orthologues in monocot plant species is by performing a reciprocal blast search. This may be done by a first blast involving blasting the sequence-of-interest against any sequence database, such as the publicly available NCBI database which may be found at: Hypertext Transfer Protocol://World Wide Web (dot) ncbi (dot) nlm (dot) nih (dot) gov. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [Hypertext Transfer Protocol://World Wide Web (dot) ebi (dot) ac (dot) uk/Tools/clustalw2/index (dot) html], followed by a neighbor-joining tree (Hypertext Transfer Protocol://en (dot) wikipedia (dot) org/wiki/Neighbor-joining) which helps visualizing the clustering.

Interestingly, while screening for RNAi regulatory sequences, the present inventors have identified a number of miRNA sequences which have never been described before.

Thus, according to an aspect of the invention there is provided an isolated polynucleotide having a nucleic acid sequence at least 80%, 85% or preferably 90%, 91%, 92%, 93%, 94%, 95%, 96%, 97%, 98% 99% or 100% identical to SEQ ID NO: 6, 7, 9, 1209, 1210, 1211, 1212 (Table 1 predicted both upregulated and downregulated), wherein said nucleic acid sequence is capable of regulating nitrogen use efficiency of a plant.

According to a specific embodiment, the isolated polynucleotide encodes a stem-loop precursor of the nucleic acid sequence.

According to a specific embodiment, the stem-loop precursor is at least about 60%, at least about 65%, at least about 70%, at least about 75%, at least about 80%, at least about 85%, at least about 90%, at least about 95% or more identical to the precursor sequence of SEQ ID NOs: 21, 22, 38-52, 1209, 1211, 1212, 454-846 and 53-56, 1209 (predicted stem and loop), provided that it regulates nitrogen use efficiency.

As mentioned, the present inventors have also identified RNAi sequences which are down regulated under nitrogen limiting conditions.

Thus, according to an aspect of the invention there is provided a method of improving nitrogen use efficiency, abiotic stress tolerance, biomass, vigor or yield of a plant, the method comprising expressing within the plant an exogenous polynucleotide which downregulates an activity or expression of a gene encoding a miRNA molecule having a nucleic acid sequence at least 80%, 85% or preferably 90%, 95% or even 100% identical to the sequence selected from the group consisting of SEQ ID NOs: 4, 1-3,5,53-56, 1209, 57-449, 454-846 (Tables 1 and 4 down-regulated), thereby improving nitrogen use efficiency, abiotic stress tolerance, biomass, vigor or yield of a plant

There are various approaches to down regulate miRNA sequences.

As used herein the term “down-regulation” refers to reduced activity or expression of the miRNA (at least 10%, 20%, 30%, 50%, 60%, 70%, 80%, 90% or 100% reduction in activity or expression) as compared to its activity or expression in a plant of the same species and the same developmental stage not expressing the exogenous polynucleotide.

Nucleic acid agents that down-regulate miR activity include, but are not limited to, a target mimic, a micro-RNA resistant gene and a miRNA inhibitor.

The target mimic or micro-RNA resistant target is essentially complementary to the microRNA provided that one or more of following mismatches are allowed:

(a) a mismatch between the nucleotide at the 5′ end of the microRNA and the corresponding nucleotide sequence in the target mimic or micro-RNA resistant target;

(b) a mismatch between any one of the nucleotides in position 1 to position 9 of the microRNA and the corresponding nucleotide sequence in the target mimic or micro-RNA resistant target; or

(c) three mismatches between any one of the nucleotides in position 12 to position 21 of the microRNA and the corresponding nucleotide sequence in the target mimic or micro-RNA resistant target provided that there are no more than two consecutive mismatches.

The target mimic RNA is essentially similar to the target RNA modified to render it resistant to miRNA induced cleavage, e.g. by modifying the sequence thereof such that a variation is introduced in the nucleotide of the target sequence complementary to the nucleotides 10 or 11 of the miRNA resulting in a mismatch.

Alternatively, a microRNA-resistant target may be implemented. Thus, a silent mutation may be introduced in the microRNA binding site of the target gene so that the DNA and resulting RNA sequences are changed in a way that prevents microRNA binding, but the amino acid sequence of the protein is unchanged. Thus, a new sequence can be synthesized instead of the existing binding site, in which the DNA sequence is changed, resulting in lack of miRNA binding to its target.

Tables 10 and 11 below provide non-limiting examples of target mimics and target resistant sequences that can be used to down-regulate the activity of the miRs of the invention.

According to a specific embodiment, the target mimic or micro-RNA resistant target is linked to the promoter naturally associated with the pre-miRNA recognizing the target gene and introduced into the plant cell. In this way, the miRNA target mimic or micro-RNA resistant target RNA will be expressed under the same circumstances as the miRNA and the target mimic or micro-RNA resistant target RNA will substitute for the non-target mimic/micro-RNA resistant target RNA degraded by the miRNA induced cleavage.

Non-functional miRNA alleles or miRNA resistant target genes may also be introduced by homologous recombination to substitute the miRNA encoding alleles or miRNA sensitive target genes.

Recombinant expression is effected by cloning the nucleic acid of interest (e.g., miRNA, target gene, silencing agent etc) into a nucleic acid expression construct under the expression of a plant promoter, as further described hereinbelow.

In other embodiments of the invention, synthetic single stranded nucleic acids are used as miRNA inhibitors. A miRNA inhibitor is typically between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA.

While further reducing the present invention to practice, the present inventors have identified gene targets for the differentially expressed miRNA molecules. It is therefore contemplated, that gene targets of those miRNAs that are down regulated during stress should be overexpressed in order to confer tolerance, while gene targets of those miRNAs that are up regulated during stress should be downregulated in the plant in order to confer tolerance.

Thus, according to an aspect of the invention there is provided a method of improving nitrogen use efficiency, abiotic stress tolerance, biomass, vigor or yield of a plant, the method comprising expressing within the plant an exogenous polynucleotide encoding a polypeptide having an amino acid sequence at least 80%, 82%, 84%, 85%, 86%, 88%, 90%, 92%, 94%, 95%, 96%, 97%, 98%, 99% or 100% homologous to SEQ ID NOs: 927-1021 (gene targets of down regulated miRNAs, see Table 6), wherein said polypeptide is capable of regulating nitrogen use efficiency of the plant, thereby improving nitrogen use efficiency, abiotic stress tolerance, biomass, vigor or yield of the plant.

Nucleic acid sequences (also referred to herein as polynucleotides) of the polypeptides of some embodiments of the invention may be optimized for expression in a specific plant host. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn—Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www.kazusa.or.jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Target genes which are contemplated according to the present teachings are provided in the polynucleotide sequences which comprise nucleic acid sequences as set forth in the maize polynucleotides listed in Tables 5 and 6). However the present teachings also relate to orthologs or homologs at least about 60%, at least about 65%, at least about 70%, at least about 75%, at least about 80%, at least about 85%, at least about 90%, or at least about 95% or more identical or similar to SEQ ID NO: 895-926 or 1022-1090 (polynucleotides listed in Tables 5 and 6). Parameters for determining the level of identity are provided hereinbelow.

Alternatively or additionally, target genes which are contemplated according to the present teachings are provided in the polypeptide sequences which comprise amino acid sequences as set forth the maize polypeptides of Tables 5 and 6). However the present teachings also relate to of orthologs or homologs at least about 60%, at least about 65%, at least about 70%, at least about 75%, at least about 80%, at least about 85%, at least about 90%, or at least about 95% or more identical or similar to SEQ ID NO: 854-894 or 927-1021 (Tables 5 and 6).

Homology (e.g., percent homology, identity+similarity) can be determined using any homology comparison software, including for example, the TBLASTN software of the National Center of Biotechnology Information (NCBI) such as by using default parameters, when starting from a polypeptide sequence; or the tBLASTX algorithm (available via the NCBI) such as by using default parameters, which compares the six-frame conceptual translation products of a nucleotide query sequence (both strands) against a protein sequence database.

According to some embodiments of the invention, the term “homology” or “homologous” refers to identity of two or more nucleic acid sequences; or identity of two or more amino acid sequences.

Homologous sequences include both orthologous and paralogous sequences. The term “paralogous” relates to gene-duplications within the genome of a species leading to paralogous genes. The term “orthologous” relates to homologous genes in different organisms due to ancestral relationship.

One option to identify orthologues in monocot plant species is by performing a reciprocal blast search. This may be done by a first blast involving blasting the sequence-of-interest against any sequence database, such as the publicly available NCBI database which may be found at: Hypertext Transfer Protocol://World Wide Web (dot) ncbi (dot) nlm (dot) nih (dot) gov. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [Hypertext Transfer Protocol://World Wide Web (dot) ebi (dot) ac (dot) uk/Tools/clustalw2/index (dot) html], followed by a neighbor-joining tree (Hypertext Transfer Protocol://en (dot) wikipedia (dot) org/wiki/Neighbor-joining) which helps visualizing the clustering.

As mentioned the present inventors have also identified genes which down-regulation may be done in order to improve their NUE, biomass, vigor, yield and abiotic stress tolerance.

Thus, according to an aspect of the invention there is provided a method of improving nitrogen use efficiency, abiotic stress tolerance, biomass, vigor or yield of a plant, the method comprising expressing within the plant an exogenous polynucleotide which downregulates an activity or expression of a polypeptide having an amino acid sequence at least 80%, 85%, 90%, 95%, or 100% homologous to SEQ ID NOs: 854-894 (polypeptides of Table 5), wherein said polypeptide is capable of regulating nitrogen use efficiency of the plant, thereby improving nitrogen use efficiency, abiotic stress tolerance, biomass, vigor or yield of the plant.

Down regulation of activity or expression is by at least 10%, 20%, 30%, 40%, 50%, 60%, 70%, 80%, 90% or even complete (100%) loss of activity or expression. Assays for measuring gene expression can be effected at the protein level (e.g., Western blot, ELISA) or at the mRNA level such as by RT-PCR.

According to a specific embodiment the amino acid sequence of the target gene is as set forth in SEQ ID NOs: 854-894 of Table 5.

Alternatively or additionally, the amino acid sequence of the target gene is encoded by a polynucleotide sequence as set forth in SEQ ID NOs: 895-926 of Table 5.

Examples of polynucleotide downregulating agents that inhibit (also referred to herein as inhibitors or nucleic acid agents) the expression of a target gene are given below.

1. Polynucleotide-Based Inhibition of Gene Expression.

It will be appreciated, that any of these methods when specifically referring to downregulating expression/activity of the target genes can be used, at least in part, to downregulate expression or activity of endogenous RNA molecules.

i. Sense Suppression/Cosuppression

In some embodiments of the invention, inhibition of the expression of target gene may be obtained by sense suppression or cosuppression. For cosuppression, an expression cassette is designed to express an RNA molecule corresponding to all or part of a messenger RNA encoding a target gene in the “sense” orientation. Over-expression of the RNA molecule can result in reduced expression of the native gene. Accordingly, multiple plant lines transformed with the cosuppression expression cassette are screened to identify those that show the greatest inhibition of target gene expression.

The polynucleotide used for cosuppression may correspond to all or part of the sequence encoding the target gene, all or part of the 5′ and/or 3′ untranslated region of a target transcript, or all or part of both the coding sequence and the untranslated regions of a transcript encoding the target gene. In some embodiments where the polynucleotide comprises all or part of the coding region for the target gene, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin, et al., (2002) Plant Cell 15:1517-1532. Cosuppression may also be used to inhibit the expression of multiple proteins in the same plant. Methods for using cosuppression to inhibit the expression of endogenous genes in plants are described in Flavell, et al., (1995) Proc. Natl. Acad. Sci. USA 91:3590-3596; Jorgensen, et al., (1996) Plant Mol. Biol. 31:957-973; Johansen and Carrington, (2001) Plant Physiol. 126:930-938; Broin, et al., (2002) Plant Cell 15:1517-1532; Stoutjesdijk, et al., (2002) Plant Physiol. 129:1723-1731; Yu, et al., (2003) Phytochemistry 63:753-763; and U.S. Pat. Nos. 5,035,323, 5,283,185 and 5,952,657; each of which is herein incorporated by reference. The efficiency of cosuppression may be increased by including a poly-dt region in the expression cassette at a position 3′ to the sense sequence and 5′ of the polyadenylation signal. See, US Patent Publication Number 20020058815, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,185 and 5,035,323; herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing. (Aufsatz, et al., (2002) PNAS 99(4):16499-16506; Mette, et al., (2000) EMBO J. 19(19):5194-5201)

ii. Antisense Suppression

In some embodiments of the invention, inhibition of the expression of the target gene may be obtained by antisense suppression. For antisense suppression, the expression cassette is designed to express an RNA molecule complementary to all or part of a messenger RNA encoding the target gene. Over-expression of the antisense RNA molecule can result in reduced expression of the native gene. Accordingly, multiple plant lines transformed with the antisense suppression expression cassette are screened to identify those that show the greatest inhibition of target gene expression.

The polynucleotide for use in antisense suppression may correspond to all or part of the complement of the sequence encoding the target gene, all or part of the complement of the 5′ and/or 3′ untranslated region of the target gene transcript, or all or part of the complement of both the coding sequence and the untranslated regions of a transcript encoding the target gene. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. Furthermore, portions of the antisense nucleotides may be used to disrupt the expression of the target gene. Generally, sequences of at least 50 nucleotides, 100 nucleotides, 200 nucleotides, 300, 500, 550, 500, 550 or greater may be used. Methods for using antisense suppression to inhibit the expression of endogenous genes in plants are described, for example, in Liu, et al., (2002) Plant Physiol. 129:1732-1753 and U.S. Pat. No. 5,759,829, which is herein incorporated by reference. Efficiency of antisense suppression may be increased by including a poly-dt region in the expression cassette at a position 3′ to the antisense sequence and 5′ of the polyadenylation signal. See, US Patent Publication Number 20020058815.

iii. Double-Stranded RNA Interference

In some embodiments of the invention, inhibition of the expression of a target gene may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of target gene expression. Methods for using dsRNA interference to inhibit the expression of endogenous plant genes are described in Waterhouse, et al., (1998) Proc. Natl. Acad. Sci. USA 95:13959-13965, Liu, et al., (2002) Plant Physiol. 129:1732-1753, and WO 99/59029, WO 99/53050, WO 99/61631, and WO 00/59035;

iv. Hairpin RNA Interference and Intron-Containing Hairpin RNA Interference

In some embodiments of the invention, inhibition of the expression of one or more target gene may be obtained by hairpin RNA (hpRNA) interference or intron-containing hairpin RNA (ihpRNA) interference. These methods are highly efficient at downregulating the expression of endogenous genes. See, Waterhouse and Helliwell, (2003) Nat. Rev. Genet. 5:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:5985-5990; Stoutjesdijk, et al., (2002) Plant Physiol. 129:1723-1731; and Waterhouse and Helliwell, (2003) Nat. Rev. Genet. 5:29-38. Methods for using hpRNA interference to inhibit or silence the expression of genes are described, for example, in Chuang and Meyerowitz, (2000) Proc. Natl. Acad. Sci. USA 97:5985-5990; Stoutjesdijk, et al., (2002) Plant Physiol. 129:1723-1731; Waterhouse and Helliwell, (2003) Nat. Rev. Genet. 5:29-38; Pandolfini, et al., BMC Biotechnology 3:7, and US Patent Publication Number 20030175965; each of which is herein incorporated by reference. A transient assay for the efficiency of hpRNA constructs to silence gene expression in vivo has been described by Panstruga, et al., (2003) Mol. Biol. Rep. 30:135-150, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 507:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 507:319-320; Wesley, et al., (2001) Plant J. 27:584, 1-3, 590; Wang and Waterhouse, (2001) Curr. Opin. Plant Biol. 5:156-150; Waterhouse and Helliwell, (2003) Nat. Rev. Genet. 5:29-38; Helliwell and Waterhouse, (2003) Methods 30:289-295, and US Patent Publication Number 20030180955, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00905, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for target gene). Methods of using amplicons to inhibit the expression of endogenous plant genes are described, for example, in Angell and Baulcombe, (1997) EMBO J. 16:3675-3685, Angell and Baulcombe, (1999) Plant J. 20:357-362, and U.S. Pat. No. 6,656,805, each of which is herein incorporated by reference.

vi. Ribozymes

In some embodiments, the polynucleotide expressed by the expression cassette of the invention is catalytic RNA or has ribozyme activity specific for the messenger RNA of target gene. Thus, the polynucleotide causes the degradation of the endogenous messenger RNA, resulting in reduced expression of the target gene. This method is described, for example, in U.S. Pat. No. 5,987,071, herein incorporated by reference.

2. Gene Disruption

In some embodiments of the present invention, the activity of a miRNA or a target gene is reduced or eliminated by disrupting the gene encoding the target polypeptide. The gene encoding the target polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have reduced response regulator activity.

Any of the nucleic acid agents described herein (for overexpression or downregulation of either the target gene of the miRNA) can be provided to the plant as naked RNA or expressed from a nucleic acid expression construct, where it is operaly linked to a regulatory sequence.

According to a specific embodiment of the invention, there is provided a nucleic acid construct comprising a nucleic acid sequence encoding a the nucleic acid agent (e.g., miRNA or a precursor thereof as described herein, gene targetm or silencing agent), said nucleic acid sequence being under a transcriptional control of a regulatory sequence such as a tissue specific promoter.

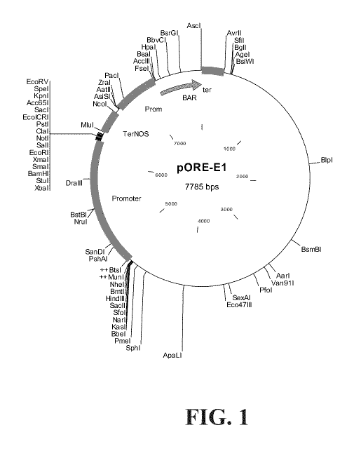

An exemplary nucleic acid construct which can be used for plant transformation include, the pORE E2 binary vector (FIG. 1) in which the relevant nucleic acid sequence is ligated under the transcriptional control of a promoter.

A coding nucleic acid sequence is “operably linked” or “transcriptionally linked to a regulatory sequence (e.g., promoter)” if the regulatory sequence is capable of exerting a regulatory effect on the coding sequence linked thereto. Thus, the regulatory sequence controls the transcription of the miRNA or precursor thereof, gene target or silencing agent.

The term “regulatory sequence”, as used herein, means any DNA, that is involved in driving transcription and controlling (i.e., regulating) the timing and level of transcription of a given DNA sequence, such as a DNA coding for a miRNA, precursor or inhibitor of same. For example, a 5′ regulatory region (or “promoter region”) is a DNA sequence located upstream (i.e., 5′) of a coding sequence and which comprises the promoter and the 5′-untranslated leader sequence. A 3′ regulatory region is a DNA sequence located downstream (i.e., 3′) of the coding sequence and which comprises suitable transcription termination (and/or regulation) signals, including one or more polyadenylation signals.

For the purpose of the invention, the promoter is a plant-expressible promoter. As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin Thus, any suitable promoter sequence can be used by the nucleic acid construct of the present invention. According to some embodiments of the invention, the promoter is a constitutive promoter, a tissue-specific promoter or an inducible promoter (e.g. an abiotic stress-inducible promoter).

Suitable constitutive promoters include, for example, hydroperoxide lyase (HPL) promoter, CaMV 35S promoter (Odell et al, Nature 313:810-812, 1985); Arabidopsis At6669 promoter (see PCT Publication No. WO04081173A2); Arabidopsis new At6669 promoter; maize Ubi 1 (Christensen et al., Plant Sol. Biol. 18:675-689, 1992); rice actin (McElroy et al., Plant Cell 2:163-171, 1990); pEMU (Last et al, Theor. Appl. Genet. 81:584, 1-3, 588, 1991); CaMV 19S (Nilsson et al, Physiol. Plant 100:456-462, 1997); GOS2 (de Pater et al, Plant J November; 2(6):837-44, 1992); ubiquitin (Christensen et al, Plant MoI. Biol. 18: 675-689, 1992); Rice cyclophilin (Bucholz et al, Plant MoI Biol. 25(5):837-43, 1994); Maize H3 histone (Lepetit et al, MoI. Gen. Genet. 231: 276-285, 1992); Actin 2 (An et al, Plant J. 10(1); 107-121, 1996) and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable tissue-specific promoters include, but not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant MoI. Biol. 23:1129-1138, 1993; and Matsuoka et al., Proc. Natl. Acad. Sci. USA 90:9586-9590, 1993], seed-preferred promoters [e.g., from seed specific genes (Simon, et al., Plant MoI. Biol. 5. 191, 1985; Scofield, et al., J. Biol. Chem. 262: 12202, 1987; Baszczynski, et al., Plant MoI. Biol. 14: 633, 1990), Brazil Nut albumin (Pearson' et al., Plant MoI. Biol. 18: 235-245, 1992), legumin (Ellis, et al. Plant MoI. Biol. 10: 203-214, 1988), Glutelin (rice) (Takaiwa, et al., MoI. Gen. Genet. 208: 15-22, 1986; Takaiwa, et al., FEBS Letts. 221: 43-47, 1987), Zein (Matzke et al., Plant MoI Biol, 143)323-32 1990), napA (Stalberg, et al., Planta 199: 515-519, 1996), Wheat SPA (Albanietal, Plant Cell, 9: 171-184, 1997), sunflower oleosin (Cummins, et al, Plant MoI. Biol. 19: 873-876, 1992)], endosperm specific promoters [e.g., wheat LMW and HMW, glutenin-1 (MoI Gen Genet 216:81-90, 1989; NAR 17:461-2), wheat a, b and g gliadins (EMBO3: 1409-15, 1984), Barley ltrl promoter, barley Bl, C, D hordein (Theor Appl Gen 98:1253-62, 1999; Plant J 4:343-55, 1993; MoI Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin GIb-I (Wu et al., Plant Cell Physiology 39(8) 885-889, 1998), rice alpha-globulin REB/OHP-1 (Nakase et al. Plant MoI. Biol. 33: 513-S22, 1997), rice ADP-glucose PP (Trans Res 6:157-68, 1997), maize ESR gene family (Plant J 12:235-46, 1997), sorghum gamma-kafirin (PMB 32:1029-35, 1996); e.g., the Napin promoter], embryo specific promoters [e.g., rice OSH1 (Sato et al, Proc. Natl. Acad. Sci. USA, 93: 8117-8122), KNOX (Postma-Haarsma et al, Plant MoI. Biol. 39:257-71, 1999), rice oleosin (Wu et at, J. Biochem., 123:386, 1998)], and flower-specific promoters [e.g., AtPRP4, chalene synthase (chsA) (Van der Meer, et al., Plant MoI. Biol. 15, 95-109, 1990), LAT52 (Twell et al., MoI. Gen Genet. 217:240-245; 1989), apetala-3]. Also contemplated are root-specific promoters such as the ROOTP promoter described in Vissenberg K, et al. Plant Cell Physiol. 2005 January; 46(1):192-200.

The nucleic acid construct of some embodiments of the invention can further include an appropriate selectable marker and/or an origin of replication.

The nucleic acid construct of some embodiments of the invention can be utilized to stably or transiently transform plant cells. In stable transformation, the exogenous polynucleotide is integrated into the plant genome and as such it represents a stable and inherited trait. In transient transformation, the exogenous polynucleotide is expressed by the cell transformed but it is not integrated into the genome and as such it represents a transient trait.

When naked RNA or DNA is introduced into a cell, the polynucleotides may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

There are various methods of introducing foreign genes into both monocotyledonous and dicotyledonous plants (Potrykus, L, Annu. Rev. Plant. Physiol, Plant. MoI. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer (e.g., T-DNA using Agrobacterium tumefaciens or Agrobacterium rhizogenes); see for example, Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. See, e.g., Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to a specific embodiment of the present invention, the exogenous polynucleotide is introduced into the plant by infecting the plant with a bacteria, such as using a floral dip transformation method (as described in further detail in Example 5, of the Examples section which follows).

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. For this reason it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, Tobacco mosaic virus (TMV), brome mosaic virus (BMV) and Bean Common Mosaic Virus (BV or BCMV). Transformation of plants using plant viruses is described in U.S. Pat. No. 4,855,237 (bean golden mosaic virus; BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants are described in WO 87/06261. According to some embodiments of the invention, the virus used for transient transformations is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Galon et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols: From Virus Isolation to Transgenic Resistance (Methods in Molecular Biology (Humana Pr), VoI 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Construction of plant RNA viruses for the introduction and expression of non-viral exogenous polynucleotide sequences in plants is demonstrated by the above references as well as by Dawson, W. O. et al, Virology (1989) 172:285-292; Takamatsu et al. EMBO J. (1987) 6:307-311; French et al. Science (1986) 231:1294-1297; Takamatsu et al. FEBS Letters (1990) 269:73-76; and U.S. Pat. No. 5,316,931.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat proteins which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired sequence.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference.

Regardless of the method of transformation, propagation or regeneration, the present invention also contemplates a transgenic plant exogenously expressing the polynucleotide/nucleic acid agent of the invention.

According to a specific embodiment, the transgenic plant exogenously expresses a polynucleotide having a nucleic acid sequence at least, 80%, 85%, 90%, 95% or even 100% identical to SEQ ID NOs: 2-20, 23-37, 57-449, 21-22, 38-52, 1209, 1211, 1212, 454-846 and 53-56, 1209 (Tables 1, 3 and 4), wherein said nucleic acid sequence is capable of regulating nitrogen use efficiency of the plant.

According to further embodiments, the exogenous polynucleotide encodes a precursor of said nucleic acid sequence.

According to yet further embodiments, the stem-loop precursor is at least 60%, 65%, 70%, 75%, 80%, 85%, 90%, 95% or even 100% identical to SEQ ID NOs: 21-22, 38-52, 1209, 1211, 1212, 454-846, 53-56, 1209 (Tables 1, 3 and 4) identical to SEQ ID NO: 21-22, 38-52, 1209, 1211, 1212, 54-846 and 53-56, 1209 (precursor sequences of Tables 1, 3 and 4). More specifically the exogenous polynucleotide is selected from the group consisting of SEQ ID NO: 21-22 and 38-52, 1209, 1211, 1212 (precursor and mature sequences of upregulated Tables 1 and 3).

Alternatively, there is provided a transgenic plant exogenously expressing a polynucleotide which downregulates an activity or expression of a gene encoding a miRNA molecule having a nucleic acid sequence selected from the group consisting of SEQ ID NOs: 4, 1-3,5,57-449, 454-846 and 53-56, 1209 (downregulated Tables 1 and 4) or homologs thereof which are at least at least 80%, 85%, 90% or 95% identical to SEQ ID NOs: 4, 1-3,5,57-449, 454-846 and 53-56, 1209 (downregulated Tables 1 and 4).

More specifically, the transgenic plant expresses the nucleic acid agent of Tables 8-11.

More specifically, the transgenic plant expresses the nucleic acid agent of Tables 8 and 11.

Alternatively or additionally there is provided a transgenic plant exogenously expressing a polynucleotide encoding a polypeptide having an amino acid sequence at least 80%, 85%, 90%, 95% or even 100% homologous to SEQ ID NOs: 854-894 (polypeptides of Table 5), wherein said polypeptide is capable of regulating nitrogen use efficiency of the plant.

Alternatively or additionally there is provided a transgenic plant exogenously expressing a polynucleotide encoding a polypeptide having an amino acid sequence at least 80%, 85%, 90%, 95% or even 100% homologous to SEQ ID NOs: 927-1021 (polypeptides of Table 6), wherein said polypeptide is capable of regulating nitrogen use efficiency of the plant.

Alternatively or additionally there is provided a transgenic plant exogenously expressing a polynucleotide which downregulates an activity or expression of a polypeptide having an amino acid sequence at least 80%, 85%, 90%, 95% or even 100% homologous to SEQ ID NOs: 854-894, 927-1021 (targets of Tables 5 and 6), wherein said polypeptide is capable of regulating nitrogen use efficiency of the plant.

Also contemplated are hybrids of the above described transgenic plants. A “hybrid plant” refers to a plant or a part thereof resulting from a cross between two parent plants, wherein one parent is a genetically engineered plant of the invention (transgenic plant expressing an exogenous miRNA sequence or a precursor thereof). Such a cross can occur naturally by, for example, sexual reproduction, or artificially by, for example, in vitro nuclear fusion. Methods of plant breeding are well-known and within the level of one of ordinary skill in the art of plant biology.

Since nitrogen use efficiency, abiotic stress tolerance as well as yield, vigor or biomass of the plant can involve multiple genes acting additively or in synergy (see, for example, in Quesda et al., Plant Physiol. 130:951-063, 2002), the invention also envisages expressing a plurality of exogenous polynucleotides in a single host plant to thereby achieve superior effect on the efficiency of nitrogen use, yield, vigor and biomass of the plant.

Expressing a plurality of exogenous polynucleotides in a single host plant can be effected by co-introducing multiple nucleic acid constructs, each including a different exogenous polynucleotide, into a single plant cell. The transformed cell can then be regenerated into a mature plant using the methods described hereinabove. Alternatively, expressing a plurality of exogenous polynucleotides in a single host plant can be effected by co-introducing into a single plant-cell a single nucleic-acid construct including a plurality of different exogenous polynucleotides. Such a construct can be designed with a single promoter sequence which can transcribe a polycistronic messenger RNA including all the different exogenous polynucleotide sequences. Alternatively, the construct can include several promoter sequences each linked to a different exogenous polynucleotide sequence.

The plant cell transformed with the construct including a plurality of different exogenous polynucleotides can be regenerated into a mature plant, using the methods described hereinabove.

Alternatively, expressing a plurality of exogenous polynucleotides can be effected by introducing different nucleic acid constructs, including different exogenous polynucleotides, into a plurality of plants. The regenerated transformed plants can then be cross-bred and resultant progeny selected for superior yield or tolerance traits as described above, using conventional plant breeding techniques.

Expression of the miRNAs of the present invention or precursors thereof can be qualified using methods which are well known in the art such as those involving gene amplification e.g., PCR or RT-PCR or Northern blot or in-situ hybrdization.

According to some embodiments of the invention, the plant expressing the exogenous polynucleotide(s) is grown under stress (nitrogen or abiotic) or normal conditions (e.g., biotic conditions and/or conditions with sufficient water, nutrients such as nitrogen and fertilizer). Such conditions, which depend on the plant being grown, are known to those skilled in the art of agriculture, and are further, described above.

According to some embodiments of the invention, the method further comprises growing the plant expressing the exogenous polynucleotide(s) under abiotic stress or nitrogen limiting conditions. Non-limiting examples of abiotic stress conditions include, water deprivation, drought, excess of water (e.g., flood, waterlogging), freezing, low temperature, high temperature, strong winds, heavy metal toxicity, anaerobiosis, nutrient deficiency, nutrient excess, salinity, atmospheric pollution, intense light, insufficient light, or UV irradiation, etiolation and atmospheric pollution.

Thus, the invention encompasses plants exogenously expressing the polynucleotide(s), the nucleic acid constructs of the invention.

Methods of determining the level in the plant of the RNA transcribed from the exogenous polynucleotide are well known in the art and include, for example, Northern blot analysis, reverse transcription polymerase chain reaction (RT-PCR) analysis (including quantitative, semi-quantitative or real-time RT-PCR) and RNA-m situ hybridization.

The sequence information and annotations uncovered by the present teachings can be harnessed in favor of classical breeding. Thus, sub-sequence data of those polynucleotides described above, can be used as markers for marker assisted selection (MAS), in which a marker is used for indirect selection of a genetic determinant or determinants of a trait of interest (e.g., tolerance to abiotic stress). Nucleic acid data of the present teachings (DNA or RNA sequence) may contain or be linked to polymorphic sites or genetic markers on the genome such as restriction fragment length polymorphism (RFLP), microsatellites and single nucleotide polymorphism (SNP), DNA fingerprinting (DFP), amplified fragment length polymorphism (AFLP), expression level polymorphism, and any other polymorphism at the DNA or RNA sequence.

Examples of marker assisted selections include, but are not limited to, selection for a morphological trait (e.g., a gene that affects form, coloration, male sterility or resistance such as the presence or absence of awn, leaf sheath coloration, height, grain color, aroma of rice); selection for a biochemical trait (e.g., a gene that encodes a protein that can be extracted and observed; for example, isozymes and storage proteins); selection for a biological trait (e.g., pathogen races or insect biotypes based on host pathogen or host parasite interaction can be used as a marker since the genetic constitution of an organism can affect its susceptibility to pathogens or parasites).

The polynucleotides described hereinabove can be used in a wide range of economical plants, in a safe and cost effective manner.

Plant lines exogenously expressing the polynucleotide of the invention can be screened to identify those that show the greatest increase of the desired plant trait.

Thus, according to an additional embodiment of the present invention, there is provided a method of evaluating a trait of a plant, the method comprising: (a) expressing in a plant or a portion thereof the nucleic acid construct; and (b) evaluating a trait of a plant as compared to a wild type plant of the same type; thereby evaluating the trait of the plant.

Thus, the effect of the transgene (the exogenous polynucleotide) on different plant characteristics may be determined any method known to one of ordinary skill in the art.

Thus, for example, tolerance to limiting nitrogen conditions may be compared in transformed plants {i.e., expressing the transgene) compared to non-transformed (wild type) plants exposed to the same stress conditions (other stress conditions are contemplated as well, e.g. water deprivation, salt stress e.g. salinity, suboptimal temperatureosmotic stress, and the like), using the following assays.

Methods of qualifying plants as being tolerant or having improved tolerance to abiotic stress or limiting nitrogen levels are well known in the art and are further described hereinbelow.

Fertilizer use efficiency—To analyze whether the transgenic plants are more responsive to fertilizers, plants are grown in agar plates or pots with a limited amount of fertilizer, as described, for example, in Yanagisawa et al (Proc Natl Acad Sci USA. 2004; 101:7833-8). The plants are analyzed for their overall size, time to flowering, yield, protein content of shoot and/or grain. The parameters checked are the overall size of the mature plant, its wet and dry weight, the weight of the seeds yielded, the average seed size and the number of seeds produced per plant. Other parameters that may be tested are: the chlorophyll content of leaves (as nitrogen plant status and the degree of leaf verdure is highly correlated), amino acid and the total protein content of the seeds or other plant parts such as leaves or shoots, oil content, etc. Similarly, instead of providing nitrogen at limiting amounts, phosphate or potassium can be added at increasing concentrations. Again, the same parameters measured are the same as listed above. In this way, nitrogen use efficiency (NUE), phosphate use efficiency (PUE) and potassium use efficiency (KUE) are assessed, checking the ability of the transgenic plants to thrive under nutrient restraining conditions.

Nitrogen use efficiency—To analyze whether the transgenic plants (e.g., Arabidopsis plants) are more responsive to nitrogen, plant are grown in 0.75-3 millimolar (mM, nitrogen deficient conditions) or 10, 6-9 mM (optimal nitrogen concentration). Plants are allowed to grow for additional 25 days or until seed production. The plants are then analyzed for their overall size, time to flowering, yield, protein content of shoot and/or grain/seed production. The parameters checked can be the overall size of the plant, wet and dry weight, the weight of the seeds yielded, the average seed size and the number of seeds produced per plant. Other parameters that may be tested are: the chlorophyll content of leaves (as nitrogen plant status and the degree of leaf greenness is highly correlated), amino acid and the total protein content of the seeds or other plant parts such as leaves or shoots and oil content. Transformed plants not exhibiting substantial physiological and/or morphological effects, or exhibiting higher measured parameters levels than wild-type plants, are identified as nitrogen use efficient plants.

Nitrogen Use efficiency assay using plantlets—The assay is done according to Yanagisawa-S. et al. with minor modifications (“Metabolic engineering with Dofl transcription factor in plants: Improved nitrogen assimilation and growth under low-nitrogen conditions” Proc. Natl. Acad. Sci. USA 101, 7833-7838). Briefly, transgenic plants which are grown for 7-10 days in 0.5×MS [Murashige-Skoog] supplemented with a selection agent are transferred to two nitrogen-limiting conditions: MS media in which the combined nitrogen concentration (NH₄NO₃ and KNO₃) was 0.75 mM (nitrogen deficient conditions) or 6-15 mM (optimal nitrogen concentration). Plants are allowed to grow for additional 30-40 days and then photographed, individually removed from the Agar (the shoot without the roots) and immediately weighed (fresh weight) for later statistical analysis. Constructs for which only T1 seeds are available are sown on selective media and at least 20 seedlings (each one representing an independent transformation event) are carefully transferred to the nitrogen-limiting media. For constructs for which T2 seeds are available, different transformation events are analyzed. Usually, 20 randomly selected plants from each event are transferred to the nitrogen-limiting media allowed to grow for 3-4 additional weeks and individually weighed at the end of that period. Transgenic plants are compared to control plants grown in parallel under the same conditions. Mock-transgenic plants expressing the uidA reporter gene (GUS) under the same promoter or transgenic plants carrying the same promoter but lacking a reporter gene are used as control.

Nitrogen determination—The procedure for N (nitrogen) concentration determination in the structural parts of the plants involves the potassium persulfate digestion method to convert organic N to NO₃⁻ (Purcell and King 1996 Argon. J. 88:111-113, the modified Cd₃⁻ mediated reduction of NO₃⁻ to NO₂⁻ (Vodovotz 1996 Biotechniques 20:390-394) and the measurement of nitrite by the Griess assay (Vodovotz 1996, supra). The absorbance values are measured at 550 nm against a standard curve of NaNO₂. The procedure is described in details in Samonte et al. 2006 Agron. J. 98:168-176.

Tolerance to abiotic stress (e.g. tolerance to drought or salinity) can be evaluated by determining the differences in physiological and/or physical condition, including but not limited to, vigor, growth, size, or root length, or specifically, leaf color or leaf area size of the transgenic plant compared to a non-modified plant of the same species grown under the same conditions. Other techniques for evaluating tolerance to abiotic stress include, but are not limited to, measuring chlorophyll fluorescence, photosynthetic rates and gas exchange rates. Further assays for evaluating tolerance to abiotic stress are provided hereinbelow and in the Examples section which follows.

Drought tolerance assay—Soil-based drought screens are performed with plants overexpressing the polynucleotides detailed above. Seeds from control Arabidopsis plants, or other transgenic plants overexpressing nucleic acid of the invention are germinated and transferred to pots. Drought stress is obtained after irrigation is ceased. Transgenic and control plants are compared to each other when the majority of the control plants develop severe wilting. Plants are re-watered after obtaining a significant fraction of the control plants displaying a severe wilting. Plants are ranked comparing to controls for each of two criteria: tolerance to the drought conditions and recovery (survival) following re-watering.

Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as drought stress tolerant plants

Salinity tolerance assay—Transgenic plants with tolerance to high salt concentrations are expected to exhibit better germination, seedling vigor or growth in high salt. Salt stress can be effected in many ways such as, for example, by irrigating the plants with a hyperosmotic solution, by cultivating the plants hydroponically in a hyperosmotic growth solution (e.g., Hoagland solution with added salt), or by culturing the plants in a hyperosmotic growth medium [e.g., 50% Murashige-Skoog medium (MS medium) with added salt]. Since different plants vary considerably in their tolerance to salinity, the salt concentration in the irrigation water, growth solution, or growth medium can be adjusted according to the specific characteristics of the specific plant cultivar or variety, so as to inflict a mild or moderate effect on the physiology and/or morphology of the plants (for guidelines as to appropriate concentration see, Bernstein and Kafkafi, Root Growth Under Salinity Stress In: Plant Roots, The Hidden Half 3rd ed. Waisel Y, Eshel A and Kafkafi U. (editors) Marcel Dekker Inc., New York, 2002, and reference therein).

For example, a salinity tolerance test can be performed by irrigating plants at different developmental stages with increasing concentrations of sodium chloride (for example 50 mM, 150 mM, 300 mM NaCl) applied from the bottom and from above to ensure even dispersal of salt. Following exposure to the stress condition the plants are frequently monitored until substantial physiological and/or morphological effects appear in wild type plants. Thus, the external phenotypic appearance, degree of chlorosis and overall success to reach maturity and yield progeny are compared between control and transgenic plants. Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as abiotic stress tolerant plants.

Osmotic tolerance test—Osmotic stress assays (including sodium chloride and PEG assays) are conducted to determine if an osmotic stress phenotype was sodium chloride-specific or if it was a general osmotic stress related phenotype. Plants which are tolerant to osmotic stress may have more tolerance to drought and/or freezing. For salt and osmotic stress experiments, the medium is supplemented for example with 50 mM, 100 mM, 200 mM NaCl or 15%, 20% or 25% PEG.

Cold stress tolerance—One way to analyze cold stress is as follows. Mature (25 day old) plants are transferred to 4° C. chambers for 1 or 2 weeks, with constitutive light. Later on plants are moved back to greenhouse. Two weeks later damages from chilling period, resulting in growth retardation and other phenotypes, are compared between control and transgenic plants, by measuring plant weight (wet and dry), and by comparing growth rates measured as time to flowering, plant size, yield, and the like.

Heat stress tolerance—One way to measure heat stress tolerance is by exposing the plants to temperatures above 34° C. for a certain period. Plant tolerance is examined after transferring the plants back to 22° C. for recovery and evaluation after 5 days relative to internal controls (non-transgenic plants) or plants not exposed to neither cold or heat stress.

The biomass, vigor and yield of the plant can also be evaluated using any method known to one of ordinary skill in the art. Thus, for example, plant vigor can be calculated by the increase in growth parameters such as leaf area, fiber length, rosette diameter, plant fresh weight, oil content, seed yield and the like per time.

As mentioned, the increase of plant yield can be determined by various parameters. For example, increased yield of rice may be manifested by an increase in one or more of the following: number of plants per growing area, number of panicles per plant, number of spikelets per panicle, number of flowers per panicle, increase in the seed filling rate, increase in thousand kernel weight (1000-weight), increase oil content per seed, increase starch content per seed, among others. An increase in yield may also result in modified architecture, or may occur because of modified architecture. Similarly, increased yield of soybean may be manifested by an increase in one or more of the following: number of plants per growing area, number of pods per plant, number of seeds per pod, increase in the seed filling rate, increase in thousand seed weight (1000-weight), reduce pod shattering, increase oil content per seed, increase protein content per seed, among others. An increase in yield may also result in modified architecture, or may occur because of modified architecture.

Thus, the present invention is of high agricultural value for increasing tolerance of plants to nitrogen deficiency or abiotic stress as well as promoting the yield, biomass and vigor of commercially desired crops.

According to another embodiment of the present invention, there is provided a food or feed comprising the plants or a portion thereof of the present invention.

In a further aspect the invention, the transgenic plants of the present invention or parts thereof are comprised in a food or feed product (e.g., dry, liquid, paste). A food or feed product is any ingestible preparation containing the transgenic plants, or parts thereof, of the present invention, or preparations made from these plants. Thus, the plants or preparations are suitable for human (or animal) consumption, i.e. the transgenic plants or parts thereof are more readily digested. Feed products of the present invention further include a oil or a beverage adapted for animal consumption.

It will be appreciated that the transgenic plants, or parts thereof, of the present invention may be used directly as feed products or alternatively may be incorporated or mixed with feed products for consumption. Furthermore, the food or feed products may be processed or used as is. Exemplary feed products comprising the transgenic plants, or parts thereof, include, but are not limited to, grains, cereals, such as oats, e.g. black oats, barley, wheat, rye, sorghum, corn, vegetables, leguminous plants, especially soybeans, root vegetables and cabbage, or green forage, such as grass or hay.

It is expected that during the life of a patent maturing from this application many relevant homolog/ortholog sequences will be developed and the scope of the term polynucleotide/nucleic acid agent is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Differential Expression of miRNAs in Maize Plant Under Optimal Versus Limited Nitrogen

Experimental Procedures

Plant Material

Corn seeds were obtained from Galil seeds (Israel). Corn variety 5605 was used in all experiments. Plants were grown at 28° C. under a 16 hr light:8 hr dark regime.

Stress Induction

Corn seeds were germinated and grown on defined growth media containing either sufficient (100% N₂) or insufficient nitrogen levels (1% or 10% N₂). Seedlings aged one or two weeks were used for tissue samples for RNA analysis, as described below.

Total RNA Extraction

Total RNA of leaf or root samples from four to eight biological repeats were extracted using the mirVana™ kit (Ambion, Austin, Tex.) by pooling 3-4 plants to one biological repeat.

Microarray Design

Custom microarrays were manufactured by Agilent Technologies by in situ synthesis. The first generation microarray consisted of a total of 13619 non-redundant DNA probes, the majority of which arose from deep sequencing data and includes different small RNA molecules (i.e. miRNAs, siRNA and predicted small RNA sequences), with each probe being printed once. An in-depth analysis of the first generation microarray, which included hybridization experiments as well as structure and orientation verifications on all its small RNAs, resulted in the formation of an improved, second generation, microarray. The second generation microarray consisted of a total 4721 non-redundant DNA 45-nucleotide long probes for all known plant small RNAs, with 912 sequences (19.32%) from Sanger version 15 and the rest (3809), encompassing miRNAs (968=20.5%), siRNAs (1626=34.44%) and predicted small RNA sequences (1215=25.74%), from deep sequencing data accumulated by the inventors, with each probe being printed in triplicate.

Results

Wild type maize plants were allowed to grow at standard, optimal conditions or nitrogen deficient conditions for one or two weeks, at the end of which they were evaluated for NUE. Three to four plants from each group were used for reproducibility. Four to eight repeats were obtained for each group, and RNA was extracted from leaf or root tissue. The expression level of the maize miRNAs was analyzed by high throughput microarray to identify miRNAs that were differentially expressed between the experimental groups.

Tables 1-2 below presents sequences that were found to be differentially expressed in corn grown in various nitrogen levels. To clarify, the sequence of an up-regulated miRNA is induced under nitrogen limiting conditions and the sequence of a down-regulated miRNA is repressed under nitrogen limiting conditions compared to optimal conditions.

TABLE 1
Differentially Expressed miRNAs in Leaf of Plants Growing
under Nitrogen Deficient Versus Optimal Conditions.

| | Fold | | | |
|---|---|---|---|---|
| P value - | Change - | | Up/Down | |
| Leaf | Leaf | Sequence/SEQ ID NO: | regulated | Small RNA name |
| 3.90E−03 | 1.66 | AGAAGAGAGAGAGTACAGCCT/1 | Down | Zma-miR529 |
| 3.30E−06 | 3.35 | TAGCCAGGGATGATTTGCCTG/2 | Down | Zma-miR1691 |
| ND | ND | GGAATCTTGATGATGCTGCAT/3 | Down | Zma-miR172e |
| ND | ND | GTGAAGTGTTTGGGGGAACTC/4 | Down | Zma-miR395b |
| 2.20E−07 | 2.51 | TAGCCAAGCATGATTTGCCCG/5 | Down | Predicted zma mir |
| | | | | 50601 |
| ND | ND | AGGATGTGAGGCTATTGGGGAC/6 | Up | Predicted zma mir |
| | | | | 48492 |
| ND | ND | CCAAGTCGAGGGCAGACCAGGC/7 | Up | Predicted zma mir |
| | | | | 48879 |
| ND | ND | ATTCACGGGGACGAACCTCCT/8 | Up | Mtr-miR2647a |
| 1.80E−02 | 1.72 | AGGATGCTGACGCAATGGGAT/9 | Up | Predicted zma mir |
| | | | | 48486 |
| 9.80E−03 | 1.61 | TTAGATGACCATCAGCAAACA/10 | Up | Zma-miR827 |

TABLE 2
Differentially Expressed miRNAs in Roots of Plants Growing
under Nitrogen Deficient Versus Optimal Conditions.

| | Fold | | | |
|---|---|---|---|---|
| P value - | Change - | | Up/Down | |
| Root | Root | Sequence/SEQ ID NO: | regulated | Small RNA name |
| ND | ND | AGAAGAGAGAGAGTACAGCCT/1 | Down | Zma-miR529 |
| 1.40E−05 | 2.56 | TAGCCAGGGATGATTTGCCTG/2 | Down | Zma-miR1691 |
| 5.40E−05 | 2.08 | GTGAAGTGTTTGGGGGAACTC/4 | Down | Zma-miR395b |
| 2.30E−04 | 1.66 | TAGCCAAGCATGATTTGCCCG/5 | Down | Predicted zma mir |
| | | | | 50601 |
| 4.50E−02 | 1.75 | GGAATCTTGATGATGCTGCAT/3 | Down | Zma-miR172e |
| 1.60E−02 | 1.8 | AGGATGCTGACGCAATGGGAT/9 | Up | Predicted zma mir |
| | | | | 48486 |
| ND | ND | TTAGATGACCATCAGCAAACA/10 | Up | Zma-miR827 |
| 1.30E−04 | 2.75 | AGGATGTGAGGCTATTGGGGAC/6 | Up | Predicted zma mir |
| | | | | 48492 |
| 5.60E−04 | 1.95 | CCAAGTCGAGGGCAGACCAGGC/7 | Up | Predicted zma mir |
| | | | | 48879 |
| 3.90E−02 | 1.79 | ATTCACGGGGACGAACCTCCT/8 | Up | Mtr-miR2647a |

### Example 2

### Identification of Homologous and Orthologous Sequences of Differential Small RNAs Associated with Increased NUE

The small RNA sequences of the invention that were either down- or upregulated under nitrogen limiting conditions were examined for homologous and orthologous sequences using the miRBase database (wwwdotmirbasedotorg/) and the Plant MicroRNA Database (PMRD, wwwdotbioinformaticsdotcaudotedudotcn/PMRD). The mature miRNA sequences that are homologous or orthologous to the miRNAs of the invention (listed in Table 1) are found using miRNA public databases, having at least 90% identity of the entire small RNA length, and are summarized in Table 3 below. Of note, if homologs of only 90% are uncovered, they are subject for family members search and are listed with a cutoff of 80% identity to the homolog sequence, not to the original maize miR.

TABLE 3
Summary of Homologs/Orthologs to NUE small RNA Probes
(upregulated)

| Homolog | | | | | Stem- | | | |
|---|---|---|---|---|---|---|---|---|
| Stem- | | | | | loop | | | |
| loop seq | % | Homolog | | Homolog | seq id | MiR | Mature | MiR |
| id no: | Identity | length | sequence | names | no: | length | sequence | Name |
| 38 | 1 | 21 | ATTCACGG | mtr- | 21 | 21 | ATTCAC | mtr- |
| | | | GGACGAAC | miR2647b | | | GGGGAC | miR2647a |
| | | | CTCCT/23 | | | | GAACCT | |
| | | | | | | | CCT/8 | |
| 39 | 1 | 21 | ATTCACGG | mtr- | | | | |
| | | | GGACGAAC | miR2647c | | | | |
| | | | CTCCT/24 | | | | | |
| 40 | 0.9 | 21 | TTAGATGA | aly- | 22 | 21 | TTAGAT | zma- |
| | | | CCATCAAC | miR827 | | | GACCAT | miR827 |
| | | | AAACG/25 | | | | CAGCAA | |
| | | | | | | | ACA/10 | |
| 41 | 0.9 | 21 | TTAGATGA | ath- | | | | |
| | | | CCATCAAC | miR827 | | | | |
| | | | AAACT/26 | | | | | |
| 42 | 1 | 21 | TTAGATGA | bdi- | | | | |
| | | | CCATCAGC | miR827 | | | | |
| | | | AAACA/27 | | | | | |
| 43 | 0.95 | 21 | TTAGATGA | csi- | | | | |
| | | | CCATCAAC | miR827 | | | | |
| | | | AAACA/28 | | | | | |
| 44 | 0.95 | 21 | TTAGATGA | ghr- | | | | |
| | | | CCATCAAC | miR827a | | | | |
| | | | AAACA/29 | | | | | |
| 45 | 0.95 | 21 | TTAGATGA | ghr- | | | | |
| | | | CCATCAAC | miR827b | | | | |
| | | | AAACA/30 | | | | | |
| 46 | 0.95 | 21 | TTAGATGA | ghr- | | | | |
| | | | CCATCAAC | miR827c | | | | |
| | | | AAACA/31 | | | | | |
| 47 | 0.86 | 21 | TAAGATGA | osa- | | | | |
| | | | CCATCAGC | miR827 | | | | |
| | | | GAAAA/32 | | | | | |
| 48 | 1 | 21 | TTAGATGA | osa- | | | | |
| | | | CCATCAGC | miR827a | | | | |
| | | | AAACA/33 | | | | | |
| 49 | 1 | 21 | TTAGATGA | osa- | | | | |
| | | | CCATCAGC | miR827b | | | | |
| | | | AAACA/34 | | | | | |
| 50 | 0.86 | 21 | TTAGATGA | ptc- | | | | |
| | | | CCATCAAC | miR827 | | | | |
| | | | GAAAA/35 | | | | | |
| 51 | 1 | 21 | TTAGATGA | ssp- | | | | |
| | | | CCATCAGC | miR827 | | | | |
| | | | AAACA/36 | | | | | |
| 52 | 0.95 | 21 | TTAGATGA | tcc- | | | | |
| | | | CCATCAAC | miR827 | | | | |
| | | | AAACA/37 | | | | | |

TABLE 4
Summary of Homologs/Orthologs to NUE small RNA Probes
(Downregulated)

| | | | | | Stem- | | | |
|---|---|---|---|---|---|---|---|---|
| | | | | | loop | | | |
| Homolog | | | | | seq | | | |
| Stem-loop | % | Homolog | | Homolog | id | MiR | Mature | MiR |
| seq id no: | Identity | length | sequence | names | no: | length | sequence | Name |
| 454 | 0.81 | 21 | CAGCCAAG | aly- | 53 | 21 | TAGCCA | zma- |
| | | | GATGACTT | miR169b | | | GGGATG | miR1691 |
| | | | GCCGG/57 | | | | ATTTGCC | |
| | | | | | | | TG/2 | |
| 455 | 0.81 | 21 | CAGCCAAG | aly- | | | | |
| | | | GATGACTT | miR169c | | | | |
| | | | GCCGG/58 | | | | | |
| 456 | 0.9 | 21 | TAGCCAAG | aly- | | | | |
| | | | GATGACTT | miR169h | | | | |
| | | | GCCTG/59 | | | | | |
| 457 | 0.9 | 21 | TAGCCAAG | aly- | | | | |
| | | | GATGACTT | miR169i | | | | |
| | | | GCCTG/60 | | | | | |
| 458 | 0.9 | 21 | TAGCCAAG | aly- | | | | |
| | | | GATGACTT | miR169j | | | | |
| | | | GCCTG/61 | | | | | |
| 459 | 0.9 | 21 | TAGCCAAG | aly- | | | | |
| | | | GATGACTT | miR169k | | | | |
| | | | GCCTG/62 | | | | | |
| 460 | 0.9 | 21 | TAGCCAAG | aly- | | | | |
| | | | GATGACTT | miR169l | | | | |
| | | | GCCTG/63 | | | | | |
| 461 | 0.9 | 21 | TAGCCAAG | aly- | | | | |
| | | | GATGACTT | miR169m | | | | |
| | | | GCCTG/64 | | | | | |
| 462 | 0.86 | 21 | TAGCCAAA | aly- | | | | |
| | | | GATGACTT | miR169n | | | | |
| | | | GCCTG/65 | | | | | |
| 463 | 0.86 | 21 | TAGCCAAG | aqc- | | | | |
| | | | GATGACTT | miR169a | | | | |
| | | | GCCTA/66 | | | | | |
| 464 | 0.9 | 21 | TAGCCAAG | aqc- | | | | |
| | | | GATGACTT | miR169b | | | | |
| | | | GCCTG/67 | | | | | |
| 465 | 0.81 | 21 | CAGCCAAG | aqc- | | | | |
| | | | GATGACTT | miR169c | | | | |
| | | | GCCGG/68 | | | | | |
| 466 | 0.86 | 21 | TAGCCAAG | ata- | | | | |
| | | | GATGAATT | miR169 | | | | |
| | | | GCCAG/69 | | | | | |
| 467 | 0.81 | 21 | CAGCCAAG | ath- | | | | |
| | | | GATGACTT | miR169b | | | | |
| | | | GCCGG/70 | | | | | |
| 468 | 0.81 | 21 | CAGCCAAG | ath- | | | | |
| | | | GATGACTT | miR169c | | | | |
| | | | GCCGG/71 | | | | | |
| 469 | 0.9 | 21 | TAGCCAAG | ath- | | | | |
| | | | GATGACTT | miR169h | | | | |
| | | | GCCTG/72 | | | | | |
| 470 | 0.9 | 21 | TAGCCAAG | ath- | | | | |
| | | | GATGACTT | miR169i | | | | |
| | | | GCCTG/73 | | | | | |
| 471 | 0.9 | 21 | TAGCCAAG | ath- | | | | |
| | | | GATGACTT | miR169j | | | | |
| | | | GCCTG/74 | | | | | |
| 472 | 0.9 | 21 | TAGCCAAG | ath- | | | | |
| | | | GATGACTT | miR169k | | | | |
| | | | GCCTG/75 | | | | | |
| 473 | 0.9 | 21 | TAGCCAAG | ath- | | | | |
| | | | GATGACTT | miR169l | | | | |
| | | | GCCTG/76 | | | | | |
| 474 | 0.9 | 21 | TAGCCAAG | ath- | | | | |
| | | | GATGACTT | miR169m | | | | |
| | | | GCCTG/77 | | | | | |
| 475 | 0.9 | 21 | TAGCCAAG | ath- | | | | |
| | | | GATGACTT | miR169n | | | | |
| | | | GCCTG/78 | | | | | |
| 476 | 0.86 | 21 | TAGCCAAG | bdi- | | | | |
| | | | GATGACTT | miR169b | | | | |
| | | | GCCGG/79 | | | | | |
| 477 | 0.81 | 21 | CAGCCAAG | bdi- | | | | |
| | | | GATGACTT | miR169c | | | | |
| | | | GCCGG/80 | | | | | |
| 478 | 0.81 | 21 | TAGCCAAG | bdi- | | | | |
| | | | AATGACTT | miR169d | | | | |
| | | | GCCTA/81 | | | | | |
| 479 | 0.9 | 21 | TAGCCAAG | bdi- | | | | |
| | | | GATGACTT | miR169e | | | | |
| | | | GCCTG/82 | | | | | |
| 480 | 0.81 | 21 | CAGCCAAG | bdi- | | | | |
| | | | GATGACTT | miR169f | | | | |
| | | | GCCGG/83 | | | | | |
| 481 | 0.9 | 21 | TAGCCAAG | bdi- | | | | |
| | | | GATGACTT | miR169g | | | | |
| | | | GCCTG/84 | | | | | |
| 482 | 0.86 | 21 | TAGCCAAG | bdi- | | | | |
| | | | GATGACTT | miR169h | | | | |
| | | | GCCTA/85 | | | | | |
| 483 | 0.81 | 21 | TAGCCAGG | bdi- | | | | |
| | | | AATGGCTT | miR169j | | | | |
| | | | GCCTA/86 | | | | | |
| 484 | 0.95 | 22 | TAGCCAAG | bdi- | | | | |
| | | | GATGATTT | miR169k | | | | |
| | | | GCCTGT/87 | | | | | |
| 485 | 0.86 | 21 | TAGCCAAG | bna- | | | | |
| | | | GATGACTT | miR169c | | | | |
| | | | GCCTA/88 | | | | | |
| 486 | 0.86 | 21 | TAGCCAAG | bna- | | | | |
| | | | GATGACTT | miR169d | | | | |
| | | | GCCTA/89 | | | | | |
| 487 | 0.86 | 21 | TAGCCAAG | bna- | | | | |
| | | | GATGACTT | miR169e | | | | |
| | | | GCCTA/90 | | | | | |
| 488 | 0.86 | 21 | TAGCCAAG | bna- | | | | |
| | | | GATGACTT | miR169f | | | | |
| | | | GCCTA/91 | | | | | |
| 489 | 0.9 | 22 | TAGCCAAG | bna- | | | | |
| | | | GATGACTT | miR169g | | | | |
| | | | GCCTGC/92 | | | | | |
| 490 | 0.9 | 22 | TAGCCAAG | bna- | | | | |
| | | | GATGACTT | miR169h | | | | |
| | | | GCCTGC/93 | | | | | |
| 491 | 0.9 | 22 | TAGCCAAG | bna- | | | | |
| | | | GATGACTT | miR169i | | | | |
| | | | GCCTGC/94 | | | | | |
| 492 | 0.9 | 22 | TAGCCAAG | bna- | | | | |
| | | | GATGACTT | miR169j | | | | |
| | | | GCCTGC/95 | | | | | |
| 493 | 0.9 | 22 | TAGCCAAG | bna- | | | | |
| | | | GATGACTT | miR169k | | | | |
| | | | GCCTGC/96 | | | | | |
| 494 | 0.9 | 22 | TAGCCAAG | bna- | | | | |
| | | | GATGACTT | miR169l | | | | |
| | | | GCCTGC/97 | | | | | |
| 495 | 0.86 | 21 | TAGCCAAG | far- | | | | |
| | | | GATGACTT | miR169 | | | | |
| | | | GCCTA/98 | | | | | |
| 496 | 0.9 | 21 | TAGCCAAG | ghb- | | | | |
| | | | GATGACTT | miR169a | | | | |
| | | | GCCTG/99 | | | | | |
| 497 | 0.81 | 21 | CAGCCAAG | gma- | | | | |
| | | | GATGACTT | miR169a | | | | |
| | | | GCCGG/100 | | | | | |
| 498 | 0.81 | 23 | TGAGCCAA | gma- | | | | |
| | | | GGATGACT | miR169d | | | | |
| | | | TGCCGGT/101 | | | | | |
| 499 | 0.81 | 20 | AGCCAAGG | gma- | | | | |
| | | | ATGACTTG | miR169e | | | | |
| | | | CCGG/102 | | | | | |
| 500 | 0.86 | 21 | AAGCCAAG | hvu- | | | | |
| | | | GATGAGTT | miR169 | | | | |
| | | | GCCTG/103 | | | | | |
| 501 | 0.81 | 21 | CAGCCAAG | mtr- | | | | |
| | | | GGTGATTT | miR169c | | | | |
| | | | GCCGG/104 | | | | | |
| 502 | 0.81 | 21 | AAGCCAAG | mtr- | | | | |
| | | | GATGACTT | miR169d | | | | |
| | | | GCCGG/105 | | | | | |
| 503 | 0.81 | 21 | AAGCCAAG | mtr- | | | | |
| | | | GATGACTT | miR169f | | | | |
| | | | GCCTA/106 | | | | | |
| 504 | 0.81 | 21 | CAGCCAAG | mtr- | | | | |
| | | | GATGACTT | miR169g | | | | |
| | | | GCCGG/107 | | | | | |
| 505 | 0.81 | 21 | CAGCCAAG | mtr- | | | | |
| | | | GATGACTT | miR169j | | | | |
| | | | GCCGG/108 | | | | | |
| 506 | 0.81 | 21 | CAGCCAAG | mtr- | | | | |
| | | | GGTGATTT | miR169k | | | | |
| | | | GCCGG/109 | | | | | |
| 507 | 0.81 | 21 | AAGCCAAG | mtr- | | | | |
| | | | GATGACTT | miR169l | | | | |
| | | | GCCGG/110 | | | | | |
| 508 | 0.81 | 21 | GAGCCAAG | mtr- | | | | |
| | | | GATGACTT | miR169m | | | | |
| | | | GCCGG/111 | | | | | |
| 509 | 0.81 | 21 | CAGCCAAG | osa- | | | | |
| | | | GATGACTT | miR169b | | | | |
| | | | GCCGG/112 | | | | | |
| 510 | 0.81 | 21 | CAGCCAAG | osa- | | | | |
| | | | GATGACTT | miR169c | | | | |
| | | | GCCGG/113 | | | | | |
| 511 | 0.86 | 21 | TAGCCAAG | osa- | | | | |
| | | | GATGAATT | miR169d | | | | |
| | | | GCCGG/114 | | | | | |
| 512 | 0.86 | 21 | TAGCCAAG | osa- | | | | |
| | | | GATGACTT | miR169e | | | | |
| | | | GCCGG/115 | | | | | |
| 513 | 0.86 | 21 | TAGCCAAG | osa- | | | | |
| | | | GATGACTT | miR169f | | | | |
| | | | GCCTA/116 | | | | | |
| 514 | 0.86 | 21 | TAGCCAAG | osa- | | | | |
| | | | GATGACTT | miR169g | | | | |
| | | | GCCTA/117 | | | | | |
| 515 | 0.9 | 21 | TAGCCAAG | osa- | | | | |
| | | | GATGACTT | miR169h | | | | |
| | | | GCCTG/118 | | | | | |
| 516 | 0.9 | 21 | TAGCCAAG | osa- | | | | |
| | | | GATGACTT | miR169i | | | | |
| | | | GCCTG/119 | | | | | |
| 517 | 0.9 | 21 | TAGCCAAG | osa- | | | | |
| | | | GATGACTT | miR169j | | | | |
| | | | GCCTG/120 | | | | | |
| 518 | 0.9 | 21 | TAGCCAAG | osa- | | | | |
| | | | GATGACTT | miR169k | | | | |
| | | | GCCTG/121 | | | | | |
| 519 | 0.9 | 21 | TAGCCAAG | osa- | | | | |
| | | | GATGACTT | miR169l | | | | |
| | | | GCCTG/122 | | | | | |
| 520 | 0.9 | 21 | TAGCCAAG | osa- | | | | |
| | | | GATGACTT | miR169m | | | | |
| | | | GCCTG/123 | | | | | |
| 521 | 0.81 | 21 | TAGCCAAG | osa- | | | | |
| | | | AATGACTT | miR169n | | | | |
| | | | GCCTA/124 | | | | | |
| 522 | 0.81 | 21 | TAGCCAAG | osa- | | | | |
| | | | AATGACTT | miR169o | | | | |
| | | | GCCTA/125 | | | | | |
| 523 | 0.81 | 21 | CAGCCAAG | ptc- | | | | |
| | | | GATGACTT | miR169d | | | | |
| | | | GCCGG/126 | | | | | |
| 524 | 0.81 | 21 | CAGCCAAG | ptc- | | | | |
| | | | GATGACTT | miR169e | | | | |
| | | | GCCGG/127 | | | | | |
| 525 | 0.81 | 21 | CAGCCAAG | ptc- | | | | |
| | | | GATGACTT | miR169f | | | | |
| | | | GCCGG/128 | | | | | |
| 526 | 0.81 | 21 | CAGCCAAG | ptc- | | | | |
| | | | GATGACTT | miR169g | | | | |
| | | | GCCGG/129 | | | | | |
| 527 | 0.81 | 21 | CAGCCAAG | ptc- | | | | |
| | | | GATGACTT | miR169h | | | | |
| | | | GCCGG/130 | | | | | |
| 528 | 0.9 | 21 | TAGCCAAG | ptc- | | | | |
| | | | GATGACTT | miR169i | | | | |
| | | | GCCTG/131 | | | | | |
| 529 | 0.9 | 21 | TAGCCAAG | ptc- | | | | |
| | | | GATGACTT | miR169j | | | | |
| | | | GCCTG/132 | | | | | |
| 530 | 0.9 | 21 | TAGCCAAG | ptc- | | | | |
| | | | GATGACTT | miR169k | | | | |
| | | | GCCTG/133 | | | | | |
| 531 | 0.9 | 21 | TAGCCAAG | ptc- | | | | |
| | | | GATGACTT | miR169l | | | | |
| | | | GCCTG/134 | | | | | |
| 532 | 0.9 | 21 | TAGCCAAG | ptc- | | | | |
| | | | GATGACTT | miR169m | | | | |
| | | | GCCTG/135 | | | | | |
| 533 | 0.86 | 21 | AAGCCAAG | ptc- | | | | |
| | | | GATGACTT | miR169o | | | | |
| | | | GCCTG/136 | | | | | |
| 534 | 0.86 | 21 | AAGCCAAG | ptc- | | | | |
| | | | GATGACTT | miR169p | | | | |
| | | | GCCTG/137 | | | | | |
| 535 | 0.86 | 21 | TAGCCAAG | ptc- | | | | |
| | | | GACGACTT | miR169q | | | | |
| | | | GCCTG/138 | | | | | |
| 536 | 0.86 | 21 | TAGCCAAG | ptc- | | | | |
| | | | GATGACTT | miR169r | | | | |
| | | | GCCTA/139 | | | | | |
| 537 | 0.81 | 21 | TAGCCAAG | ptc- | | | | |
| | | | GACGACTT | miR169u | | | | |
| | | | GCCTA/140 | | | | | |
| 538 | 0.81 | 21 | TAGCCAAG | ptc- | | | | |
| | | | GATGACTT | miR169v | | | | |
| | | | GCCCA/141 | | | | | |
| 539 | 0.81 | 21 | TAGCCAAG | ptc- | | | | |
| | | | GATGACTT | miR169w | | | | |
| | | | GCCCA/142 | | | | | |
| 540 | 0.81 | 21 | TAGCCAAG | ptc- | | | | |
| | | | GATGACTT | miR169x | | | | |
| | | | GCTCG/143 | | | | | |
| 541 | 0.9 | 21 | TAGCCATG | ptc- | | | | |
| | | | GATGAATT | miR169y | | | | |
| | | | GCCTG/144 | | | | | |
| 542 | 0.81 | 21 | CAGCCAAG | ptc- | | | | |
| | | | AATGATTT | miR169z | | | | |
| | | | GCCGG/145 | | | | | |
| 543 | 0.81 | 21 | CAGCCAAG | rco- | | | | |
| | | | GATGACTT | miR169a | | | | |
| | | | GCCGG/146 | | | | | |
| 544 | 0.81 | 21 | CAGCCAAG | rco- | | | | |
| | | | GATGACTT | miR169b | | | | |
| | | | GCCGG/147 | | | | | |
| 545 | 0.81 | 21 | CAGCCAAG | sbi- | | | | |
| | | | GATGACTT | miR169b | | | | |
| | | | GCCGG/148 | | | | | |
| 546 | 0.86 | 21 | TAGCCAAG | sbi- | | | | |
| | | | GATGACTT | miR169c | | | | |
| | | | GCCTA/149 | | | | | |
| 547 | 0.86 | 21 | TAGCCAAG | sbi- | | | | |
| | | | GATGACTT | miR169d | | | | |
| | | | GCCTA/150 | | | | | |
| 548 | 0.86 | 21 | TAGCCAAG | sbi- | | | | |
| | | | GATGACTT | miR169e | | | | |
| | | | GCCGG/151 | | | | | |
| 549 | 0.9 | 21 | TAGCCAAG | sbi- | | | | |
| | | | GATGACTT | miR169f | | | | |
| | | | GCCTG/152 | | | | | |
| 550 | 0.9 | 21 | TAGCCAAG | sbi- | | | | |
| | | | GATGACTT | miR169g | | | | |
| | | | GCCTG/153 | | | | | |
| 551 | 0.86 | 21 | TAGCCAAG | sbi- | | | | |
| | | | GATGACTT | miR169h | | | | |
| | | | GCCTA/154 | | | | | |
| 552 | 0.81 | 21 | TAGCCAAG | sbi- | | | | |
| | | | AATGACTT | miR169i | | | | |
| | | | GCCTA/155 | | | | | |
| 553 | 0.86 | 21 | TAGCCAAG | sbi- | | | | |
| | | | GATGACTT | miR169j | | | | |
| | | | GCCGG/156 | | | | | |
| 554 | 0.81 | 21 | CAGCCAAG | sbi- | | | | |
| | | | GATGACTT | miR169k | | | | |
| | | | GCCGG/157 | | | | | |
| 555 | 0.9 | 21 | TAGCCAAG | sbi- | | | | |
| | | | GATGACTT | miR169l | | | | |
| | | | GCCTG/158 | | | | | |
| 556 | 0.86 | 21 | TAGCCAAG | sbi- | | | | |
| | | | GATGACTT | miR169m | | | | |
| | | | GCCTA/159 | | | | | |
| 557 | 0.86 | 21 | TAGCCAAG | sbi- | | | | |
| | | | GATGACTT | miR169n | | | | |
| | | | GCCTA/160 | | | | | |
| 558 | 0.95 | 21 | TAGCCAAG | sbi- | | | | |
| | | | GATGATTT | miR169o | | | | |
| | | | GCCTG/161 | | | | | |
| 559 | 0.81 | 21 | CAGCCAAG | sly- | | | | |
| | | | GATGACTT | miR169a | | | | |
| | | | GCCGG/162 | | | | | |
| 560 | 0.9 | 21 | TAGCCAAG | sly- | | | | |
| | | | GATGACTT | miR169b | | | | |
| | | | GCCTG/163 | | | | | |
| 561 | 0.86 | 21 | TAGCCAAG | sly- | | | | |
| | | | GATGACTT | miR169d | | | | |
| | | | GCCTA/164 | | | | | |
| 562 | 0.86 | 21 | TAGCCAAG | ssp- | | | | |
| | | | GATGACTT | miR169 | | | | |
| | | | GCCGG/165 | | | | | |
| 563 | 0.81 | 21 | CAGCCAAG | tcc- | | | | |
| | | | GATGACTT | miR169b | | | | |
| | | | GCCGG/166 | | | | | |
| 564 | 0.86 | 21 | TAGCCAAG | tcc- | | | | |
| | | | GATGACTT | miR169d | | | | |
| | | | GCCTA/167 | | | | | |
| 565 | 0.81 | 21 | AAGCCAAG | tcc- | | | | |
| | | | AATGACTT | miR169f | | | | |
| | | | GCCTG/168 | | | | | |
| 566 | 0.9 | 21 | TAGCCAGG | tcc- | | | | |
| | | | GATGACTT | miR169g | | | | |
| | | | GCCTA/169 | | | | | |
| 567 | 0.9 | 21 | TAGCCAAG | tcc- | | | | |
| | | | GATGACTT | miR169h | | | | |
| | | | GCCTG/170 | | | | | |
| 568 | 0.9 | 21 | TAGCCAAG | tcc- | | | | |
| | | | GATGAGTT | miR169i | | | | |
| | | | GCCTG/171 | | | | | |
| 569 | 0.9 | 21 | TAGCCAAG | tcc- | | | | |
| | | | GATGACTT | miR169j | | | | |
| | | | GCCTG/172 | | | | | |
| 570 | 0.81 | 21 | CAGCCAAG | tcc- | | | | |
| | | | GATGACTT | miR169k | | | | |
| | | | GCCGG/173 | | | | | |
| 571 | 0.81 | 21 | CAGCCAAG | tcc- | | | | |
| | | | GATGACTT | miR169l | | | | |
| | | | GCCGG/174 | | | | | |
| 572 | 0.81 | 21 | CAGCCAAG | vvi- | | | | |
| | | | GATGACTT | miR169a | | | | |
| | | | GCCGG/175 | | | | | |
| 573 | 0.81 | 21 | CAGCCAAG | vvi- | | | | |
| | | | GATGACTT | miR169c | | | | |
| | | | GCCGG/176 | | | | | |
| 574 | 0.81 | 21 | CAGCCAAG | vvi- | | | | |
| | | | AATGATTT | miR169d | | | | |
| | | | GCCGG/177 | | | | | |
| 575 | 0.9 | 22 | TAGCCAAG | vvi- | | | | |
| | | | GATGACTT | miR169e | | | | |
| | | | GCCTGC/178 | | | | | |
| 576 | 0.81 | 21 | CAGCCAAG | vvi- | | | | |
| | | | GATGACTT | miR169j | | | | |
| | | | GCCGG/179 | | | | | |
| 577 | 0.81 | 21 | CAGCCAAG | vvi- | | | | |
| | | | GATGACTT | miR169k | | | | |
| | | | GCCGG/180 | | | | | |
| 578 | 0.81 | 21 | GAGCCAAG | vvi- | | | | |
| | | | GATGACTT | miR169m | | | | |
| | | | GCCGG/181 | | | | | |
| 579 | 0.81 | 21 | GAGCCAAG | vvi- | | | | |
| | | | GATGACTT | miR169n | | | | |
| | | | GCCGG/182 | | | | | |
| 580 | 0.81 | 21 | GAGCCAAG | vvi- | | | | |
| | | | GATGACTT | miR169p | | | | |
| | | | GCCGG/183 | | | | | |
| 581 | 0.81 | 21 | GAGCCAAG | vvi- | | | | |
| | | | GATGACTT | miR169q | | | | |
| | | | GCCGG/184 | | | | | |
| 582 | 0.81 | 21 | CAGCCAAG | vvi- | | | | |
| | | | GATGACTT | miR169s | | | | |
| | | | GCCGG/185 | | | | | |
| 583 | 0.81 | 21 | AAGCCAAG | vvi- | | | | |
| | | | GATGAATT | miR169v | | | | |
| | | | GCCGG/186 | | | | | |
| 584 | 0.81 | 21 | CAGCCAAG | vvi- | | | | |
| | | | GATGACTT | miR169w | | | | |
| | | | GCCGG/187 | | | | | |
| 585 | 0.86 | 21 | TAGCCAAG | vvi- | | | | |
| | | | GATGACTT | miR169x | | | | |
| | | | GCCTA/188 | | | | | |
| 586 | 0.81 | 21 | TAGCGAAG | vvi- | | | | |
| | | | GATGACTT | miR169y | | | | |
| | | | GCCTA/189 | | | | | |
| 587 | 0.81 | 21 | CAGCCAAG | zma- | | | | |
| | | | GATGACTT | miR169c | | | | |
| | | | GCCGG/190 | | | | | |
| 588 | 0.86 | 21 | TAGCCAAG | zma- | | | | |
| | | | GATGACTT | miR169f | | | | |
| | | | GCCTA/191 | | | | | |
| 589 | 0.86 | 21 | TAGCCAAG | zma- | | | | |
| | | | GATGACTT | miR169g | | | | |
| | | | GCCTA/192 | | | | | |
| 590 | 0.86 | 21 | TAGCCAAG | zma- | | | | |
| | | | GATGACTT | miR169h | | | | |
| | | | GCCTA/193 | | | | | |
| 591 | 0.9 | 21 | TAGCCAAG | zma- | | | | |
| | | | GATGACTT | miR169i | | | | |
| | | | GCCTG/194 | | | | | |
| 592 | 0.9 | 21 | TAGCCAAG | zma- | | | | |
| | | | GATGACTT | miR169j | | | | |
| | | | GCCTG/195 | | | | | |
| 593 | 0.9 | 21 | TAGCCAAG | zma- | | | | |
| | | | GATGACTT | miR169k | | | | |
| | | | GCCTG/196 | | | | | |
| 594 | 0.81 | 21 | TAGCCAAG | zma- | | | | |
| | | | AATGACTT | miR169o | | | | |
| | | | GCCTA/197 | | | | | |
| 595 | 0.86 | 21 | TAGCCAAG | zma- | | | | |
| | | | GATGACTT | miR169p | | | | |
| | | | GCCGG/198 | | | | | |
| 596 | 0.81 | 21 | CAGCCAAG | zma- | | | | |
| | | | GATGACTT | miR169r | | | | |
| | | | GCCGG/199 | | | | | |
| 597 | 0.95 | 21 | AGAATCTT | aly- | 54 | 21 | GGAATC | zma- |
| | | | GATGATGC | miR172a | | | TTGATG | miR172e |
| | | | TGCAT/200 | | | | ATGCTG | |
| | | | | | | | CAT/3 | |
| 598 | 0.95 | 21 | AGAATCTT | aly- | | | | |
| | | | GATGATGC | miR172b | | | | |
| | | | TGCAT/201 | | | | | |
| 599 | 0.9 | 21 | AGAATCTT | aly- | | | | |
| | | | GATGATGC | miR172c | | | | |
| | | | TGCAG/202 | | | | | |
| 600 | 0.9 | 21 | AGAATCTT | aly- | | | | |
| | | | GATGATGC | miR172d | | | | |
| | | | TGCAG/203 | | | | | |
| 601 | 0.95 | 20 | GAATCTTG | aly- | | | | |
| | | | ATGATGCT | miR172e | | | | |
| | | | GCAT/204 | | | | | |
| 602 | 0.95 | 21 | AGAATCTT | aqc- | | | | |
| | | | GATGATGC | miR172a | | | | |
| | | | TGCAT/205 | | | | | |
| 603 | 1 | 21 | GGAATCTT | aqc- | | | | |
| | | | GATGATGC | miR172b | | | | |
| | | | TGCAT/206 | | | | | |
| 604 | 0.86 | 21 | AGGATCTT | asp- | | | | |
| | | | GATGATGC | miR172 | | | | |
| | | | TGCAG/207 | | | | | |
| 605 | 0.95 | 23 | TGAGAATC | ata- | | | | |
| | | | TTGATGAT | miR172 | | | | |
| | | | GCTGCAT/208 | | | | | |
| 606 | 0.95 | 21 | AGAATCTT | ath- | | | | |
| | | | GATGATGC | miR172a | | | | |
| | | | TGCAT/209 | | | | | |
| 607 | 0.95 | 21 | AGAATCTT | ath- | | | | |
| | | | GATGATGC | miR172b | | | | |
| | | | TGCAT/210 | | | | | |
| 608 | 0.9 | 21 | AGAATCTT | ath- | | | | |
| | | | GATGATGC | miR172c | | | | |
| | | | TGCAG/211 | | | | | |
| 609 | 0.9 | 21 | AGAATCTT | ath- | | | | |
| | | | GATGATGC | miR172d | | | | |
| | | | TGCAG/212 | | | | | |
| 610 | 1 | 21 | GGAATCTT | ath- | | | | |
| | | | GATGATGC | miR172e | | | | |
| | | | TGCAT/213 | | | | | |
| 611 | 0.86 | 21 | AGAATCCT | ath- | | | | |
| | | | GATGATGC | miR172m | | | | |
| | | | TGCAG/214 | | | | | |
| 612 | 0.95 | 21 | AGAATCTT | bdi- | | | | |
| | | | GATGATGC | miR172a | | | | |
| | | | TGCAT/215 | | | | | |
| 613 | 1 | 21 | GGAATCTT | bdi- | | | | |
| | | | GATGATGC | miR172b | | | | |
| | | | TGCAT/216 | | | | | |
| 614 | 0.86 | 21 | AGAATCCT | bdi- | | | | |
| | | | GATGATGC | miR172d | | | | |
| | | | TGCAG/217 | | | | | |
| 615 | 0.95 | 21 | AGAATCTT | bol- | | | | |
| | | | GATGATGC | miR172a | | | | |
| | | | TGCAT/218 | | | | | |
| 616 | 0.95 | 21 | AGAATCTT | bol- | | | | |
| | | | GATGATGC | miR172b | | | | |
| | | | TGCAT/219 | | | | | |
| 617 | 0.95 | 21 | AGAATCTT | bra- | | | | |
| | | | GATGATGC | miR172a | | | | |
| | | | TGCAT/220 | | | | | |
| 618 | 0.95 | 21 | AGAATCTT | bra- | | | | |
| | | | GATGATGC | miR172b | | | | |
| | | | TGCAT/221 | | | | | |
| 619 | 0.9 | 20 | AGAATCTT | csi- | | | | |
| | | | GATGATGC | miR172 | | | | |
| | | | TGCA/222 | | | | | |
| 620 | 0.9 | 20 | AGAATCTT | csi- | | | | |
| | | | GATGATGC | miR172a | | | | |
| | | | TGCA/223 | | | | | |
| 621 | 0.86 | 21 | AGAATCTT | csi- | | | | |
| | | | GATGATGC | miR172b | | | | |
| | | | GGCAA/224 | | | | | |
| 622 | 0.95 | 22 | TGGAATCTT | csi- | | | | |
| | | | GATGATGC | miR172c | | | | |
| | | | TGCAG/225 | | | | | |
| 623 | 0.86 | 21 | AGAATCCT | ghr- | | | | |
| | | | GATGATGC | miR172 | | | | |
| | | | TGCAG/226 | | | | | |
| 624 | 0.95 | 21 | AGAATCTT | gma- | | | | |
| | | | GATGATGC | miR172a | | | | |
| | | | TGCAT/227 | | | | | |
| 625 | 0.95 | 21 | AGAATCTT | gma- | | | | |
| | | | GATGATGC | miR172b | | | | |
| | | | TGCAT/228 | | | | | |
| 626 | 0.95 | 21 | GGAATCTT | gma- | | | | |
| | | | GATGATGC | miR172c | | | | |
| | | | TGCAG/229 | | | | | |
| 627 | 0.95 | 24 | GGAATCTT | gma- | | | | |
| | | | GATGATGC | miR172d | | | | |
| | | | TGCAGCAG/ | | | | | |
| | | | 230 | | | | | |
| 628 | 0.95 | 24 | GGAATCTT | gma- | | | | |
| | | | GATGATGC | miR172e | | | | |
| | | | TGCAGCAG/ | | | | | |
| | | | 231 | | | | | |
| 629 | 0.9 | 20 | AGAATCTT | gma- | | | | |
| | | | GATGATGC | miR172f | | | | |
| | | | TGCA/232 | | | | | |
| 630 | 0.95 | 21 | AGAATCTT | gra- | | | | |
| | | | GATGATGC | miR172a | | | | |
| | | | TGCAT/233 | | | | | |
| 631 | 0.9 | 21 | AAAATCTT | gra- | | | | |
| | | | GATGATGC | miR172b | | | | |
| | | | TGCAT/234 | | | | | |
| 632 | 0.86 | 21 | AGAATCCT | hvv- | | | | |
| | | | GATGATGC | miR172a | | | | |
| | | | TGCAG/235 | | | | | |
| 633 | 0.86 | 21 | AGAATCCT | hvv- | | | | |
| | | | GATGATGC | miR172b | | | | |
| | | | TGCAG/236 | | | | | |
| 634 | 0.86 | 21 | AGAATCCT | hvv- | | | | |
| | | | GATGATGC | miR172c | | | | |
| | | | TGCAG/237 | | | | | |
| 635 | 0.86 | 21 | AGAATCCT | hvv- | | | | |
| | | | GATGATGC | miR172d | | | | |
| | | | TGCAG/238 | | | | | |
| 636 | 0.95 | 21 | AGAATCTT | mes- | | | | |
| | | | GATGATGC | miR172 | | | | |
| | | | TGCAT/239 | | | | | |
| 637 | 0.86 | 21 | AGAATCCT | mtr- | | | | |
| | | | GATGATGC | miR172 | | | | |
| | | | TGCAG/240 | | | | | |
| 638 | 0.9 | 21 | GGAATCTT | mtr- | | | | |
| | | | GATGATTCT | miR172a | | | | |
| | | | GCAC/241 | | | | | |
| 639 | 0.95 | 21 | AGAATCTT | osa- | | | | |
| | | | GATGATGC | miR172a | | | | |
| | | | TGCAT/242 | | | | | |
| 640 | 1 | 21 | GGAATCTT | osa- | | | | |
| | | | GATGATGC | miR172b | | | | |
| | | | TGCAT/243 | | | | | |
| 641 | 0.9 | 21 | TGAATCTTG | osa- | | | | |
| | | | ATGATGCT | miR172c | | | | |
| | | | GCAC/244 | | | | | |
| 642 | 0.95 | 21 | AGAATCTT | osa- | | | | |
| | | | GATGATGC | miR172d | | | | |
| | | | TGCAT/245 | | | | | |
| 643 | 0.86 | 21 | AGAATCCT | osa- | | | | |
| | | | GATGATGC | miR172m | | | | |
| | | | TGCAG/246 | | | | | |
| 644 | 0.86 | 21 | AGAATCCT | osa- | | | | |
| | | | GATGATGC | miR172n | | | | |
| | | | TGCAG/247 | | | | | |
| 645 | 0.86 | 21 | AGAATCCT | osa- | | | | |
| | | | GATGATGC | miR172o | | | | |
| | | | TGCAG/248 | | | | | |
| 646 | 0.86 | 21 | AGAATCCT | osa- | | | | |
| | | | GATGATGC | miR172p | | | | |
| | | | TGCAG/249 | | | | | |
| 647 | 0.86 | 21 | AGAATCCT | pga- | | | | |
| | | | GATGATGC | miR172 | | | | |
| | | | TGCAC/250 | | | | | |
| 648 | 0.95 | 21 | AGAATCTT | ppd- | | | | |
| | | | GATGATGC | miR172a | | | | |
| | | | TGCAT/251 | | | | | |
| 649 | 0.86 | 21 | TGAATCTTG | ppd- | | | | |
| | | | ATGATGCT | miR172b | | | | |
| | | | CCAC/252 | | | | | |
| 650 | 0.86 | 21 | AGAATCCT | psi- | | | | |
| | | | GATGATGC | miR172 | | | | |
| | | | TGCAC/253 | | | | | |
| 651 | 0.95 | 21 | AGAATCTT | ptc- | | | | |
| | | | GATGATGC | miR172a | | | | |
| | | | TGCAT/254 | | | | | |
| 652 | 0.95 | 21 | AGAATCTT | ptc- | | | | |
| | | | GATGATGC | miR172b | | | | |
| | | | TGCAT/255 | | | | | |
| 653 | 0.95 | 21 | AGAATCTT | ptc- | | | | |
| | | | GATGATGC | miR172c | | | | |
| | | | TGCAT/256 | | | | | |
| 654 | 1 | 21 | GGAATCTT | ptc- | | | | |
| | | | GATGATGC | miR172d | | | | |
| | | | TGCAT/257 | | | | | |
| 655 | 1 | 21 | GGAATCTT | ptc- | | | | |
| | | | GATGATGC | miR172e | | | | |
| | | | TGCAT/258 | | | | | |
| 656 | 0.95 | 21 | AGAATCTT | ptc- | | | | |
| | | | GATGATGC | miR172f | | | | |
| | | | TGCAT/259 | | | | | |
| 657 | 0.95 | 21 | GGAATCTT | ptc- | | | | |
| | | | GATGATGC | miR172g | | | | |
| | | | TGCAG/260 | | | | | |
| 658 | 0.95 | 21 | GGAATCTT | ptc- | | | | |
| | | | GATGATGC | miR172h | | | | |
| | | | TGCAG/261 | | | | | |
| 659 | 0.86 | 21 | AGAATCCT | ptc- | | | | |
| | | | GATGATGC | miR172i | | | | |
| | | | TGCAA/262 | | | | | |
| 660 | 0.95 | 21 | GGAATCTT | rco- | | | | |
| | | | GATGATGC | miR172 | | | | |
| | | | TGCAG/263 | | | | | |
| 661 | 0.9 | 20 | AGAATCTT | sbi- | | | | |
| | | | GATGATGC | miR172a | | | | |
| | | | TGCA/264 | | | | | |
| 662 | 0.95 | 20 | GGAATCTT | sbi- | | | | |
| | | | GATGATGC | miR172b | | | | |
| | | | TGCA/265 | | | | | |
| 663 | 0.9 | 20 | AGAATCTT | sbi- | | | | |
| | | | GATGATGC | miR172c | | | | |
| | | | TGCA/266 | | | | | |
| 664/847 | 0.9 | 20 | AGAATCTT | sbi- | | | | |
| | | | GATGATGC | miR172d | | | | |
| | | | TGCA/267 | | | | | |
| 665 | 0.9 | 21 | TGAATCTTG | sbi- | | | | |
| | | | ATGATGCT | miR172e | | | | |
| | | | GCAC/268 | | | | | |
| 666 | 0.86 | 21 | AGAATCCT | sbi- | | | | |
| | | | GATGATGC | miR172f | | | | |
| | | | TGCAC/269 | | | | | |
| 667 | 0.95 | 21 | AGAATCTT | sly- | | | | |
| | | | GATGATGC | miR172a | | | | |
| | | | TGCAT/270 | | | | | |
| 668 | 0.95 | 21 | AGAATCTT | sly- | | | | |
| | | | GATGATGC | miR172b | | | | |
| | | | TGCAT/271 | | | | | |
| 669 | 0.86 | 21 | AGAATCCT | sof- | | | | |
| | | | GATGATGC | miR172a | | | | |
| | | | TGCAG/272 | | | | | |
| 670 | 0.95 | 21 | AGAATCTT | stu- | | | | |
| | | | GATGATGC | miR172 | | | | |
| | | | TGCAT/273 | | | | | |
| 671 | 0.86 | 21 | AGAATCCT | tae- | | | | |
| | | | GATGATGC | miR172a | | | | |
| | | | TGCAG/274 | | | | | |
| 672 | 0.86 | 21 | AGAATCCT | tae- | | | | |
| | | | GATGATGC | miR172b | | | | |
| | | | TGCAG/275 | | | | | |
| 673 | 0.86 | 21 | AGGATCTT | tae- | | | | |
| | | | GATGATGC | miR172c | | | | |
| | | | TGCAG/276 | | | | | |
| 674 | 0.86 | 21 | AGAATCCT | tca- | | | | |
| | | | GATGATGC | miR172 | | | | |
| | | | TGCAG/277 | | | | | |
| 675 | 0.95 | 20 | GGAATCTT | tcc- | | | | |
| | | | GATGATGC | miR172a | | | | |
| | | | TGCA/278 | | | | | |
| 676 | 0.95 | 21 | AGAATCTT | tcc- | | | | |
| | | | GATGATGC | miR172b | | | | |
| | | | TGCAT/279 | | | | | |
| 677 | 1 | 21 | GGAATCTT | tcc- | | | | |
| | | | GATGATGC | miR172c | | | | |
| | | | TGCAT/280 | | | | | |
| 678 | 0.9 | 21 | AGAATCCT | tcc- | | | | |
| | | | GATGATGC | miR172d | | | | |
| | | | TGCAT/281 | | | | | |
| 679 | 0.95 | 21 | AGAATCTT | tcc- | | | | |
| | | | GATGATGC | miR172e | | | | |
| | | | TGCAT/282 | | | | | |
| 680 | 0.9 | 21 | TGAATCTTG | vvi- | | | | |
| | | | ATGATGCT | miR172a | | | | |
| | | | ACAT/283 | | | | | |
| 681 | 0.86 | 21 | TGAATCTTG | vvi- | | | | |
| | | | ATGATGCT | miR172b | | | | |
| | | | ACAC/284 | | | | | |
| 682 | 0.95 | 21 | GGAATCTT | vvi- | | | | |
| | | | GATGATGC | miR172c | | | | |
| | | | TGCAG/285 | | | | | |
| 683 | 0.95 | 23/21 | TGAGAATC | vvi- | | | | |
| | | | TTGATGAT | miR172d | | | | |
| | | | GCTGCAT/286/ | | | | | |
| | | | AGAATCT | | | | | |
| | | | TGATGATG | | | | | |
| | | | CTGCAT/450 | | | | | |
| 684/848 | 0.9 | 20 | AGAATCTT | zma- | | | | |
| | | | GATGATGC | miR172a | | | | |
| | | | TGCA/287 | | | | | |
| 685 | 0.9 | 20 | AGAATCTT | zma- | | | | |
| | | | GATGATGC | miR172b | | | | |
| | | | TGCA/288 | | | | | |
| 686 | 0.9 | 20 | AGAATCTT | zma- | | | | |
| | | | GATGATGC | miR172c | | | | |
| | | | TGCA/289 | | | | | |
| 687 | 0.9 | 20 | AGAATCTT | zma- | | | | |
| | | | GATGATGC | miR172d | | | | |
| | | | TGCA/290 | | | | | |
| 688 | 1 | 21 | GGAATCTT | zma- | | | | |
| | | | GATGATGC | miR172f | | | | |
| | | | TGCAT/291 | | | | | |
| 689 | 0.86 | 21 | AGAATCCT | zma- | | | | |
| | | | GATGATGC | miR172m | | | | |
| | | | TGCAG/292 | | | | | |
| 690 | 0.9 | 21 | AGAATCCT | zma- | | | | |
| | | | GATGATGC | miR172n | | | | |
| | | | TGCAT/293 | | | | | |
| 691 | 0.9 | 21 | CTGAAGTG | aly- | 55 | 21 | GTGAAG | zma- |
| | | | TTTGGGGG | miR395b | | | TGTTTGG | miR395b |
| | | | GACTC/294 | | | | GGGAAC | |
| | | | | | | | TC/4 | |
| 692 | 0.86 | 21 | CTGAAGTG | aly- | | | | |
| | | | TTTGGGGG | miR395c | | | | |
| | | | GACTT/295 | | | | | |
| 693 | 0.95 | 21 | CTGAAGTG | aly- | | | | |
| | | | TTTGGGGG | miR395d | | | | |
| | | | AACTC/296 | | | | | |
| 694 | 0.95 | 21 | CTGAAGTG | aly- | | | | |
| | | | TTTGGGGG | miR395e | | | | |
| | | | AACTC/297 | | | | | |
| 695 | 0.9 | 21 | CTGAAGTG | aly- | | | | |
| | | | TTTGGGGG | miR395f | | | | |
| | | | GACTC/298 | | | | | |
| 696 | 0.95 | 21 | CTGAAGTG | aly- | | | | |
| | | | TTTGGGGG | miR395g | | | | |
| | | | AACTC/299 | | | | | |
| 697 | 0.9 | 21 | CTGAAGTG | aly- | | | | |
| | | | TTTGGGGG | miR395h | | | | |
| | | | GACTC/300 | | | | | |
| 698 | 0.9 | 21 | CTGAAGTG | aly- | | | | |
| | | | TTTGGAGG | miR395i | | | | |
| | | | AACTC/301 | | | | | |
| 699 | 0.86 | 21 | CTGAAGGG | aqc- | | | | |
| | | | TTTGGAGG | miR395a | | | | |
| | | | AACTC/302 | | | | | |
| 700 | 0.86 | 21 | CTGAAGGG | aqc- | | | | |
| | | | TTTGGAGG | miR395b | | | | |
| | | | AACTC/303 | | | | | |
| 701 | 0.95 | 21 | CTGAAGTG | ath- | | | | |
| | | | TTTGGGGG | miR395a | | | | |
| | | | AACTC/304 | | | | | |
| 702 | 0.9 | 21 | CTGAAGTG | ath- | | | | |
| | | | TTTGGGGG | miR395b | | | | |
| | | | GACTC/305 | | | | | |
| 703 | 0.9 | 21 | CTGAAGTG | ath- | | | | |
| | | | TTTGGGGG | miR395c | | | | |
| | | | GACTC/306 | | | | | |
| 704 | 0.95 | 21 | CTGAAGTG | ath- | | | | |
| | | | TTTGGGGG | miR395d | | | | |
| | | | AACTC/307 | | | | | |
| 705 | 0.95 | 21 | CTGAAGTG | ath- | | | | |
| | | | TTTGGGGG | miR395e | | | | |
| | | | AACTC/308 | | | | | |
| 706 | 0.9 | 21 | CTGAAGTG | ath- | | | | |
| | | | TTTGGGGG | miR395f | | | | |
| | | | GACTC/309 | | | | | |
| 707 | 0.95 | 20 | TGAAGTGT | bdi- | | | | |
| | | | TTGGGGGA | miR395a | | | | |
| | | | ACTC/310 | | | | | |
| 708 | 0.95 | 20 | TGAAGTGT | bdi- | | | | |
| | | | TTGGGGGA | miR395b | | | | |
| | | | ACTC/311 | | | | | |
| 709 | 0.95 | 20 | TGAAGTGT | bdi- | | | | |
| | | | TTGGGGGA | miR395c | | | | |
| | | | ACTC/312 | | | | | |
| 710 | 0.81 | 21 | AAGTGTTT | bdi- | | | | |
| | | | GGGGAACT | miR395d | | | | |
| | | | CTAGG/313 | | | | | |
| 711 | 0.95 | 20 | TGAAGTGT | bdi- | | | | |
| | | | TTGGGGGA | miR395e | | | | |
| | | | ACTC/314 | | | | | |
| 712 | 0.95 | 20 | TGAAGTGT | bdi- | | | | |
| | | | TTGGGGGA | miR395f | | | | |
| | | | ACTC/315 | | | | | |
| 713 | 0.95 | 20 | TGAAGTGT | bdi- | | | | |
| | | | TTGGGGGA | miR395g | | | | |
| | | | ACTC/316 | | | | | |
| 714 | 0.95 | 20 | TGAAGTGT | bdi- | | | | |
| | | | TTGGGGGA | miR395h | | | | |
| | | | ACTC/317 | | | | | |
| 715 | 0.95 | 20 | TGAAGTGT | bdi- | | | | |
| | | | TTGGGGGA | miR395i | | | | |
| | | | ACTC/318 | | | | | |
| 716 | 0.95 | 20 | TGAAGTGT | bdi- | | | | |
| | | | TTGGGGGA | miR395j | | | | |
| | | | ACTC/319 | | | | | |
| 717 | 0.95 | 20 | TGAAGTGT | bdi- | | | | |
| | | | TTGGGGGA | miR395k | | | | |
| | | | ACTC/320 | | | | | |
| 718 | 0.95 | 20 | TGAAGTGT | bdi- | | | | |
| | | | TTGGGGGA | miR395l | | | | |
| | | | ACTC/321 | | | | | |
| 719 | 0.95 | 20 | TGAAGTGT | bdi- | | | | |
| | | | TTGGGGGA | miR395m | | | | |
| | | | ACTC/322 | | | | | |
| 720 | 0.95 | 20 | TGAAGTGT | bdi- | | | | |
| | | | TTGGGGGA | miR395n | | | | |
| | | | ACTC/323 | | | | | |
| 721 | 0.95 | 21 | CTGAAGTG | csi- | | | | |
| | | | TTTGGGGG | miR395 | | | | |
| | | | AACTC/324 | | | | | |
| 722 | 0.9 | 21 | TTGAAGTG | ghr- | | | | |
| | | | TTTGGGGG | miR395a | | | | |
| | | | AACTT/325 | | | | | |
| 723 | 0.86 | 21 | CTAAAGTG | ghr- | | | | |
| | | | TTTAGGGG | miR395c | | | | |
| | | | AACTC/326 | | | | | |
| 724 | 0.95 | 21 | CTGAAGTG | ghr- | | | | |
| | | | TTTGGGGG | miR395d | | | | |
| | | | AACTC/327 | | | | | |
| 725 | 0.95 | 21 | ATGAAGTG | gma- | | | | |
| | | | TTTGGGGG | miR395 | | | | |
| | | | AACTC/328 | | | | | |
| 726 | 0.95 | 21 | ATGAAGTG | mtr- | | | | |
| | | | TTTGGGGG | miR395a | | | | |
| | | | AACTC/329 | | | | | |
| 727 | 0.9 | 21 | ATGAAGTA | mtr- | | | | |
| | | | TTTGGGGG | miR395b | | | | |
| | | | AACTC/330 | | | | | |
| 728 | 0.95 | 21 | ATGAAGTG | mtr- | | | | |
| | | | TTTGGGGG | miR395c | | | | |
| | | | AACTC/331 | | | | | |
| 729 | 0.95 | 21 | ATGAAGTG | mtr- | | | | |
| | | | TTTGGGGG | miR395d | | | | |
| | | | AACTC/332 | | | | | |
| 730 | 0.95 | 21 | ATGAAGTG | mtr- | | | | |
| | | | TTTGGGGG | miR395e | | | | |
| | | | AACTC/333 | | | | | |
| 731 | 0.95 | 21 | ATGAAGTG | mtr- | | | | |
| | | | TTTGGGGG | miR395f | | | | |
| | | | AACTC/334 | | | | | |
| 732 | 0.95 | 21 | TTGAAGTG | mtr- | | | | |
| | | | TTTGGGGG | miR395g | | | | |
| | | | AACTC/335 | | | | | |
| 733 | 0.9 | 21 | ATGAAGTG | mtr- | | | | |
| | | | TTTGGGGG | miR395h | | | | |
| | | | AACTT/336 | | | | | |
| 734 | 0.95 | 21 | ATGAAGTG | mtr- | | | | |
| | | | TTTGGGGG | miR395i | | | | |
| | | | AACTC/337 | | | | | |
| 735 | 0.95 | 21 | ATGAAGTG | mtr- | | | | |
| | | | TTTGGGGG | miR395j | | | | |
| | | | AACTC/338 | | | | | |
| 736 | 0.95 | 21 | ATGAAGTG | mtr- | | | | |
| | | | TTTGGGGG | miR395k | | | | |
| | | | AACTC/339 | | | | | |
| 737 | 0.95 | 21 | ATGAAGTG | mtr- | | | | |
| | | | TTTGGGGG | miR395l | | | | |
| | | | AACTC/340 | | | | | |
| 738 | 0.95 | 21 | ATGAAGTG | mtr- | | | | |
| | | | TTTGGGGG | miR395m | | | | |
| | | | AACTC/341 | | | | | |
| 739 | 0.95 | 21 | ATGAAGTG | mtr- | | | | |
| | | | TTTGGGGG | miR395n | | | | |
| | | | AACTC/342 | | | | | |
| 740 | 0.95 | 21 | ATGAAGTG | mtr- | | | | |
| | | | TTTGGGGG | miR395o | | | | |
| | | | AACTC/343 | | | | | |
| 741 | 0.9 | 21 | TTGAAGCG | mtr- | | | | |
| | | | TTTGGGGG | miR395p | | | | |
| | | | AACTC/344 | | | | | |
| 742 | 0.95 | 21 | ATGAAGTG | mtr- | | | | |
| | | | TTTGGGGG | miR395q | | | | |
| | | | AACTC/345 | | | | | |
| 743 | 0.95 | 21 | ATGAAGTG | mtr- | | | | |
| | | | TTTGGGGG | miR395r | | | | |
| | | | AACTC/346 | | | | | |
| 744 | 0.95 | 21 | GTGAAGTG | osa- | | | | |
| | | | CTTGGGGG | miR395a | | | | |
| | | | AACTC/347 | | | | | |
| 745 | 0.9 | 20 | TGAAGTGC | osa- | | | | |
| | | | TTGGGGGA | miR395a.2 | | | | |
| | | | ACTC/348 | | | | | |
| 746 | 1 | 21 | GTGAAGTG | osa- | | | | |
| | | | TTTGGGGG | miR395b | | | | |
| | | | AACTC/349 | | | | | |
| 747 | 0.95 | 21 | GTGAAGTG | osa- | | | | |
| | | | TTTGGAGG | miR395c | | | | |
| | | | AACTC/350 | | | | | |
| 748 | 1 | 21 | GTGAAGTG | osa- | | | | |
| | | | TTTGGGGG | miR395d | | | | |
| | | | AACTC/351 | | | | | |
| 749 | 1 | 21 | GTGAAGTG | osa- | | | | |
| | | | TTTGGGGG | miR395e | | | | |
| | | | AACTC/352 | | | | | |
| 750 | 0.95 | 21 | GTGAATTG | osa- | | | | |
| | | | TTTGGGGG | miR395f | | | | |
| | | | AACTC/353 | | | | | |
| 751 | 1 | 21 | GTGAAGTG | osa- | | | | |
| | | | TTTGGGGG | miR395g | | | | |
| | | | AACTC/354 | | | | | |
| 752 | 1 | 21 | GTGAAGTG | osa- | | | | |
| | | | TTTGGGGG | miR395h | | | | |
| | | | AACTC/355 | | | | | |
| 753 | 1 | 21 | GTGAAGTG | osa- | | | | |
| | | | TTTGGGGG | miR395i | | | | |
| | | | AACTC/356 | | | | | |
| 754 | 1 | 21 | GTGAAGTG | osa- | | | | |
| | | | TTTGGGGG | miR395j | | | | |
| | | | AACTC/357 | | | | | |
| 755 | 1 | 21 | GTGAAGTG | osa- | | | | |
| | | | TTTGGGGG | miR395k | | | | |
| | | | AACTC/358 | | | | | |
| 756 | 1 | 21 | GTGAAGTG | osa- | | | | |
| | | | TTTGGGGG | miR395l | | | | |
| | | | AACTC/359 | | | | | |
| 757 | 1 | 21 | GTGAAGTG | osa- | | | | |
| | | | TTTGGGGG | miR395m | | | | |
| | | | AACTC/360 | | | | | |
| 758 | 1 | 21 | GTGAAGTG | osa- | | | | |
| | | | TTTGGGGG | miR395n | | | | |
| | | | AACTC/361 | | | | | |
| 759 | 0.9 | 21 | ATGAAGTG | osa- | | | | |
| | | | TTTGGAGG | miR395o | | | | |
| | | | AACTC/362 | | | | | |
| 760 | 1 | 21 | GTGAAGTG | osa- | | | | |
| | | | TTTGGGGG | miR395p | | | | |
| | | | AACTC/363 | | | | | |
| 761 | 1 | 21 | GTGAAGTG | osa- | | | | |
| | | | TTTGGGGG | miR395q | | | | |
| | | | AACTC/364 | | | | | |
| 762 | 1 | 21 | GTGAAGTG | osa- | | | | |
| | | | TTTGGGGG | miR395r | | | | |
| | | | AACTC/365 | | | | | |
| 763 | 1 | 21 | GTGAAGTG | osa- | | | | |
| | | | TTTGGGGG | miR395s | | | | |
| | | | AACTC/366 | | | | | |
| 764 | 0.95 | 21 | GTGAAGTG | osa- | | | | |
| | | | TTTGGGGA | miR395t | | | | |
| | | | AACTC/367 | | | | | |
| 765 | 0.9 | 21 | GTGAAGCG | osa- | | | | |
| | | | TTTGGGGG | miR395u | | | | |
| | | | AAATC/368 | | | | | |
| 766 | 0.9 | 21 | GTGAAGTA | osa- | | | | |
| | | | TTTGGCGG | miR395v | | | | |
| | | | AACTC/369 | | | | | |
| 767 | 0.81 | 22 | GTGAAGTG | osa- | | | | |
| | | | TTTGGGGG | miR395w | | | | |
| | | | ATTCTC/370 | | | | | |
| 768 | 0.86 | 21 | GTGAAGTG | osa- | | | | |
| | | | TTTGGAGT | miR395x | | | | |
| | | | AGCTC/371 | | | | | |
| 769 | 1 | 21 | GTGAAGTG | osa- | | | | |
| | | | TTTGGGGG | miR395y | | | | |
| | | | AACTC/372 | | | | | |
| 770 | 0.86 | 21 | CTGAAGTG | pab- | | | | |
| | | | TTTGGAGG | miR395 | | | | |
| | | | AACTT/373 | | | | | |
| 771 | 0.86 | 21 | CTGAAGGG | ptc- | | | | |
| | | | TTTGGAGG | miR395a | | | | |
| | | | AACTC/374 | | | | | |
| 772 | 0.95 | 21 | CTGAAGTG | ptc- | | | | |
| | | | TTTGGGGG | miR395b | | | | |
| | | | AACTC/375 | | | | | |
| 773 | 0.95 | 21 | CTGAAGTG | ptc- | | | | |
| | | | TTTGGGGG | miR395c | | | | |
| | | | AACTC/376 | | | | | |
| 774 | 0.95 | 21 | CTGAAGTG | ptc- | | | | |
| | | | TTTGGGGG | miR395d | | | | |
| | | | AACTC/377 | | | | | |
| 775 | 0.95 | 21 | CTGAAGTG | ptc- | | | | |
| | | | TTTGGGGG | miR395e | | | | |
| | | | AACTC/378 | | | | | |
| 776 | 0.95 | 21 | CTGAAGTG | ptc- | | | | |
| | | | TTTGGGGG | miR395f | | | | |
| | | | AACTC/379 | | | | | |
| 777 | 0.95 | 21 | CTGAAGTG | ptc- | | | | |
| | | | TTTGGGGG | miR395g | | | | |
| | | | AACTC/380 | | | | | |
| 778 | 0.95 | 21 | CTGAAGTG | ptc- | | | | |
| | | | TTTGGGGG | miR395h | | | | |
| | | | AACTC/381 | | | | | |
| 779 | 0.95 | 21 | CTGAAGTG | ptc- | | | | |
| | | | TTTGGGGG | miR395i | | | | |
| | | | AACTC/382 | | | | | |
| 780 | 0.95 | 21 | CTGAAGTG | ptc- | | | | |
| | | | TTTGGGGG | miR395j | | | | |
| | | | AACTC/383 | | | | | |
| 781 | 0.95 | 21 | CTGAAGTG | rco- | | | | |
| | | | TTTGGGGG | miR395a | | | | |
| | | | AACTC/384 | | | | | |
| 782 | 0.95 | 21 | CTGAAGTG | rco- | | | | |
| | | | TTTGGGGG | miR395b | | | | |
| | | | AACTC/385 | | | | | |
| 783 | 0.95 | 21 | CTGAAGTG | rco- | | | | |
| | | | TTTGGGGG | miR395c | | | | |
| | | | AACTC/386 | | | | | |
| 784 | 0.95 | 21 | CTGAAGTG | rco- | | | | |
| | | | TTTGGGGG | miR395d | | | | |
| | | | AACTC/387 | | | | | |
| 785 | 0.95 | 21 | CTGAAGTG | rco- | | | | |
| | | | TTTGGGGG | miR395e | | | | |
| | | | AACTC/388 | | | | | |
| 786 | 1 | 21 | GTGAAGTG | sbi- | | | | |
| | | | TTTGGGGG | miR395a | | | | |
| | | | AACTC/389 | | | | | |
| 787 | 1 | 21 | GTGAAGTG | sbi- | | | | |
| | | | TTTGGGGG | miR395b | | | | |
| | | | AACTC/390 | | | | | |
| 788/849 | 1 | 21 | GTGAAGTG | sbi- | | | | |
| | | | TTTGGGGG | miR395c | | | | |
| | | | AACTC/391 | | | | | |
| 789/850 | 1 | 21 | GTGAAGTG | sbi- | | | | |
| | | | TTTGGGGG | miR395d | | | | |
| | | | AACTC/392 | | | | | |
| 790 | 1 | 21 | GTGAAGTG | sbi- | | | | |
| | | | TTTGGGGG | miR395e | | | | |
| | | | AACTC/393 | | | | | |
| 791 | 0.95 | 21 | ATGAAGTG | sbi- | | | | |
| | | | TTTGGGGG | miR395f | | | | |
| | | | AACTC/394 | | | | | |
| 792 | 1 | 21 | GTGAAGTG | sbi- | | | | |
| | | | TTTGGGGG | miR395g | | | | |
| | | | AACTC/395 | | | | | |
| 793 | 1 | 21 | GTGAAGTG | sbi- | | | | |
| | | | TTTGGGGG | miR395h | | | | |
| | | | AACTC/396 | | | | | |
| 794 | 1 | 21 | GTGAAGTG | sbi- | | | | |
| | | | TTTGGGGG | miR395i | | | | |
| | | | AACTC/397 | | | | | |
| 795 | 1 | 21 | GTGAAGTG | sbi- | | | | |
| | | | TTTGGGGG | miR395j | | | | |
| | | | AACTC/398 | | | | | |
| 796 | 0.95 | 21 | GTGAAGTG | sbi- | | | | |
| | | | TTTGGAGG | miR395k | | | | |
| | | | AACTC/399 | | | | | |
| 797 | 0.95 | 21 | GTGAAGTG | sbi- | | | | |
| | | | CTTGGGGG | miR395l | | | | |
| | | | AACTC/400 | | | | | |
| 798 | 0.95 | 21 | CTGAAGTG | sde- | | | | |
| | | | TTTGGGGG | miR395 | | | | |
| | | | AACTC/401 | | | | | |
| 799 | 0.95 | 22 | CTGAAGTG | sly- | | | | |
| | | | TTTGGGGG | miR395a | | | | |
| | | | AACTCC/402 | | | | | |
| 800 | 0.95 | 22 | CTGAAGTG | sly- | | | | |
| | | | TTTGGGGG | miR395b | | | | |
| | | | AACTCC/403 | | | | | |
| 801 | 1 | 21 | GTGAAGTG | tae- | | | | |
| | | | TTTGGGGG | miR395a | | | | |
| | | | AACTC/404 | | | | | |
| 802 | 0.95 | 20 | TGAAGTGT | tae- | | | | |
| | | | TTGGGGGA | miR395b | | | | |
| | | | ACTC/405 | | | | | |
| 803 | 0.95 | 21 | CTGAAGTG | tcc- | | | | |
| | | | TTTGGGGG | miR395a | | | | |
| | | | AACTC/406 | | | | | |
| 804 | 0.95 | 21 | CTGAAGTG | tcc- | | | | |
| | | | TTTGGGGG | miR395b | | | | |
| | | | AACTC/407 | | | | | |
| 805 | 0.95 | 21 | CTGAAGTG | vvi- | | | | |
| | | | TTTGGGGG | miR395a | | | | |
| | | | AACTC/408 | | | | | |
| 806 | 0.95 | 21 | CTGAAGTG | vvi- | | | | |
| | | | TTTGGGGG | miR395b | | | | |
| | | | AACTC/409 | | | | | |
| 807 | 0.95 | 21 | CTGAAGTG | vvi- | | | | |
| | | | TTTGGGGG | miR395c | | | | |
| | | | AACTC/410 | | | | | |
| 808 | 0.95 | 21 | CTGAAGTG | vvi- | | | | |
| | | | TTTGGGGG | miR395d | | | | |
| | | | AACTC/411 | | | | | |
| 809 | 0.95 | 21 | CTGAAGTG | vvi- | | | | |
| | | | TTTGGGGG | miR395e | | | | |
| | | | AACTC/412 | | | | | |
| 810 | 0.95 | 21 | CTGAAGTG | vvi- | | | | |
| | | | TTTGGGGG | miR395f | | | | |
| | | | AACTC/413 | | | | | |
| 811 | 0.95 | 21 | CTGAAGTG | vvi- | | | | |
| | | | TTTGGGGG | miR395g | | | | |
| | | | AACTC/414 | | | | | |
| 812 | 0.95 | 21 | CTGAAGTG | vvi- | | | | |
| | | | TTTGGGGG | miR395h | | | | |
| | | | AACTC/415 | | | | | |
| 813 | 0.95 | 21 | CTGAAGTG | vvi- | | | | |
| | | | TTTGGGGG | miR395i | | | | |
| | | | AACTC/416 | | | | | |
| 814 | 0.95 | 21 | CTGAAGTG | vvi- | | | | |
| | | | TTTGGGGG | miR395j | | | | |
| | | | AACTC/417 | | | | | |
| 815 | 0.95 | 21 | CTGAAGTG | vvi- | | | | |
| | | | TTTGGGGG | miR395k | | | | |
| | | | AACTC/418 | | | | | |
| 816 | 0.95 | 21 | CTGAAGTG | vvi- | | | | |
| | | | TTTGGGGG | miR395l | | | | |
| | | | AACTC/419 | | | | | |
| 817 | 0.95 | 21 | CTGAAGTG | vvi- | | | | |
| | | | TTTGGGGG | miR395m | | | | |
| | | | AACTC/420 | | | | | |
| 818 | 0.81 | 21 | CTGAAGAG | vvi- | | | | |
| | | | TCTGGAGG | miR395n | | | | |
| | | | AACTC/421 | | | | | |
| 819 | 1 | 21 | GTGAAGTG | zma- | | | | |
| | | | TTTGGGGG | miR395a | | | | |
| | | | AACTC/422 | | | | | |
| 820 | 0.95 | 21 | GTGAAGTG | zma- | | | | |
| | | | TTTGGAGG | miR395c | | | | |
| | | | AACTC/423 | | | | | |
| 821/851 | 1.00/0.90 | 21/20 | GTGAAGTG | zma- | | | | |
| | | | TTTGGGGG | miR395d | | | | |
| | | | AACTC/424/ | | | | | |
| | | | GTGAAGTG | | | | | |
| | | | TTTGGAGG | | | | | |
| | | | AACT/451 | | | | | |
| 822/852 | 1.00/0.95 | 21 | GTGAAGTG | zma- | | | | |
| | | | TTTGGGGG | miR395e | | | | |
| | | | AACTC/425/ | | | | | |
| | | | GTGAAGTG | | | | | |
| | | | TTTGGAGG | | | | | |
| | | | AACTC/452 | | | | | |
| 823/853 | 1.00/0.90 | 21 | GTGAAGTG | zma- | | | | |
| | | | TTTGGGGG | miR395f | | | | |
| | | | AACTC/426/ | | | | | |
| | | | GTGAAGTG | | | | | |
| | | | TTTGAGGA | | | | | |
| | | | AACTC/453 | | | | | |
| 824 | 1 | 21 | GTGAAGTG | zma- | | | | |
| | | | TTTGGGGG | miR395g | | | | |
| | | | AACTC/427 | | | | | |
| 825 | 1 | 21 | GTGAAGTG | zma- | | | | |
| | | | TTTGGGGG | miR395h | | | | |
| | | | AACTC/428 | | | | | |
| 826 | 1 | 21 | GTGAAGTG | zma- | | | | |
| | | | TTTGGGGG | miR395i | | | | |
| | | | AACTC/429 | | | | | |
| 827 | 1 | 21 | GTGAAGTG | zma- | | | | |
| | | | TTTGGGGG | miR395j | | | | |
| | | | AACTC/430 | | | | | |
| 828 | 0.9 | 21 | GTGAAGTG | zma- | | | | |
| | | | TTTGAGGA | miR395k | | | | |
| | | | AACTC/431 | | | | | |
| 829 | 0.95 | 21 | GTGAAGTG | zma- | | | | |
| | | | TTTGGAGG | miR395l | | | | |
| | | | AACTC/432 | | | | | |
| 830 | 0.95 | 21 | GTGAAGTG | zma- | | | | |
| | | | TTTGGAGG | miR395m | | | | |
| | | | AACTC/433 | | | | | |
| 831 | 1 | 21 | GTGAAGTG | zma- | | | | |
| | | | TTTGGGGG | miR395n | | | | |
| | | | AACTC/434 | | | | | |
| 832 | 0.95 | 21 | GTGAAGTG | zma- | | | | |
| | | | TTTGGGTG | miR395o | | | | |
| | | | AACTC/435 | | | | | |
| 833 | 1 | 21 | GTGAAGTG | zma- | | | | |
| | | | TTTGGGGG | miR395p | | | | |
| | | | AACTC/436 | | | | | |
| 834 | 0.86 | 21 | AGAAGAGA | aqc- | 56 | 21 | AGAAGA | zma- |
| | | | GAGAGCAC | miR529 | | | GAGAGA | miR529 |
| | | | AACCC/437 | | | | GTACAG | |
| | | | | | | | CCT/1 | |
| 835 | 1 | 21 | AGAAGAGA | bdi- | | | | |
| | | | GAGAGTAC | miR529 | | | | |
| | | | AGCCT/438 | | | | | |
| 836 | 0.9 | 21 | AGAAGAGA | far- | | | | |
| | | | GAGAGCAC | miR529 | | | | |
| | | | AGCTT/439 | | | | | |
| 837 | 0.95 | 21 | AGAAGAGA | osa- | | | | |
| | | | GAGAGTAC | miR529b | | | | |
| | | | AGCTT/440 | | | | | |
| 838 | 0.86 | 21 | CGAAGAGA | ppt- | | | | |
| | | | GAGAGCAC | miR529a | | | | |
| | | | AGCCC/441 | | | | | |
| 839 | 0.86 | 21 | CGAAGAGA | ppt- | | | | |
| | | | GAGAGCAC | miR529b | | | | |
| | | | AGCCC/442 | | | | | |
| 840 | 0.86 | 21 | CGAAGAGA | ppt- | | | | |
| | | | GAGAGCAC | miR529c | | | | |
| | | | AGCCC/443 | | | | | |
| 841 | 0.9 | 21 | AGAAGAGA | ppt- | | | | |
| | | | GAGAGCAC | miR529d | | | | |
| | | | AGCCC/444 | | | | | |
| 842 | 0.95 | 21 | AGAAGAGA | ppt- | | | | |
| | | | GAGAGTAC | miR529e | | | | |
| | | | AGCCC/445 | | | | | |
| 843 | 0.95 | 21 | AGAAGAGA | ppt- | | | | |
| | | | GAGAGTAC | miR529f | | | | |
| | | | AGCCC/446 | | | | | |
| 844 | 0.81 | 21 | CGAAGAGA | ppt- | | | | |
| | | | GAGAGCAC | miR529g | | | | |
| | | | AGTCC/447 | | | | | |
| 845 | 0.9 | 22 | TAGCCAAG | bdi- | | 21 | TAGCCA | Predicted |
| | | | GATGATTT | miR169k | | | AGCATG | zma mir |
| | | | GCCTGT/448 | | | | ATTTGCC | 50601 |
| | | | | | | | CG/5 | |
| 846 | 0.9 | 21 | TAGCCAAG | sbi- | | | | |
| | | | GATGATTT | miR1690 | | | | |
| | | | GCCTG/449 | | | | | |

### Example 3

### Identification of miRNAs Associated with Increased NUE and Target Prediction Using Bioinformatics Tools

miRNAs that are associated with improved NUE and/or abiotic or biotic stress tolerance were identified by computational algorithms that analyze RNA expression profiles alongside publicly available gene and protein databases. A high throughput screening was performed on microarrays loaded with miRNAs that were found to be differentially expressed under multiple stress and optimal environmental conditions and in different plant tissues. The initial trait-associated miRNAs were later validated by quantitative Real Time PCR (qRT-PCR).

Target prediction—orthologous genes to the genes of interest in maize and/or Arabidopsis were found through a bioinformatic tool that analyzes publicly available genomic as well as expression and gene annotation databases from multiple plant species. Homologous as well as orthologous protein and nucleotide sequences of target genes of the small RNA sequences of the invention, were found using BLAST having at least 70% identity on at least 60% of the entire master (maize) gene length, and are summarized in Tables 5-6 below.

TABLE 5
Target Genes of Small RNA Molecules that are upregulated during NUE.

| | Protein | | | | |
|---|---|---|---|---|---|
| Nucleotide | Sequence | | | | Nucleotide |
| Sequence | seq id | | % | | NCBI GI |
| seq id no: | no: | Organism | Identity | Anotation | number |
| 895 | 854 | Zea mays | 1 | hypothetical | 293331460 |
| | | | | protein | |
| | | | | LOC100384547 | |
| | | | | [Zea | |
| | | | | mays] | |
| | | | | &gt; gi\|238005886\| | |
| | | | | gb\|ACR33978.1\| | |
| | | | | unknown | |
| | | | | [Zea mays] | |
| | 855 | Zea mays | 1 | putative gag- | 23928433 |
| | | | | pol | |
| | | | | polyprotein | |
| | | | | [Zea mays] | |
| 896 | 856 | Eulaliopsis | 1 | embryonic | 315493433 |
| | | binata | | flower 1 | |
| | | | | protein | |
| | | | | [Eulaliopsis | |
| | | | | binata] | |
| 897 | 857 | Zea mays | 0.923445 | EMF-like | 85062576 |
| | | | | [Zea mays] | |
| 898 | 858 | Zea mays | 0.9346093 | VEF family | 162461707 |
| | | | | protein [Zea | |
| | | | | mays] | |
| | | | | &gt; gi\|29569111\| | |
| | | | | gb\|AAO84022.1\| | |
| | | | | VEF family | |
| | | | | protein [Zea | |
| | | | | mays] | |
| | | | | &gt; gi\|60687422\| | |
| | | | | gb\|AAX35735.1\| | |
| | | | | embryonic | |
| | | | | flower 2 [Zea | |
| | | | | mays] | |
| 899 | 859 | Dendrocalamus | 0.8054226 | EMF2 | 82469918 |
| | | latiflorus | | [Dendrocalamus | |
| | | | | latiflorus] | |
| 900 | 860 | Triticum | 0.7974482 | embryonic | 62275660 |
| | | aestivum | | flower 2 | |
| | | | | [Triticum | |
| | | | | aestivum] | |
| 901 | 861 | Oryza | 0.7575758 | Os09g0306800 | 115478459 |
| | | sativa | | [Oryza | |
| | | Japonica | | sativa | |
| | | Group | | Japonica | |
| | | | | Group] | |
| | | | | &gt; gi\|255678755\| | |
| | | | | dbj\|BAF24739.2\| | |
| | | | | Os09g0306800 | |
| | | | | [Oryza | |
| | | | | sativa | |
| | | | | Japonica | |
| | | | | Group] | |
| | 862 | Oryza | 0.7575758 | putative VEF | 51091694 |
| | | sativa | | family | |
| | | Japonica | | protein | |
| | | Group | | [Oryza sativa | |
| | | | | Japonica | |
| | | | | Group] | |
| 902 | 863 | Eulaliopsis | 0.7575758 | embryonic | 315493435 |
| | | binata | | flower 2 | |
| | | | | protein | |
| | | | | [Eulaliopsis | |
| | | | | binata] | |
| 903 | 864 | Hordeum | 0.76874 | predicted | 326503299 |
| | | vulgare | | protein | |
| | | subsp. | | [Hordeum | |
| | | vulgare | | vulgare | |
| | | | | subsp. | |
| | | | | vulgare] | |
| 904 | 865 | Hordeum | 0.7703349 | HvEMF2b | 66796110 |
| | | vulgare | | [Hordeum | |
| | | | | vulgare] | |
| 905 | 866 | Zea mays | 1 | VEF family | 162461707 |
| | | | | protein [Zea | |
| | | | | mays] | |
| | | | | &gt; gi\|29569111\| | |
| | | | | gb\|AAO84022.1\| | |
| | | | | VEF family | |
| | | | | protein [Zea | |
| | | | | mays] | |
| | | | | &gt; gi\|60687422\| | |
| | | | | gb\|AAX35735.1\| | |
| | | | | embryonic | |
| | | | | flower 2 [Zea | |
| | | | | mays] | |
| 906 | 867 | Zea mays | 0.9792332 | EMF-like | 85062576 |
| | | | | [Zea mays] | |
| 907 | 868 | Eulaliopsis | 0.9361022 | embryonic | 315493433 |
| | | binata | | flower 1 | |
| | | | | protein | |
| | | | | [Eulaliopsis | |
| | | | | binata] | |
| 908 | 869 | Dendrocalamus | 0.8083067 | EMF2 | 82469918 |
| | | latiflorus | | [Dendrocalamus | |
| | | | | latiflorus] | |
| 909 | 870 | Triticum | 0.8019169 | embryonic | 62275660 |
| | | aestivum | | flower 2 | |
| | | | | [Triticum | |
| | | | | aestivum] | |
| 910 | 871 | Oryza | 0.7571885 | Os09g0306800 | 115478459 |
| | | sativa | | [Oryza | |
| | | Japonica | | sativa | |
| | | Group | | Japonica | |
| | | | | Group] | |
| | | | | &gt; gi\|255678755\| | |
| | | | | dbj\|BAF24739.2\| | |
| | | | | Os09g0306800 | |
| | | | | [Oryza | |
| | | | | sativa | |
| | | | | Japonica | |
| | | | | Group] | |
| | 872 | Oryza | 0.7555911 | putative VEF | 51091694 |
| | | sativa | | family | |
| | | Japonica | | protein | |
| | | Group | | [Oryza sativa | |
| | | | | Japonica | |
| | | | | Group] | |
| 911 | 873 | Eulaliopsis | 0.7635783 | embryonic | 315493435 |
| | | binata | | flower 2 | |
| | | | | protein | |
| | | | | [Eulaliopsis | |
| | | | | binata] | |
| 912 | 874 | Hordeum | 0.7747604 | predicted | 326503299 |
| | | vulgare | | protein | |
| | | subsp. | | [Hordeum | |
| | | vulgare | | vulgare | |
| | | | | subsp. | |
| | | | | vulgare] | |
| 913 | 875 | Hordeum | 0.7763578 | HvEMF2b | 66796110 |
| | | vulgare | | [Hordeum | |
| | | | | vulgare] | |
| | 876 | Sorghum | 1 | hypothetical | 255761094 |
| | | bicolor | | protein | |
| | | | | SORBIDRAFT_04g031920 | |
| | | | | [Sorghum | |
| | | | | bicolor] | |
| | | | | &gt; gi\|241934313\| | |
| | | | | gb\|EES07458.1\| | |
| | | | | hypothetical | |
| | | | | protein | |
| | | | | SORBIDRAFT_04g031920 | |
| | | | | [Sorghum | |
| | | | | bicolor] | |
| 914 | 877 | Zea mays | 0.9425287 | unknown | 223972968 |
| | | | | [Zea mays] | |
| 915 | 878 | Zea mays | 0.941092 | hypothetical | 308044322 |
| | | | | protein | |
| | | | | LOC100501893 | |
| | | | | [Zea | |
| | | | | mays] | |
| | | | | &gt; gi\|238011698\| | |
| | | | | gb\|ACR36884.1\| | |
| | | | | unknown | |
| | | | | [Zea mays] | |
| | 879 | Oryza | 0.8706897 | RecName: | |
| | | sativa | | Full = SPX | |
| | | Japonica | | domain- | |
| | | Group | | containing | |
| | | | | membrane | |
| | | | | protein | |
| | | | | Os02g45520 | |
| | | | | &gt; gi\|306756291\| | |
| | | | | sp\|A2X8A7.2\| | |
| | | | | SPXM1_ORYSI | |
| | | | | RecName: | |
| | | | | Full = SPX | |
| | | | | domain- | |
| | | | | containing | |
| | | | | membrane | |
| | | | | protein | |
| | | | | OsI_08463 | |
| | | | | &gt; gi\|50252990\| | |
| | | | | dbj\|BAD29241.1\| | |
| | | | | SPX | |
| | | | | (SYG1/Pho81/ | |
| | | | | XPR1) | |
| | | | | domain- | |
| | | | | containing | |
| | | | | protein-like | |
| | | | | [Oryza sativa | |
| | | | | Japonica | |
| | | | | Group] | |
| | | | | &gt; gi\|50253121\| | |
| | | | | dbj\|BAD29367.1\| | |
| | | | | SPX | |
| | | | | (SYG1/Pho81/ | |
| | | | | XPR1) | |
| | | | | domain- | |
| | | | | containing | |
| | | | | protein-like | |
| | | | | [Oryza sativa | |
| | | | | Japonica | |
| | | | | Group] | |
| 916 | 880 | Hordeum | 0.8347701 | predicted | 326502341 |
| | | vulgare | | protein | |
| | | subsp. | | [Hordeum | |
| | | vulgare | | vulgare | |
| | | | | subsp. | |
| | | | | vulgare] | |
| | 881 | Oryza | 0.808908 | OSJNBa0019K04.6 | 38605939 |
| | | sativa | | [Oryza sativa | |
| | | Japonica | | Japonica | |
| | | Group | | Group] | |
| | | | | &gt; gi\|125591348\| | |
| | | | | gb\|EAZ31698.1\| | |
| | | | | hypothetical | |
| | | | | protein | |
| | | | | OsJ_15847 | |
| | | | | [Oryza sativa | |
| | | | | Japonica | |
| | | | | Group] | |
| 917 | 882 | Oryza | 0.808908 | Os04g0573000 | 115460021 |
| | | sativa | | [Oryza | |
| | | Japonica | | sativa | |
| | | Group | | Japonica | |
| | | | | Group] | |
| | | | | &gt; gi\|306756012\| | |
| | | | | sp\|B8AT51.1\| | |
| | | | | SPXM2_ORYSI | |
| | | | | RecName: | |
| | | | | Full = SPX | |
| | | | | domain- | |
| | | | | containing | |
| | | | | membrane | |
| | | | | protein | |
| | | | | OsI_17046 | |
| | | | | &gt; gi\|306756288\| | |
| | | | | sp\|Q0JAW2.2\| | |
| | | | | SPXM2_ORYSJ | |
| | | | | RecName: | |
| | | | | Full = SPX | |
| | | | | domain- | |
| | | | | containing | |
| | | | | membrane | |
| | | | | protein | |
| | | | | Os04g0573000 | |
| | | | | &gt; gi\|215694614\| | |
| | | | | dbj\|BAG89805.1\| | |
| | | | | unnamed | |
| | | | | protein | |
| | | | | product | |
| | | | | [Oryza sativa | |
| | | | | Japonica | |
| | | | | Group] | |
| | | | | &gt; gi\|218195403\| | |
| | | | | gb\|EEC77830.1\| | |
| | | | | hypothetical | |
| | | | | protein | |
| | | | | OsI_17046 | |
| | | | | [Oryza sativa | |
| | | | | Indica | |
| | | | | Group] | |
| | | | | &gt; gi\|255675707\| | |
| | | | | dbj\|BAF15525.2\| | |
| | | | | Os04g0573000 | |
| | | | | [Oryza | |
| | | | | sativa | |
| | | | | Japonica | |
| | | | | Group] | |
| 918 | 883 | Oryza | 0.8060345 | OSIGBa0147H17.5 | 116309919 |
| | | sativa | | [Oryza sativa | |
| | | Indica | | Indica | |
| | | Group | | Group] | |
| | 884 | Sorghum | 0.7844828 | hypothetical | 255761094 |
| | | bicolor | | protein | |
| | | | | SORBIDRAFT_06g025950 | |
| | | | | [Sorghum | |
| | | | | bicolor] | |
| | | | | &gt; gi\|241938147\| | |
| | | | | gb\|EES11292.1\| | |
| | | | | hypothetical | |
| | | | | protein | |
| | | | | SORBIDRAFT_06g025950 | |
| | | | | [Sorghum | |
| | | | | bicolor] | |
| 919 | 885 | Vitis | 0.7212644 | PREDICTED: | 225426756 |
| | | vinifera | | hypothetical | |
| | | | | protein [Vitis | |
| | | | | vinifera] | |
| | | | | &gt; gi\|297742609\| | |
| | | | | emb\|CBI34758.3\| | |
| | | | | unnamed | |
| | | | | protein | |
| | | | | product | |
| | | | | [Vitis | |
| | | | | vinifera] | |
| | 886 | Sorghum | 1 | hypothetical | 255761094 |
| | | bicolor | | protein | |
| | | | | SORBIDRAFT_02g027920 | |
| | | | | [Sorghum | |
| | | | | bicolor] | |
| | | | | &gt; gi\|241925925\| | |
| | | | | gb\|EER99069.1\| | |
| | | | | hypothetical | |
| | | | | protein | |
| | | | | SORBIDRAFT_02g027920 | |
| | | | | [Sorghum | |
| | | | | bicolor] | |
| 920 | 887 | Zea mays | 0.8819188 | hypothetical | 226498793 |
| | | | | protein | |
| | | | | LOC100279277 | |
| | | | | [Zea | |
| | | | | mays] | |
| | | | | &gt; gi\|219884365\| | |
| | | | | gb\|ACL52557.1\| | |
| | | | | unknown | |
| | | | | [Zea mays] | |
| 921 | 888 | Zea mays | 0.8523985 | unknown | 224030802 |
| | | | | [Zea mays] | |
| 922 | 889 | Zea mays | 0.8523985 | hypothetical | 226530255 |
| | | | | protein | |
| | | | | LOC100278416 | |
| | | | | [Zea | |
| | | | | mays] | |
| | | | | &gt; gi\|195652339\| | |
| | | | | gb\|ACG45637.1\| | |
| | | | | hypothetical | |
| | | | | protein [Zea | |
| | | | | mays] | |
| 923 | 890 | Zea mays | 1 | hypothetical | 212274814 |
| | | | | protein | |
| | | | | LOC100191388 | |
| | | | | [Zea | |
| | | | | mays] | |
| | | | | &gt; gi\|194688768\| | |
| | | | | gb\|ACF78468.1\| | |
| | | | | unknown | |
| | | | | [Zea mays] | |
| 924 | 891 | Oryza | 0.7869822 | Os09g0135400 | 115478085 |
| | | sativa | | [Oryza | |
| | | Japonica | | sativa | |
| | | Group | | Japonica | |
| | | | | Group] | |
| | | | | &gt; gi\|47848428\| | |
| | | | | dbj\|BAD22285.1\| | |
| | | | | putative | |
| | | | | octicosapeptide/ | |
| | | | | Phox/Bem1p | |
| | | | | (PB1) | |
| | | | | domain- | |
| | | | | containing | |
| | | | | protein | |
| | | | | [Oryza sativa | |
| | | | | Japonica | |
| | | | | Group] | |
| | | | | &gt; gi\|113630871\| | |
| | | | | dbj\|BAF24552.1\| | |
| | | | | Os09g0135400 | |
| | | | | [Oryza | |
| | | | | sativa | |
| | | | | Japonica | |
| | | | | Group] | |
| | 892 | Sorghum | 1 | hypothetical | 255761094 |
| | | bicolor | | protein | |
| | | | | SORBIDRAFT_02g037770 | |
| | | | | [Sorghum | |
| | | | | bicolor] | |
| | | | | &gt; gi\|241924313\| | |
| | | | | gb\|EER97457.1\| | |
| | | | | hypothetical | |
| | | | | protein | |
| | | | | SORBIDRAFT_02g037770 | |
| | | | | [Sorghum | |
| | | | | bicolor] | |
| 925 | 893 | Zea mays | 0.8738462 | hypothetical | 226507742 |
| | | | | protein | |
| | | | | LOC100279098 | |
| | | | | [Zea | |
| | | | | mays] | |
| | | | | &gt; gi\|195658887\| | |
| | | | | gb\|ACG48911.1\| | |
| | | | | hypothetical | |
| | | | | protein [Zea | |
| | | | | mays] | |
| 926 | 894 | Zea mays | 0.8307692 | hypothetical | 226495966 |
| | | | | protein | |
| | | | | LOC100278263 | |
| | | | | [Zea | |
| | | | | mays] | |
| | | | | &gt; gi\|195650593\| | |
| | | | | gb\|ACG44764.1\| | |
| | | | | hypothetical | |
| | | | | protein [Zea | |
| | | | | mays] | |

| | Protein | | | | |
|---|---|---|---|---|---|
| Nucleotide | Sequence | Homolog | miR | | |
| Sequence | seq id | NCBI | Binding | miR | miR |
| seq id no: | no: | Accession | Position | sequence | name |
| 895 | 854 | NP_001170533 | 105-125 | AGGATG | Predicted |
| | | | | CTGACG | zma |
| | | | | CAATGG | mir |
| | | | | GAT/9 | 48486 |
| | 855 | AAN40030 | 33-54 | AGGATG | Predicted |
| | | | | TGAGGC | zma |
| | | | | TATTGG | mir |
| | | | | GGAC/6 | 48492 |
| 896 | 856 | ADU32889 | 1977-1997 | TTAGAT | zma- |
| | | | | GACCAT | miR827 |
| | | | | CAGCAA | |
| | | | | ACA/10 | |
| 897 | 857 | ABC69154 | | | |
| 898 | 858 | NP_001105530 | | | |
| 899 | 859 | ABB77210 | | | |
| 900 | 860 | AAX78232 | | | |
| 901 | 861 | NP_001062825 | | | |
| | 862 | BAD36510 | | | |
| 902 | 863 | ADU32890 | | | |
| 903 | 864 | BAJ99275 | | | |
| 904 | 865 | BAD99131 | | | |
| 905 | 866 | NP_001105530 | 1748-1768 | | |
| 906 | 867 | ABC69154 | | | |
| 907 | 868 | ADU32889 | | | |
| 908 | 869 | ABB77210 | | | |
| 909 | 870 | AAX78232 | | | |
| 910 | 871 | NP_001062825 | | | |
| | 872 | BAD36510 | | | |
| 911 | 873 | ADU32890 | | | |
| 912 | 874 | BAJ99275 | | | |
| 913 | 875 | BAD99131 | | | |
| | 876 | XP_002454482 | 580-600 | | |
| 914 | 877 | ACN30672 | | | |
| 915 | 878 | NP_001183461 | | | |
| | 879 | Q6EPQ3 | | | |
| 916 | 880 | BAJ95234 | | | |
| | 881 | CAD41659 | | | |
| 917 | 882 | NP_001053611 | | | |
| 918 | 883 | CAH66957 | | | |
| | 884 | XP_002446964 | | | |
| 919 | 885 | XP_002282540 | | | |
| | 886 | XP_002462548 | 965-985 | | |
| 920 | 887 | NP_001145770 | | | |
| 921 | 888 | ACN34477 | | | |
| 922 | 889 | NP_001145176 | | | |
| 923 | 890 | NP_001130294 | 1075-1095 | | |
| 924 | 891 | NP_001062638 | | | |
| | 892 | XP_002460936 | 547-567 | ATTCAC | mtr- |
| | | | | GGGGAC | miR2647a |
| | | | | GAACCT | |
| | | | | CCT/8 | |
| 925 | 893 | NP_001145615 | | | |
| 926 | 894 | NP_001145067 | | | |

TABLE 6
Target Genes of Small RNA Molecules that are down regulated during
NUE.

| | Protein | | | | Nucleotide |
|---|---|---|---|---|---|
| Nucleotide | seq id | | % | | NCBI GI |
| seq id no: | no: | Organism | Identity | Annotation | number |
| | 927 | Sorghum | 1 | hypothetical | 255761094 |
| | | bicolor | | protein | |
| | | | | SORBIDRAFT_01g008450 | |
| | | | | [Sorghum | |
| | | | | bicolor] | |
| | | | | &gt; gi\|241917750\| | |
| | | | | gb\|EER90894.1\| | |
| | | | | hypothetical | |
| | | | | protein | |
| | | | | SORBIDRAFT_01g008450 | |
| | | | | [Sorghum | |
| | | | | bicolor] | |
| 1022 | 928 | Zea mays | 0.946721311 | unknown | 223949050 |
| | | | | [Zea mays] | |
| 1023 | 929 | Zea mays | 0.954918033 | unknown | 224029894 |
| | | | | [Zea mays] | |
| 1024 | 930 | Zea mays | 0.942622951 | bifunctional | 195651448 |
| | | | | 3- | |
| | | | | phosphoadenosine | |
| | | | | 5- | |
| | | | | phosphosulfate | |
| | | | | synthetase | |
| | | | | [Zea mays] | |
| 1025 | 931 | Zea mays | 0.946721311 | ATP | 162463127 |
| | | | | sulfurylase | |
| | | | | [Zea mays] | |
| | | | | &gt; gi\|2738750\| | |
| | | | | gb\|AAB94542.1\| | |
| | | | | ATP | |
| | | | | sulfurylase | |
| | | | | [Zea mays] | |
| | 932 | Oryza | 0.799180328 | hypothetical | 54362548 |
| | | sativa | | protein | |
| | | Indica | | OsI_13470 | |
| | | Group | | [Oryza | |
| | | | | sativa Indica | |
| | | | | Group] | |
| 1026 | 933 | Oryza | 0.797131148 | Os03g0743900 | 115455266 |
| | | sativa | | [Oryza | |
| | | Japonica | | sativa | |
| | | Group | | Japonica | |
| | | | | Group] | |
| | | | | &gt; gi\|30017582\| | |
| | | | | gb\|AAP13004.1\| | |
| | | | | putative | |
| | | | | ATP | |
| | | | | sulfurylase | |
| | | | | [Oryza | |
| | | | | sativa | |
| | | | | Japonica | |
| | | | | Group] | |
| | | | | &gt; gi\|108711024\| | |
| | | | | gb\|ABF98819.1\| | |
| | | | | Bifunctional | |
| | | | | 3&apos; - | |
| | | | | phosphoadenosine | |
| | | | | 5&apos; - | |
| | | | | phosphosulfate | |
| | | | | synthethase, | |
| | | | | putative, | |
| | | | | expressed | |
| | | | | [Oryza | |
| | | | | sativa | |
| | | | | Japonica | |
| | | | | Group] | |
| | | | | &gt; gi\|113549705\| | |
| | | | | dbj\|BAF13148.1\| | |
| | | | | Os03g0743900 | |
| | | | | [Oryza | |
| | | | | sativa | |
| | | | | Japonica | |
| | | | | Group] | |
| | | | | &gt; gi\|215704581\| | |
| | | | | dbj\|BAG94214.1\| | |
| | | | | unnamed | |
| | | | | protein | |
| | | | | product | |
| | | | | [Oryza | |
| | | | | sativa | |
| | | | | Japonica | |
| | | | | Group] | |
| 1027 | 934 | Hordeum | 0.793032787 | predicted | 326491124 |
| | | vulgare | | protein | |
| | | subsp. | | [Hordeum | |
| | | vulgare | | vulgare | |
| | | | | subsp. | |
| | | | | vulgare] | |
| | | | | &gt; gi\|326502564\| | |
| | | | | dbj\|BAJ95345.1\| | |
| | | | | predicted | |
| | | | | protein | |
| | | | | [Hordeum | |
| | | | | vulgare | |
| | | | | subsp. | |
| | | | | vulgare] | |
| 1028 | 935 | Oryza | 0.797131148 | plastidic | 3986152 |
| | | sativa | | ATP | |
| | | Indica | | sulfurylase | |
| | | Group | | [Oryza | |
| | | | | sativa Indica | |
| | | | | Group] | |
| | 936 | Oryza | 0.770491803 | hypothetical | 54398660 |
| | | sativa | | protein | |
| | | Japonica | | OsJ_12530 | |
| | | Group | | [Oryza | |
| | | | | sativa | |
| | | | | Japonica | |
| | | | | Group] | |
| | 937 | Sorghum | 1 | hypothetical | 255761094 |
| | | bicolor | | protein | |
| | | | | SORBIDRAFT_08g004650 | |
| | | | | [Sorghum | |
| | | | | bicolor] | |
| | | | | &gt; gi\|241942597\| | |
| | | | | gb\|EES15742.1\| | |
| | | | | hypothetical | |
| | | | | protein | |
| | | | | SORBIDRAFT_08g004650 | |
| | | | | [Sorghum | |
| | | | | bicolor] | |
| 1029 | 938 | Oryza | 0.705440901 | Os12g0174100 | 115487595 |
| | | sativa | | [Oryza | |
| | | Japonica | | sativa | |
| | | Group | | Japonica | |
| | | | | Group] | |
| | | | | &gt; gi\|77553790\| | |
| | | | | gb\|ABA96586.1\| | |
| | | | | Growth | |
| | | | | regulator | |
| | | | | protein, | |
| | | | | putative, | |
| | | | | expressed | |
| | | | | [Oryza | |
| | | | | sativa | |
| | | | | Japonica | |
| | | | | Group] | |
| | | | | &gt; gi\|255670095\| | |
| | | | | dbj\|BAF29304.2\| | |
| | | | | Os12g0174100 | |
| | | | | [Oryza | |
| | | | | sativa | |
| | | | | Japonica | |
| | | | | Group] | |
| | 939 | Oryza | 0.705440901 | hypothetical | 54398660 |
| | | sativa | | protein | |
| | | Japonica | | OsJ_35390 | |
| | | Group | | [Oryza | |
| | | | | sativa | |
| | | | | Japonica | |
| | | | | Group] | |
| | 940 | Oryza | 0.701688555 | hypothetical | 54362548 |
| | | sativa | | protein | |
| | | Indica | | OsI_37646 | |
| | | Group | | [Oryza | |
| | | | | sativa Indica | |
| | | | | Group] | |
| 1030 | 941 | Zea mays | 1 | unknown | 224029894 |
| | | | | [Zea mays] | |
| 1031 | 942 | Zea mays | 0.983640082 | ATP | 162463127 |
| | | | | sulfurylase | |
| | | | | [Zea mays] | |
| | | | | &gt; gi\|2738750\| | |
| | | | | gb\|AAB94542.1\| | |
| | | | | ATP | |
| | | | | sulfurylase | |
| | | | | [Zea mays] | |
| 1032 | 943 | Zea mays | 0.940695297 | unknown | 223949050 |
| | | | | [Zea mays] | |
| 1033 | 944 | Zea mays | 0.936605317 | bifunctional | 195651448 |
| | | | | 3- | |
| | | | | phosphoadenosine | |
| | | | | 5- | |
| | | | | phosphosulfate | |
| | | | | synthetase | |
| | | | | [Zea mays] | |
| | 945 | Sorghum | 0.938650307 | hypothetical | 255761094 |
| | | bicolor | | protein | |
| | | | | SORBIDRAFT_01g008450 | |
| | | | | [Sorghum | |
| | | | | bicolor] | |
| | | | | &gt; gi\|241917750\| | |
| | | | | gb\|EER90894.1\| | |
| | | | | hypothetical | |
| | | | | protein | |
| | | | | SORBIDRAFT_01g008450 | |
| | | | | [Sorghum | |
| | | | | bicolor] | |
| 1034 | 946 | Hordeum | 0.842535787 | predicted | 326491124 |
| | | vulgare | | protein | |
| | | subsp. | | [Hordeum | |
| | | vulgare | | vulgare | |
| | | | | subsp. | |
| | | | | vulgare] | |
| | | | | &gt; gi\|326502564\| | |
| | | | | dbj\|BAJ95345.1\| | |
| | | | | predicted | |
| | | | | protein | |
| | | | | [Hordeum | |
| | | | | vulgare | |
| | | | | subsp. | |
| | | | | vulgare] | |
| | 947 | Oryza | 0.795501022 | hypothetical | 54362548 |
| | | sativa | | protein | |
| | | Indica | | OsI_13470 | |
| | | Group | | [Oryza | |
| | | | | sativa Indica | |
| | | | | Group] | |
| 1035 | 948 | Oryza | 0.793456033 | Os03g0743900 | 115455266 |
| | | sativa | | [Oryza | |
| | | Japonica | | sativa | |
| | | Group | | Japonica | |
| | | | | Group] | |
| | | | | &gt; gi\|30017582\| | |
| | | | | gb\|AAP13004.1\| | |
| | | | | putative | |
| | | | | ATP | |
| | | | | sulfurylase | |
| | | | | [Oryza | |
| | | | | sativa | |
| | | | | Japonica | |
| | | | | Group] | |
| | | | | &gt; gi\|108711024\| | |
| | | | | gb\|ABF98819.1\| | |
| | | | | Bifunctional | |
| | | | | 3&apos; - | |
| | | | | phosphoadenosine | |
| | | | | 5&apos; - | |
| | | | | phosphosulfate | |
| | | | | synthethase, | |
| | | | | putative, | |
| | | | | expressed | |
| | | | | [Oryza | |
| | | | | sativa | |
| | | | | Japonica | |
| | | | | Group] | |
| | | | | &gt; gi\|113549705\| | |
| | | | | dbj\|BAF13148.1\| | |
| | | | | Os03g0743900 | |
| | | | | [Oryza | |
| | | | | sativa | |
| | | | | Japonica | |
| | | | | Group] | |
| | | | | &gt; gi\|215704581\| | |
| | | | | dbj\|BAG94214.1\| | |
| | | | | unnamed | |
| | | | | protein | |
| | | | | product | |
| | | | | [Oryza | |
| | | | | sativa | |
| | | | | Japonica | |
| | | | | Group] | |
| 1036 | 949 | Oryza | 0.793456033 | plastidic | 3986152 |
| | | sativa | | ATP | |
| | | Indica | | sulfurylase | |
| | | Group | | [Oryza | |
| | | | | sativa Indica | |
| | | | | Group] | |
| | 950 | Oryza | 0.764826176 | hypothetical | 54398660 |
| | | sativa | | protein | |
| | | Japonica | | OsJ_12530 | |
| | | Group | | [Oryza | |
| | | | | sativa | |
| | | | | Japonica | |
| | | | | Group] | |
| | 951 | Sorghum | 1 | hypothetical | 255761094 |
| | | bicolor | | protein | |
| | | | | SORBIDRAFT_04g026710 | |
| | | | | [Sorghum | |
| | | | | bicolor] | |
| | | | | &gt; gi\|241932317\| | |
| | | | | gb\|EES05462.1\| | |
| | | | | hypothetical | |
| | | | | protein | |
| | | | | SORBIDRAFT_04g026710 | |
| | | | | [Sorghum | |
| | | | | bicolor] | |
| 1037 | 952 | Zea mays | 0.880208333 | unknown | 223974072 |
| | | | | [Zea mays] | |
| 1038 | 953 | Zea mays | 0.880208333 | hypothetical | 226500051 |
| | | | | protein | |
| | | | | LOC100276301 | |
| | | | | [Zea | |
| | | | | mays] | |
| | | | | &gt; gi\|195623072\| | |
| | | | | gb\|ACG33366.1\| | |
| | | | | hypothetical | |
| | | | | protein [Zea | |
| | | | | mays] | |
| 1039 | 954 | Zea mays | 0.864583333 | hypothetical | 226492590 |
| | | | | protein | |
| | | | | LOC100277041 | |
| | | | | [Zea | |
| | | | | mays] | |
| | | | | &gt; gi\|195638130\| | |
| | | | | gb\|ACG38533.1\| | |
| | | | | hypothetical | |
| | | | | protein [Zea | |
| | | | | mays] | |
| | | | | &gt; gi\|223942145\| | |
| | | | | gb\|ACN25156.1\| | |
| | | | | unknown | |
| | | | | [Zea mays] | |
| 1040 | 955 | Oryza | 0.776041667 | Os02g0631000 | 115447434 |
| | | sativa | | [Oryza | |
| | | Japonica | | sativa | |
| | | Group | | Japonica | |
| | | | | Group] | |
| | | | | &gt; gi\|49389184\| | |
| | | | | dbj\|BAD26474.1\| | |
| | | | | unknown | |
| | | | | protein | |
| | | | | [Oryza | |
| | | | | sativa | |
| | | | | Japonica | |
| | | | | Group] | |
| | | | | &gt; gi\|113537028\| | |
| | | | | dbj\|BAF09411.1\| | |
| | | | | Os02g0631000 | |
| | | | | [Oryza | |
| | | | | sativa | |
| | | | | Japonica | |
| | | | | Group] | |
| | | | | &gt; gi\|215697023\| | |
| | | | | dbj\|BAG91017.1\| | |
| | | | | unnamed | |
| | | | | protein | |
| | | | | product | |
| | | | | [Oryza | |
| | | | | sativa | |
| | | | | Japonica | |
| | | | | Group] | |
| | | | | &gt; gi\|218191219\| | |
| | | | | gb\|EEC73646.1\| | |
| | | | | hypothetical | |
| | | | | protein | |
| | | | | OsI_08167 | |
| | | | | [Oryza | |
| | | | | sativa Indica | |
| | | | | Group] | |
| | | | | &gt; gi\|222623287\| | |
| | | | | gb\|EEE57419.1\| | |
| | | | | hypothetical | |
| | | | | protein | |
| | | | | OsJ_07614 | |
| | | | | [Oryza | |
| | | | | sativa | |
| | | | | Japonica | |
| | | | | Group] | |
| 1041 | 956 | Hordeum | 0.760416667 | predicted | 326512283 |
| | | vulgare | | protein | |
| | | subsp. | | [Hordeum | |
| | | vulgare | | vulgare | |
| | | | | subsp. | |
| | | | | vulgare] | |
| | | | | &gt; gi\|326519272\| | |
| | | | | dbj\|BAJ96635.1\| | |
| | | | | predicted | |
| | | | | protein | |
| | | | | [Hordeum | |
| | | | | vulgare | |
| | | | | subsp. | |
| | | | | vulgare] | |
| 1042 | 957 | Zea mays | 1 | AP2 domain | 148964889 |
| | | | | transcription | |
| | | | | factor [Zea | |
| | | | | mays] | |
| 1043 | 958 | Zea mays | 0.96043956 | AP2 domain | 148964859 |
| | | | | transcription | |
| | | | | factor [Zea | |
| | | | | mays] | |
| | 959 | Sorghum | 1 | hypothetical | 255761094 |
| | | bicolor | | protein | |
| | | | | SORBIDRAFT_02g007000 | |
| | | | | [Sorghum | |
| | | | | bicolor] | |
| | | | | &gt; gi\|241922957\| | |
| | | | | gb\|EER96101.1\| | |
| | | | | hypothetical | |
| | | | | protein | |
| | | | | SORBIDRAFT_02g007000 | |
| | | | | [Sorghum | |
| | | | | bicolor] | |
| 1044 | 960 | Zea mays | 0.85528757 | sister of | 225703093 |
| | | | | indeterminate | |
| | | | | spikelet 1 | |
| | | | | [Zea mays] | |
| | | | | &gt; gi\|223947941\| | |
| | | | | gb\|ACN28054.1\| | |
| | | | | unknown | |
| | | | | [Zea mays] | |
| 1045 | 961 | Zea mays | 0.844155844 | sister of | 224579291 |
| | | | | indeterminate | |
| | | | | spikelet 1 | |
| | | | | [Zea mays] | |
| 1046 | 962 | Zea mays | 0.742115028 | floral | 195653672 |
| | | | | homeotic | |
| | | | | protein [Zea | |
| | | | | mays] | |
| | | | | &gt; gi\|238015134\| | |
| | | | | gb\|ACR38602.1\| | |
| | | | | unknown | |
| | | | | [Zea mays] | |
| 1047 | 963 | Oryza | 1 | Os01g0834500 | 115440880 |
| | | sativa | | [Oryza | |
| | | Japonica | | sativa | |
| | | Group | | Japonica | |
| | | | | Group] | |
| | | | | &gt; gi\|115456215\| | |
| | | | | ref\|NP_001051708.1\| | |
| | | | | Os03g0818400 | |
| | | | | [Oryza | |
| | | | | sativa | |
| | | | | Japonica | |
| | | | | Group] | |
| | | | | &gt; gi\|297720551\| | |
| | | | | ref\|NP_001172637.1\| | |
| | | | | Os01g0834601 | |
| | | | | [Oryza | |
| | | | | sativa | |
| | | | | Japonica | |
| | | | | Group] | |
| | | | | &gt; gi\|313103637\| | |
| | | | | pdb\|3IZ6\| | |
| | | | | L Chain | |
| | | | | L, | |
| | | | | Localization | |
| | | | | Of The | |
| | | | | Small | |
| | | | | Subunit | |
| | | | | Ribosomal | |
| | | | | Proteins Into | |
| | | | | A 5.5 A | |
| | | | | Cryo-Em | |
| | | | | Map Of | |
| | | | | Triticum | |
| | | | | Aestivum | |
| | | | | Translating | |
| | | | | 80s | |
| | | | | Ribosome | |
| | | | | &gt; gi\|20805266\| | |
| | | | | dbj\|BAB92932.1\| | |
| | | | | putative 40s | |
| | | | | ribosomal | |
| | | | | protein S23 | |
| | | | | [Oryza | |
| | | | | sativa | |
| | | | | Japonica | |
| | | | | Group] | |
| | | | | &gt; gi\|20805267\| | |
| | | | | dbj\|BAB92933.1\| | |
| | | | | putative 40s | |
| | | | | ribosomal | |
| | | | | protein S23 | |
| | | | | [Oryza | |
| | | | | sativa | |
| | | | | Japonica | |
| | | | | Group] | |
| | | | | &gt; gi\|21671347\| | |
| | | | | dbj\|BAC02683.1\| | |
| | | | | putative 40s | |
| | | | | ribosomal | |
| | | | | protein S23 | |
| | | | | [Oryza | |
| | | | | sativa | |
| | | | | Japonica | |
| | | | | Group] | |
| | | | | &gt; gi\|21671348\| | |
| | | | | dbj\|BAC02684.1\| | |
| | | | | putative 40s | |
| | | | | ribosomal | |
| | | | | protein S23 | |
| | | | | [Oryza | |
| | | | | sativa | |
| | | | | Japonica | |
| | | | | Group] | |
| | | | | &gt; gi\|28876025\| | |
| | | | | gb\|AAO60034.1\| | |
| | | | | 40S | |
| | | | | ribosomal | |
| | | | | protein S23 | |
| | | | | [Oryza | |
| | | | | sativa | |
| | | | | Japonica | |
| | | | | Group] | |
| | | | | &gt; gi\|29124115\| | |
| | | | | gb\|AAO65856.1\| | |
| | | | | 40S | |
| | | | | ribosomal | |
| | | | | protein S23 | |
| | | | | [Oryza | |
| | | | | sativa | |
| | | | | Japonica | |
| | | | | Group] | |
| | | | | &gt; gi\|108711771\| | |
| | | | | gb\|ABF99566.1\| | |
| | | | | 40S | |
| | | | | ribosomal | |
| | | | | protein S23, | |
| | | | | putative, | |
| | | | | expressed | |
| | | | | [Oryza | |
| | | | | sativa | |
| | | | | Japonica | |
| | | | | Group] | |
| | | | | &gt; gi\|113534251\| | |
| | | | | dbj\|BAF06634.1\| | |
| | | | | Os01g0834500 | |
| | | | | [Oryza | |
| | | | | sativa | |
| | | | | Japonica | |
| | | | | Group] | |
| | | | | &gt; gi\|113550179\| | |
| | | | | dbj\|BAF13622.1\| | |
| | | | | Os03g0818400 | |
| | | | | [Oryza | |
| | | | | sativa | |
| | | | | Japonica | |
| | | | | Group] | |
| | | | | &gt; gi\|125528286\| | |
| | | | | gb\|EAY76400.1\| | |
| | | | | hypothetical | |
| | | | | protein | |
| | | | | OsI_04329 | |
| | | | | [Oryza | |
| | | | | sativa Indica | |
| | | | | Group] | |
| | | | | &gt; gi\|125546216\| | |
| | | | | gb\|EAY92355.1\| | |
| | | | | hypothetical | |
| | | | | protein | |
| | | | | OsI_14082 | |
| | | | | [Oryza | |
| | | | | sativa Indica | |
| | | | | Group] | |
| | | | | &gt; gi\|215697420\| | |
| | | | | dbj\|BAG91414.1\| | |
| | | | | unnamed | |
| | | | | protein | |
| | | | | product | |
| | | | | [Oryza | |
| | | | | sativa | |
| | | | | Japonica | |
| | | | | Group] | |
| | | | | &gt; gi\|215734943\| | |
| | | | | dbj\|BAG95665.1\| | |
| | | | | unnamed | |
| | | | | protein | |
| | | | | product | |
| | | | | [Oryza | |
| | | | | sativa | |
| | | | | Japonica | |
| | | | | Group] | |
| | | | | &gt; gi\|255673847\| | |
| | | | | dbj\|BAH91367.1\| | |
| | | | | Os01g0834601 | |
| | | | | [Oryza | |
| | | | | sativa | |
| | | | | Japonica | |
| | | | | Group] | |
| | | | | &gt; gi\|326501134\| | |
| | | | | dbj\|BAJ98798.1\| | |
| | | | | predicted | |
| | | | | protein | |
| | | | | [Hordeum | |
| | | | | vulgare | |
| | | | | subsp. | |
| | | | | vulgare] | |
| | | | | &gt; gi\|326506086\| | |
| | | | | dbj\|BAJ91282.1\| | |
| | | | | predicted | |
| | | | | protein | |
| | | | | [Hordeum | |
| | | | | vulgare | |
| | | | | subsp. | |
| | | | | vulgare] | |
| 1048 | 964 | Zea mays | 0.992957746 | hypothetical | 212722729 |
| | | | | protein | |
| | | | | LOC100192600 | |
| | | | | [Zea | |
| | | | | mays] | |
| | | | | &gt; gi\|242032479\| | |
| | | | | ref\|XP_002463634.1\| | |
| | | | | hypothetical | |
| | | | | protein | |
| | | | | SORBIDRAFT_01g003410 | |
| | | | | [Sorghum | |
| | | | | bicolor] | |
| | | | | &gt; gi\|242059153\| | |
| | | | | ref\|XP_002458722.1\| | |
| | | | | hypothetical | |
| | | | | protein | |
| | | | | SORBIDRAFT_03g039010 | |
| | | | | [Sorghum | |
| | | | | bicolor] | |
| | | | | &gt; gi\|242090801\| | |
| | | | | ref\|XP_002441233.1\| | |
| | | | | hypothetical | |
| | | | | protein | |
| | | | | SORBIDRAFT_09g022840 | |
| | | | | [Sorghum | |
| | | | | bicolor] | |
| | | | | &gt; gi\|194691088\| | |
| | | | | gb\|ACF79628.1\| | |
| | | | | unknown | |
| | | | | [Zea mays] | |
| | | | | &gt; gi\|194697612\| | |
| | | | | gb\|ACF82890.1\| | |
| | | | | unknown | |
| | | | | [Zea mays] | |
| | | | | &gt; gi\|194702740\| | |
| | | | | gb\|ACF85454.1\| | |
| | | | | unknown | |
| | | | | [Zea mays] | |
| | | | | &gt; gi\|195606082\| | |
| | | | | gb\|ACG24871.1\| | |
| | | | | 40S | |
| | | | | ribosomal | |
| | | | | protein S23 | |
| | | | | [Zea mays] | |
| | | | | &gt; gi\|195618728\| | |
| | | | | gb\|ACG31194.1\| | |
| | | | | 40S | |
| | | | | ribosomal | |
| | | | | protein S23 | |
| | | | | [Zea mays] | |
| | | | | &gt; gi\|195619636\| | |
| | | | | gb\|ACG31648.1\| | |
| | | | | 40S | |
| | | | | ribosomal | |
| | | | | protein S23 | |
| | | | | [Zea mays] | |
| | | | | &gt; gi\|195625318\| | |
| | | | | gb\|ACG34489.1\| | |
| | | | | 40S | |
| | | | | ribosomal | |
| | | | | protein S23 | |
| | | | | [Zea mays] | |
| | | | | &gt; gi\|195628702\| | |
| | | | | gb\|ACG36181.1\| | |
| | | | | 40S | |
| | | | | ribosomal | |
| | | | | protein S23 | |
| | | | | [Zea mays] | |
| | | | | &gt; gi\|195657679\| | |
| | | | | gb\|ACG48307.1\| | |
| | | | | 40S | |
| | | | | ribosomal | |
| | | | | protein S23 | |
| | | | | [Zea mays] | |
| | | | | &gt; gi\|238012290\| | |
| | | | | gb\|ACR37180.1\| | |
| | | | | unknown | |
| | | | | [Zea mays] | |
| | | | | &gt; gi\|241917488\| | |
| | | | | gb\|EER90632.1\| | |
| | | | | hypothetical | |
| | | | | protein | |
| | | | | SORBIDRAFT_01g003410 | |
| | | | | [Sorghum | |
| | | | | bicolor] | |
| | | | | &gt; gi\|241930697\| | |
| | | | | gb\|EES03842.1\| | |
| | | | | hypothetical | |
| | | | | protein | |
| | | | | SORBIDRAFT_03g039010 | |
| | | | | [Sorghum | |
| | | | | bicolor] | |
| | | | | &gt; gi\|241946518\| | |
| | | | | gb\|EES19663.1\| | |
| | | | | hypothetical | |
| | | | | protein | |
| | | | | SORBIDRAFT_09g022840 | |
| | | | | [Sorghum | |
| | | | | bicolor] | |
| 1049 | 965 | Zea mays | 0.985915493 | 40S | 195622025 |
| | | | | ribosomal | |
| | | | | protein S23 | |
| | | | | [Zea mays] | |
| 1050 | 966 | Elaeis | 0.978873239 | 40S | 192910819 |
| | | guineensis | | ribosomal | |
| | | | | protein S23 | |
| | | | | [Elaeis | |
| | | | | guineensis] | |
| | | | | &gt; gi\|192910894\| | |
| | | | | gb\|ACF06555.1\| | |
| | | | | 40S | |
| | | | | ribosomal | |
| | | | | protein S23 | |
| | | | | [Elaeis | |
| | | | | guineensis] | |
| 1051 | 967 | Elaeis | 0.971830986 | 40S | 192910821 |
| | | guineensis | | ribosomal | |
| | | | | protein S23 | |
| | | | | [Elaeis | |
| | | | | guineensis] | |
| 1052 | 968 | Solanum | 0.964788732 | unknown | 77999292 |
| | | tuberosum | | [Solanum | |
| | | | | tuberosum] | |
| | 969 | Ricinus | 0.964788732 | 40S | 255761086 |
| | | communis | | ribosomal | |
| | | | | protein S23, | |
| | | | | putative | |
| | | | | [Ricinus | |
| | | | | communis] | |
| | | | | &gt; gi\|255568414\| | |
| | | | | ref\|XP_002525181.1\| | |
| | | | | 40S | |
| | | | | ribosomal | |
| | | | | protein S23, | |
| | | | | putative | |
| | | | | [Ricinus | |
| | | | | communis] | |
| | | | | &gt; gi\|223535478\| | |
| | | | | gb\|EEF37147.1\| | |
| | | | | 40S | |
| | | | | ribosomal | |
| | | | | protein S23, | |
| | | | | putative | |
| | | | | [Ricinus | |
| | | | | communis] | |
| | | | | &gt; gi\|223536832\| | |
| | | | | gb\|EEF38471.1\| | |
| | | | | 40S | |
| | | | | ribosomal | |
| | | | | protein S23, | |
| | | | | putative | |
| | | | | [Ricinus | |
| | | | | communis] | |
| 1053 | 970 | Vitis | 0.964788732 | PREDICTED: | 225439887 |
| | | vinifera | | hypothetical | |
| | | | | protein | |
| | | | | [Vitis | |
| | | | | vinifera] | |
| 1054 | 971 | Zea mays | 1 | unknown | 223972764 |
| | | | | [Zea mays] | |
| | | | | &gt; gi\|223973927\| | |
| | | | | gb\|ACN31151.1\| | |
| | | | | unknown | |
| | | | | [Zea mays] | |
| | | | | &gt; gi\|323388595\| | |
| | | | | gb\|ADX60102.1\| | |
| | | | | SBP | |
| | | | | transcription | |
| | | | | factor [Zea | |
| | | | | mays] | |
| 1055 | 972 | Zea mays | 0.984615385 | hypothetical | 226530074 |
| | | | | protein | |
| | | | | LOC100278824 | |
| | | | | [Zea | |
| | | | | mays] | |
| | | | | &gt; gi\|195656399\| | |
| | | | | gb\|ACG47667.1\| | |
| | | | | hypothetical | |
| | | | | protein [Zea | |
| | | | | mays] | |
| | 973 | Sorghum | 0.870769231 | hypothetical | 255761094 |
| | | bicolor | | protein | |
| | | | | SORBIDRAFT_05g017510 | |
| | | | | [Sorghum | |
| | | | | bicolor] | |
| | | | | &gt; gi\|241936618\| | |
| | | | | gb\|EES09763.1\| | |
| | | | | hypothetical | |
| | | | | protein | |
| | | | | SORBIDRAFT_05g017510 | |
| | | | | [Sorghum | |
| | | | | bicolor] | |
| | 974 | Sorghum | 1 | hypothetical | 255761094 |
| | | bicolor | | protein | |
| | | | | SORBIDRAFT_03g025410 | |
| | | | | [Sorghum | |
| | | | | bicolor] | |
| | | | | &gt; gi\|241927774\| | |
| | | | | gb\|EES00919.1\| | |
| | | | | hypothetical | |
| | | | | protein | |
| | | | | SORBIDRAFT_03g025410 | |
| | | | | [Sorghum | |
| | | | | bicolor] | |
| 1056 | 975 | Zea mays | 0.893939394 | unknown | 223946882 |
| | | | | [Zea mays] | |
| 1057 | 976 | Zea mays | 0.890151515 | hypothetical | 226501393 |
| | | | | protein | |
| | | | | LOC100278489 | |
| | | | | [Zea | |
| | | | | mays] | |
| | | | | &gt; gi\|195653155\| | |
| | | | | gb\|ACG46045.1\| | |
| | | | | hypothetical | |
| | | | | protein [Zea | |
| | | | | mays] | |
| 1058 | 977 | Zea mays | 1 | unknown | 238908852 |
| | | | | [Zea mays] | |
| | | | | &gt; gi\|323388573\| | |
| | | | | gb\|ADX60091.1\| | |
| | | | | SBP | |
| | | | | transcription | |
| | | | | factor [Zea | |
| | | | | mays] | |
| 1059 | 978 | Zea mays | 0.997354497 | squamosa | 195651290 |
| | | | | promoter- | |
| | | | | binding-like | |
| | | | | protein 9 | |
| | | | | [Zea mays] | |
| | 979 | Sorghum | 0.828042328 | hypothetical | 255761094 |
| | | bicolor | | protein | |
| | | | | SORBIDRAFT_02g028420 | |
| | | | | [Sorghum | |
| | | | | bicolor] | |
| | | | | &gt; gi\|241925948\| | |
| | | | | gb\|EER99092.1\| | |
| | | | | hypothetical | |
| | | | | protein | |
| | | | | SORBIDRAFT_02g028420 | |
| | | | | [Sorghum | |
| | | | | bicolor] | |
| 1060 | 980 | Zea mays | 0.756613757 | hypothetical | 219363104 |
| | | | | protein | |
| | | | | LOC100217104 | |
| | | | | [Zea | |
| | | | | mays] | |
| | | | | &gt; gi\|194697718\| | |
| | | | | gb\|ACF82943.1\| | |
| | | | | unknown | |
| | | | | [Zea mays] | |
| 1061 | 981 | Zea mays | 1 | squamosa | 226529809 |
| | | | | promoter- | |
| | | | | binding-like | |
| | | | | protein 11 | |
| | | | | [Zea mays] | |
| | | | | &gt; gi\|195627850\| | |
| | | | | gb\|ACG35755.1\| | |
| | | | | squamosa | |
| | | | | promoter- | |
| | | | | binding-like | |
| | | | | protein 11 | |
| | | | | [Zea mays] | |
| | | | | &gt; gi\|195644948\| | |
| | | | | gb\|ACG41942.1\| | |
| | | | | squamosa | |
| | | | | promoter- | |
| | | | | binding-like | |
| | | | | protein 11 | |
| | | | | [Zea mays] | |
| | 982 | Sorghum | 0.876993166 | hypothetical | 255761094 |
| | | bicolor | | protein | |
| | | | | SORBIDRAFT_10g029190 | |
| | | | | [Sorghum | |
| | | | | bicolor] | |
| | | | | &gt; gi\|241917194\| | |
| | | | | gb\|EER90338.1\| | |
| | | | | hypothetical | |
| | | | | protein | |
| | | | | SORBIDRAFT_10g029190 | |
| | | | | [Sorghum | |
| | | | | bicolor] | |
| 1062 | 983 | Zea mays | 1 | hypothetical | 219363104 |
| | | | | protein | |
| | | | | LOC100217104 | |
| | | | | [Zea | |
| | | | | mays] | |
| | | | | &gt; gi\|194697718\| | |
| | | | | gb\|ACF82943.1\| | |
| | | | | unknown | |
| | | | | [Zea mays] | |
| | 984 | Sorghum | 0.817232376 | hypothetical | 255761094 |
| | | bicolor | | protein | |
| | | | | SORBIDRAFT_02g028420 | |
| | | | | [Sorghum | |
| | | | | bicolor] | |
| | | | | &gt; gi\|241925948\| | |
| | | | | gb\|EER99092.1\| | |
| | | | | hypothetical | |
| | | | | protein | |
| | | | | SORBIDRAFT_02g028420 | |
| | | | | [Sorghum | |
| | | | | bicolor] | |
| 1063 | 985 | Zea mays | 0.759791123 | unknown | 238908852 |
| | | | | [Zea mays] | |
| | | | | &gt; gi\|323388573\| | |
| | | | | gb\|ADX60091.1\| | |
| | | | | SBP | |
| | | | | transcription | |
| | | | | factor [Zea | |
| | | | | mays] | |
| 1064 | 986 | Zea mays | 0.757180157 | squamosa | 195651290 |
| | | | | promoter- | |
| | | | | binding-like | |
| | | | | protein 9 | |
| | | | | [Zea mays] | |
| 1065 | 987 | Zea mays | 1 | SBP-domain | 5931785 |
| | | | | protein 5 | |
| | | | | [Zea mays] | |
| | 988 | Sorghum | 0.854103343 | hypothetical | 255761094 |
| | | bicolor | | protein | |
| | | | | SORBIDRAFT_07g027740 | |
| | | | | [Sorghum | |
| | | | | bicolor] | |
| | | | | &gt; gi\|241941121\| | |
| | | | | gb\|EES14266.1\| | |
| | | | | hypothetical | |
| | | | | protein | |
| | | | | SORBIDRAFT_07g027740 | |
| | | | | [Sorghum | |
| | | | | bicolor] | |
| 1066 | 989 | Zea mays | 0.784194529 | unknown | 219885132 |
| | | | | [Zea mays] | |
| 1067 | 990 | Zea mays | 1 | MTA/SAH | 226529725 |
| | | | | nucleosidase | |
| | | | | [Zea mays] | |
| | | | | &gt; gi\|195658647\| | |
| | | | | gb\|ACG48791.1\| | |
| | | | | MTA/SAH | |
| | | | | nucleosidase | |
| | | | | [Zea mays] | |
| | | | | &gt; gi\|223973627\| | |
| | | | | gb\|ACN31001.1\| | |
| | | | | unknown | |
| | | | | [Zea mays] | |
| 1068 | 991 | Zea mays | 0.884462151 | unknown | 194699507 |
| | | | | [Zea mays] | |
| 1069 | 992 | Zea mays | 0.884462151 | MTA/SAH | 195640251 |
| | | | | nucleosidase | |
| | | | | [Zea mays] | |
| | 993 | Sorghum | 0.884462151 | hypothetical | 255761094 |
| | | bicolor | | protein | |
| | | | | SORBIDRA | |
| | | | | FT_07g026190 | |
| | | | | [Sorghum | |
| | | | | bicolor] | |
| | | | | &gt;gi\|241942163\| | |
| | | | | gb\|EES15308.1\| | |
| | | | | hypothetical | |
| | | | | protein | |
| | | | | SORBIDRA | |
| | | | | FT_07g026190 | |
| | | | | [Sorghum | |
| | | | | bicolor] | |
| 1070 | 994 | Zea mays | 0.900398406 | unknown | 223974590 |
| | | | | [Zea mays] | |
| 1071 | 995 | Oryza | 0.796812749 | Os06g0112200 | 115465985 |
| | | sativa | | [Oryza | |
| | | Japonica | | sativa | |
| | | Group | | Japonica | |
| | | | | Group] | |
| | | | | &gt;gi\|7363290\| | |
| | | | | dbj\|BAA93034.1\| | |
| | | | | methylthioadenosine/ | |
| | | | | S- | |
| | | | | adenosyl | |
| | | | | homocysteine | |
| | | | | nucleosidase | |
| | | | | [Oryza | |
| | | | | sativa | |
| | | | | Japonica | |
| | | | | Group] | |
| | | | | &gt;gi\|32352128\| | |
| | | | | dbj\|BAC78557.1\| | |
| | | | | hypothetical | |
| | | | | protein | |
| | | | | [Oryza | |
| | | | | sativa | |
| | | | | Japonica | |
| | | | | Group] | |
| | | | | &gt;gi\|113594632\| | |
| | | | | dbj\|BAF18506.1\| | |
| | | | | Os06g0112200 | |
| | | | | [Oryza | |
| | | | | sativa | |
| | | | | Japonica | |
| | | | | Group] | |
| | | | | &gt;gi\|125595804\| | |
| | | | | gb\|EAZ35584.1\| | |
| | | | | hypothetical | |
| | | | | protein | |
| | | | | OsJ_19870 | |
| | | | | [Oryza | |
| | | | | sativa | |
| | | | | Japonica | |
| | | | | Group] | |
| | | | | &gt;gi\|215694661\| | |
| | | | | dbj\|BAG89852.1\| | |
| | | | | unnamed | |
| | | | | protein | |
| | | | | product | |
| | | | | [Oryza | |
| | | | | sativa | |
| | | | | Japonica | |
| | | | | Group] | |
| | | | | &gt;gi\|215740802\| | |
| | | | | dbj\|BAG96958.1\| | |
| | | | | unnamed | |
| | | | | protein | |
| | | | | product | |
| | | | | [Oryza | |
| | | | | sativa | |
| | | | | Japonica | |
| | | | | Group] | |
| 1072 | 996 | Oryza | 0.792828685 | methylthioadenosine/ | 18087496 |
| | | sativa | | S- | |
| | | | | adenosyl | |
| | | | | homocysteine | |
| | | | | nucleosidase | |
| | | | | [Oryza | |
| | | | | sativa] | |
| 1073 | 997 | Oryza | 0.792828685 | mta/sah | 149390954 |
| | | sativa | | nucleosidase | |
| | | Indica | | [Oryza | |
| | | Group | | sativa Indica | |
| | | | | Group] | |
| 1074 | 998 | Hordeum | 0.780876494 | predicted | 326512819 |
| | | vulgare | | protein | |
| | | subsp. | | [Hordeum | |
| | | vulgare | | vulgare | |
| | | | | subsp. | |
| | | | | vulgare] | |
| | | | | &gt;gi\|326534118\| | |
| | | | | dbj\|BAJ89409.1\| | |
| | | | | predicted | |
| | | | | protein | |
| | | | | [Hordeum | |
| | | | | vulgare | |
| | | | | subsp. | |
| | | | | vulgare] | |
| | 999 | Oryza | 0.784860558 | hypothetical | 54362548 |
| | | sativa | | protein | |
| | | Indica | | OsI_21350 | |
| | | Group | | [Oryza | |
| | | | | sativa Indica | |
| | | | | Group] | |
| 1075 | 1000 | Zea mays | 1 | teosinte | 72536147 |
| | | subsp. | | glume | |
| | | mays | | architecture | |
| | | | | 1 [Zea mays | |
| | | | | subsp. mays] | |
| | 1001 | Zea mays | 0.983796296 | teosinte | |
| | | subsp. | | glume | |
| | | mays | | architecture | |
| | | | | 1 [Zea mays | |
| | | | | subsp. mays] | |
| 1076 | 1002 | Zea mays | 0.990740741 | teosinte | 62467433 |
| | | subsp. | | glume | |
| | | mays | | architecture | |
| | | | | 1 [Zea mays | |
| | | | | subsp. mays] | |
| | | | | &gt;gi\|62467440\| | |
| | | | | gb\|AAX83874.1\| | |
| | | | | teosinte | |
| | | | | glume | |
| | | | | architecture | |
| | | | | 1 [Zea mays | |
| | | | | subsp. mays] | |
| | 1003 | Sorghum | 0.800925926 | hypothetical | 255761094 |
| | | bicolor | | protein | |
| | | | | SORBIDRAFT_07g026220 | |
| | | | | [Sorghum | |
| | | | | bicolor] | |
| | | | | &gt;gi\|241942165\| | |
| | | | | gb\|EES15310.1\| | |
| | | | | hypothetical | |
| | | | | protein | |
| | | | | SORBIDRAFT_07g026220 | |
| | | | | [Sorghum | |
| | | | | bicolor] | |
| | 1004 | Sorghum | 1 | hypothetical | 255761094 |
| | | bicolor | | protein | |
| | | | | SORBIDRAFT_02g038960 | |
| | | | | [Sorghum | |
| | | | | bicolor] | |
| | | | | &gt;gi\|241926544\| | |
| | | | | gb\|EER99688.1\| | |
| | | | | hypothetical | |
| | | | | protein | |
| | | | | SORBIDRAFT_02g038960 | |
| | | | | [Sorghum | |
| | | | | bicolor] | |
| 1077 | 1005 | Zea mays | 0.897009967 | nuclear | 195634708 |
| | | | | transcription | |
| | | | | factor Y | |
| | | | | subunit A-3 | |
| | | | | [Zea mays] | |
| 1078 | 1006 | Zea mays | 0.890365449 | hypothetical | 212723473 |
| | | | | protein | |
| | | | | LOC100194182 | |
| | | | | [Zea | |
| | | | | mays] | |
| | | | | &gt;gi\|194695138\| | |
| | | | | gb\|ACF81653.1\| | |
| | | | | unknown | |
| | | | | [Zea mays] | |
| | | | | &gt;gi\|195625280\| | |
| | | | | gb\|ACG34470.1\| | |
| | | | | nuclear | |
| | | | | transcription | |
| | | | | factor Y | |
| | | | | subunit A-3 | |
| | | | | [Zea mays] | |
| 1079 | 1007 | Zea mays | 0.887043189 | unknown | 224028448 |
| | | | | [Zea mays] | |
| 1080 | 1008 | Zea mays | 0.853820598 | unknown | 194699259 |
| | | | | [Zea mays] | |
| 1081 | 1009 | Zea mays | 0.853820598 | nuclear | 195609807 |
| | | | | transcription | |
| | | | | factor Y | |
| | | | | subunit A-3 | |
| | | | | [Zea mays] | |
| 1082 | 1010 | Zea mays | 0.850498339 | nuclear | 226499901 |
| | | | | transcription | |
| | | | | factor Y | |
| | | | | subunit A-3 | |
| | | | | [Zea mays] | |
| | | | | &gt;gi\|195609780\| | |
| | | | | gb\|ACG26720.1\| | |
| | | | | nuclear | |
| | | | | transcription | |
| | | | | factor Y | |
| | | | | subunit A-3 | |
| | | | | [Zea mays] | |
| 1083 | 1011 | Zea mays | 1 | hypothetical | 212723473 |
| | | | | protein | |
| | | | | LOC100194182 | |
| | | | | [Zea | |
| | | | | mays] | |
| | | | | &gt;gi\|194695138\| | |
| | | | | gb\|ACF81653.1\| | |
| | | | | unknown | |
| | | | | [Zea mays] | |
| | | | | &gt;gi\|195625280\| | |
| | | | | gb\|ACG34470.1\| | |
| | | | | nuclear | |
| | | | | transcription | |
| | | | | factor Y | |
| | | | | subunit A-3 | |
| | | | | [Zea mays] | |
| 1084 | 1012 | Zea mays | 0.996666667 | unknown | 224028448 |
| | | | | [Zea mays] | |
| 1085 | 1013 | Zea mays | 0.98 | nuclear | 195634708 |
| | | | | transcription | |
| | | | | factor Y | |
| | | | | subunit A-3 | |
| | | | | [Zea mays] | |
| | 1014 | Sorghum | 0.893333333 | hypothetical | 255761094 |
| | | bicolor | | protein | |
| | | | | SORBIDRAFT_02g038960 | |
| | | | | [Sorghum | |
| | | | | bicolor] | |
| | | | | &gt;gi\|241926544\| | |
| | | | | gb\|EER99688.1\| | |
| | | | | hypothetical | |
| | | | | protein | |
| | | | | SORBIDRAFT_02g038960 | |
| | | | | [Sorghum | |
| | | | | bicolor] | |
| 1086 | 1015 | Zea mays | 0.853333333 | unknown | 194699259 |
| | | | | [Zea mays] | |
| 1087 | 1016 | Zea mays | 0.856666667 | nuclear | 195609807 |
| | | | | transcription | |
| | | | | factor Y | |
| | | | | subunit A-3 | |
| | | | | [Zea mays] | |
| 1088 | 1017 | Zea mays | 0.853333333 | nuclear | 226499901 |
| | | | | transcription | |
| | | | | factor Y | |
| | | | | subunit A-3 | |
| | | | | [Zea mays] | |
| | | | | &gt;gi\|195609780\| | |
| | | | | gb\|ACG26720.1\| | |
| | | | | nuclear | |
| | | | | transcription | |
| | | | | factor Y | |
| | | | | subunit A-3 | |
| | | | | [Zea mays] | |
| 1089 | 1018 | Zea mays | 1 | nuclear | 226502984 |
| | | | | transcription | |
| | | | | factor Y | |
| | | | | subunit A-3 | |
| | | | | [Zea mays] | |
| | | | | &gt;gi\|195624530\| | |
| | | | | gb\|ACG34095.1\| | |
| | | | | nuclear | |
| | | | | transcription | |
| | | | | factor Y | |
| | | | | subunit A-3 | |
| | | | | [Zea mays] | |
| | 1019 | Sorghum | 0.814545455 | hypothetical | 255761094 |
| | | bicolor | | protein | |
| | | | | SORBIDRAFT_04g034760 | |
| | | | | [Sorghum | |
| | | | | bicolor] | |
| | | | | &gt;gi\|241934478\| | |
| | | | | gb\|EES07623.1\| | |
| | | | | hypothetical | |
| | | | | protein | |
| | | | | SORBIDRAFT_04g034760 | |
| | | | | [Sorghum | |
| | | | | bicolor] | |
| | 1020 | Sorghum | 1 | hypothetical | 255761094 |
| | | bicolor | | protein | |
| | | | | SORBIDRAFT_01g004290 | |
| | | | | [Sorghum | |
| | | | | bicolor] | |
| | | | | &gt;gi\|241917544\| | |
| | | | | gb\|EER90688.1\| | |
| | | | | hypothetical | |
| | | | | protein | |
| | | | | SORBIDRAFT_01g004290 | |
| | | | | [Sorghum | |
| | | | | bicolor] | |
| 1090 | 1021 | Zea mays | 0.836633663 | unknown | 194696171 |
| | | | | [Zea mays] | |

| | Protein | Homologue | miR | | |
|---|---|---|---|---|---|
| Nucleotide | seq id | NCBI | Binding | miR | miR |
| seq id no: | no: | Accession | Position | sequence | name |
| | 927 | XP_002463896 | 426-446 | GTGAAG | zma- |
| | | | | TGTTTG | miR395b |
| | | | | GGGGAA | |
| | | | | CTC/4 | |
| 1022 | 928 | ACN28609 | | | |
| 1023 | 929 | ACN34023 | | | |
| 1024 | 930 | ACG45192 | | | |
| 1025 | 931 | NP_001104877 | | | |
| | 932 | EAY91825 | | | |
| 1026 | 933 | NP_001051234 | | | |
| 1027 | 934 | BAK05662 | | | |
| 1028 | 935 | BAA36274 | | | |
| | 936 | EAZ28548 | | | |
| | 937 | XP_002441904 | 352-372 | | |
| 1029 | 938 | NP_001066285 | | | |
| | 939 | EEE52851 | | | |
| | 940 | EEC68940 | | | |
| 1030 | 941 | ACN34023 | 616-636 | | |
| 1031 | 942 | NP_001104877 | | | |
| 1032 | 943 | ACN28609 | | | |
| 1033 | 944 | ACG45192 | | | |
| | 945 | XP_002463896 | | | |
| 1034 | 946 | BAK05662 | | | |
| | 947 | EAY91825 | | | |
| 1035 | 948 | NP_001051234 | | | |
| 1036 | 949 | BAA36274 | | | |
| | 950 | EAZ28548 | | | |
| | 951 | XP_002452486 | 1000-1020 | GGAATC | zma- |
| | | | | TTGATG | miR172e |
| | | | | ATGCTG | |
| | | | | CAT/3 | |
| 1037 | 952 | ACN31224 | | | |
| 1038 | 953 | NP_001143596 | | | |
| 1039 | 954 | NP_001144184 | | | |
| 1040 | 955 | NP_001047497 | | | |
| 1041 | 956 | BAJ96123 | | | |
| 1042 | 957 | ABR19871 | 869-889 | | |
| 1043 | 958 | ABR19870 | | | |
| | 959 | XP_002459580 | 1539-1559 | | |
| 1044 | 960 | NP_001139539 | | | |
| 1045 | 961 | ACN58224 | | | |
| 1046 | 962 | ACG46304 | | | |
| 1047 | 963 | NP_001044720 | 1121-1141 | | |
| 1048 | 964 | NP_001131287 | | | |
| 1049 | 965 | ACG32843 | | | |
| 1050 | 966 | ACF06518 | | | |
| 1051 | 967 | ACF06519 | | | |
| 1052 | 968 | ABB16993 | | | |
| | 969 | XP_002523902 | | | |
| 1053 | 970 | XP_002279025 | | | |
| 1054 | 971 | ACN30570 | 882-902 | AGAAGA | zma- |
| | | | | GAGAGA | miR529 |
| | | | | GTACAG | |
| | | | | CCT/1 | |
| 1055 | 972 | NP_001145445 | | | |
| | 973 | XP_002450775 | | | |
| | 974 | XP_002455799 | 45-65 | | |
| 1056 | 975 | ACN27525 | | | |
| 1057 | 976 | NP_001145223 | | | |
| 1058 | 977 | ACF86782 | 9910, 6-916 | | |
| 1059 | 978 | ACG45113 | | | |
| | 979 | XP_002462571 | | | |
| 1060 | 980 | NP_001136945 | | | |
| 1061 | 981 | NP_001149534 | 1348-1368 | | |
| | 982 | XP_002438971 | | | |
| 1062 | 983 | NP_001136945 | 973-993 | | |
| | 984 | XP_002462571 | | | |
| 1063 | 985 | ACF86782 | | | |
| 1064 | 986 | ACG45113 | | | |
| 1065 | 987 | CAB56631 | 558-578 | | |
| | 988 | XP_002444771 | | | |
| 1066 | 989 | ACL52941 | | | |
| 1067 | 990 | NP_001152658 | 1410-1430 | | |
| 1068 | 991 | ACF83838 | | | |
| 1069 | 992 | ACG39594 | | | |
| | 993 | XP_002445813 | | | |
| 1070 | 994 | ACN31483 | | | |
| 1071 | 995 | NP_001056592 | | | |
| 1072 | 996 | AAL58883 | | | |
| 1073 | 997 | ABR25495 | | | |
| 1074 | 998 | BAK03317 | | | |
| | 999 | EAY99382 | | | |
| 1075 | 1000 | AAX83872 | 1197-1217 | | |
| | 1001 | AAX83875 | | | |
| 1076 | 1002 | AAX83873 | | | |
| | 1003 | XP_002445815 | | | |
| | 1004 | XP_002463167 | 1112-1132 | TAGCCA | zma- |
| | | | | GGGATG | miR1691 |
| | | | | ATTTGC | |
| | | | | CTG/2 | |
| 1077 | 1005 | ACG36823 | | | |
| 1078 | 1006 | NP_001132701 | | | |
| 1079 | 1007 | ACN33300 | | | |
| 1080 | 1008 | ACF83714 | | | |
| 1081 | 1009 | ACG26734 | | | |
| 1082 | 1010 | NP_001147311 | | | |
| 1083 | 1011 | NP_001132701 | 1108-1128 | | |
| 1084 | 1012 | ACN33300 | | | |
| 1085 | 1013 | ACG36823 | | | |
| | 1014 | XP_002463167 | | | |
| 1086 | 1015 | ACF83714 | | | |
| 1087 | 1016 | ACG26734 | | | |
| 1088 | 1017 | NP_001147311 | | | |
| 1089 | 1018 | NP_001149075 | 979-999 | | |
| | 1019 | XP_002454647 | | | |
| | 1020 | XP_002463690 | 946-966 | | |
| 1090 | 1021 | ACF82170 | | | |

### Example 4

### Verification of Expression of miRNAs Associated with Increased NUE

Following identification of dsRNAs potentially involved in improvement of maize NUE using bioinformatics tools, as described in Examples 1-2 above, the actual mRNA levels were determined using reverse transcription assay followed by quantitative Real-Time PCR (qRT-PCR) analysis. RNA levels were compared between different tissues, developmental stages, growth conditions and/or genetic backgrounds incorporated. A correlation analysis between mRNA levels in different experimental conditions/genetic backgrounds was applied and used as evidence for the role of the gene in the plant.

Methods

Mobile nutrients such as N reach their targets and are then recycled, often executed in the form of simultaneous import and export of the nutrients from leaves. This dynamic nutrient cycling is termed remobilization or retranslocation, and thus leaf analyses are highly recommended. For that reason, root and leaf samples were freshly excised from maize plants grown as described above on Murashige-Skoog without Ammonium Nitrate (NH₄NO₃) (Duchefa). Experimental plants were grown either under optimal ammonium nitrate concentrations (100%) and used as a control group, or under stressful conditions of 10% or 1% ammonium nitrate used as stress-induced groups. Total RNA was extracted from the different tissues, using mirVana™ commercial kit (Ambion) following the protocol provided by the manufacturer. For measurement and verification of messenger RNA (mRNA) expression level of all genes, reverse transcription followed by quantitative real time PCR (qRT-PCR) was performed on total RNA extracted from each plant tissue (i.e., roots and leaves) from each experimental group as described above. To elaborate, reverse transcription was performed on 1 μg total RNA, using a miScript Reverse Transcriptase kit (Qiagen), following the protocol suggested by the manufacturer. Quantitative RT-PCR was performed on cDNA (0.1 ng/μl final concentration), using a miScript SYBR GREEN PCR (Qiagen) forward (based on the miR sequence itself) and reverse primers (supplied with the kit). All qRT-PCR reactions were performed in triplicates using an ABI7500 real-time PCR machine, following the recommended protocol for the machine. To normalize, the expression level of miRNAs associated with enhanced NUE between the different tissues and growth conditions of the maize plants, normalizer miRNAs were used for comparison. Normalizer miRNAs, which are miRNAs with unchanged expression level between tissues and growth conditions, were custom selected for each experiment. The normalization procedure consisted of second-degree polynomial fitting to a reference data (which is the median vector of all the data—excluding outliers) as described by Rosenfeld et al (2008, Nat Biotechnol, 26(4):462-469). A summary of primers for the differential miRNAs that was used in the qRT-PCR analysis is presented in Table 7a below. The results of the qRT-PCR analyses under different nitrogen concentrations (1% and 10% versus optimal 100%) are presented in Tables 7b-d below.

TABLE 7a
Primers of Small RNAs used for qRT-PCR Validation Analysis.

| Primer Length | Primer Sequence/SEQ ID NO: | Small RNA Name |
|---|---|---|
| 24 | GGCAGAAGAGAGAGAGTACAGCCT/1091 | Zma-miR529 |
| 23 | GCTAGCCAGGGATGATTTGCCTG/1092 | Zma-miR169l |
| 21 | AGGATGCTGACGCAATGGGAT/1093 | Predicted zma mir 48486 |
| 25 | TGGCTTAGATGACCATCAGCAAACA/1094 | Zma-miR827 |
| 23 | GCGTGAAGTGTTTGGGGGAACTC/1095 | Zma-miR395b |
| 22 | CTAGCCAAGCATGATTTGCCCG/1096 | Predicted zma mir 50601 |
| 23 | CAGGATGTGAGGCTATTGGGGAC/1097 | Predicted zma mir 48492 |
| 22 | CCAAGTCGAGGGCAGACCAGGC/1098 | Predicted zma mir 48879 |
| 21 | ATTCACGGGGACGAACCTCCT/1099 | Mtr-miR2647a |
| 24 | GGCGGAATCTTGATGATGCTGCAT/1100 | Zma-miR172e |

TABLE 7b
Results of qRT-PCR Validation Analysis on Differential Small RNAs-
1% Nitrogen vs. Control (100% Nitrogen).

| | Fold | | | |
|---|---|---|---|---|
| p-value | Change | Direction | Sequence/SEQ ID NO: | miR Name |
| 3.20E−03 | 1.68 | up | TTAGATGACCATCAGCAAACA/10 | zma-miR827 |
| 3.60E−03 | 1.96 | up | CCAAGTCGAGGGCAGACCAGGC/7 | Predicted zma mir 48879 |
| 4.40E−02 | 1.55 | up | AGGATGCTGACGCAATGGGAT/9 | Predicted zma mir 48486 |
| 1.30E−03 | −3.16 | down | GTGAAGTGTTTGGGGGAACTC/4 | zma-miR395b |

TABLE 7c
Results of qRT-PCR Validation Analysis on Differential Small RNAs-
1% Nitrogen vs. 10% Nitrogen.

| | Fold | | | |
|---|---|---|---|---|
| p-value | Change | Direction | Sequence/SEQ ID NO: | miR Name |
| 2.30E−02 | 2.42 | up | AGGATGCTGACGCAATGGGAT/9 | Predicted zma mir 48486 |
| 1.30E−02 | 1.62 | up | TTAGATGACCATCAGCAAACA/10 | zma-miR827 |
| 4.60E−02 | 1.57 | up | AGGATGTGAGGCTATTGGGGAC/6 | Predicted zma mir 48492 |

TABLE 7d
Results of qRT-PCR Validation Analysis on Differential Small RNAs-
10% Nitrogen vs. Control (100% Nitrogen).

| | Fold | | | |
|---|---|---|---|---|
| p-value | Change | Direction | Sequence/SEQ ID NO: | miR Name |
| 4.50E−03 | −3.71 | down | GTGAAGTGTTTGGGGGAACTC/4 | zma-miR395b |

### Example 5

### Gene Cloning and Creation of Binary Vectors for Plant Expression

Cloning Strategy—the best validated miRNAs are cloned into pORE-E1 binary vectors for the generation of transgenic plants. The full-length open reading frame (ORF) comprising of the hairpin sequence of each selected miRNA, is synthesized by Genscript (Israel). The resulting clone is digested with appropriate restriction enzymes and inserted into the Multi Cloning Site (MCS) of a similarly digested binary vector through ligation using T4 DNA ligase enzyme (Promega, Madison, Wis., USA).

### Example 6

### Generation of Transgenic Model Plants Expressing the NUE Small RNAs

Arabidoposis thaliana transformation is performed using the floral dip procedure following a slightly modified version of the published protocol (ref). Briefly, TO Plants are planted in small pots filled with soil. The pots are covered with aluminum foil and a plastic dome, kept at 4° C. for 3-4 days, then uncovered and incubated in a growth chamber at 24° C. under 16 hr light:8 hr dark cycles. A week prior to transformation all individual flowering stems are removed to allow for growth of multiple flowering stems instead. A single colony of Agrobacterium (GV3101) carrying the binary vectors (pORE-E1), harboring the NUE miRNA hairpin sequences with additional flanking sequences both upstream and downstream of it, is cultured in LB medium supplemented with kanamycin (50 mg/L) and gentamycin (25 mg/L). Three days prior to transformation, each culture is incubated at 28° C. for 48 hrs, shaking at 180 rpm. The starter culture is split the day before transformation into two cultures, which are allowed to grow further at 28° C. for 24 hours at 180 rpm. Pellets containing the agrobacterium cells are obtained by centrifugation of the cultures at 5000 rpm for 15 minutes. The pellets are resuspended in an infiltration medium (10 mM MgCl₂, 5% sucrose, 0.044 μM BAP (Sigma) and 0.03% Tween 20) in double-distilled water.

Transformation of T0 plants is performed by inverting each plant into the Agrobacterium suspension, keeping the flowering stem submerged for 5 minutes. Following inoculation, each plant is blotted dry for 5 minutes on both sides, and placed sideways on a fresh covered tray for 24 hours at 22° C. Transformed (transgenic) plants are then uncovered and transferred to a greenhouse for recovery and maturation. The transgenic T0 plants are grown in the greenhouse for 3-5 weeks until the seeds are ready, which are then harvested from plants and kept at room temperature until sowing.

### Example 7

### Selection of Transgenic Arabidopsis Plants Expressing the NUE Genes According to Expression Level

Arabidopsis seeds are sown and sprayed with Basta (Bayer) on 1-2 weeks old seedlings, at least twice every few days. Only resistant plants, which are heterozygous for the transgene, survive. PCR on the genomic gene sequence is performed on the surviving seedlings using primers pORE-F2 (fwd, 5′-TTTAGCGATGAACTTCACTC-3′, SEQ ID NO: 20) and a custom designed reverse primer based on each miR's sequence.

### Example 8

### Evaluating Changes in Root Architecture in Transgenic Plants

Many key traits in modern agriculture can be explained by changes in the root architecture of the plant. Root size and depth have been shown to logically correlate with drought tolerance, since deeper root systems can access water stored in deeper soil layers. Correspondingly, a highly branched root system provides better coverage of the soil and therefore can effectively absorb all micro and macronutrients available, resulting in enhanced NUE.

To test whether the transgenic plants produce a modified root structure, plants can be grown in agar plates placed vertically. A digital picture of the plates is taken every few days and the maximal length and total area covered by the plant roots are assessed. From every construct created, several independent transformation events are checked in replicates. To assess significant differences between root features, a statistical test, such as a Student's t-test, is employed in order to identify enhanced root features and to provide a statistical value to the findings.

### Example 9

### Testing for Increased Nitrogen Use Efficiency (NUE)

To analyze whether the transgenic Arabidopsis plants are more responsive to nitrogen, plants are grown in two different nitrogen concentrations: (1) optimal nitrogen concentration (100% NH₄NO₃, which corresponds to 20.61 mM) or (2) nitrogen deficient conditions (1% or 10% NH₄NO₃, which corresponds to 0.2 and 2.06 mM, respectively). Plants are allowed to grow until seed production followed by an analysis of their overall size, time to flowering, yield, protein content of shoot and/or grain, and seed production. The parameters checked can be the overall size of the plant, wet and dry weight, the weight of the seeds yielded, the average seed size and the number of seeds produced per plant. Other parameters that may be tested are: the chlorophyll content of leaves (as nitrogen plant status and the degree of leaf greenness are highly correlated), amino acid and the total protein content of the seeds or other plant parts such as leaves or shoots and oil content. Transformed plants not exhibiting substantial physiological and/or morphological effects, or exhibiting higher measured parameters levels than wild-type plants, are identified as nitrogen use efficient plants.

### Example 10

### Method for Generating Transgenic Maize Plants with Enhanced or Reduced microRNA Regulation of Target Genes

Target prediction enables two contrasting strategies; an enhancement (positive) or a reduction (negative) of microRNA regulation. Both these strategies have been used in plants and have resulted in significant phenotype alterations. For complete in-vivo assessment of the phenotypic effects of the differential miRNAs in this invention, the inventors implement both over-expression and down-regulation methods on all miRNAs found to associate with NUE as listed in Table 1. Reduction of miRNA regulation of target genes can be accomplished in one of two approaches:

### Expressing a microRNA-Resistant Target

In this method, silent mutations are introduced in the microRNA binding site of the target gene so that the DNA and resulting RNA sequences are changed to prevent microRNA binding, but the amino acid sequence of the protein is unchanged.

### Expressing a Target-Mimic Sequence

Plant microRNAs usually lead to cleavage of their targeted gene, with this cleavage typically occurring between bases 10 and 11 of the microRNA. This position is therefore especially sensitive to mismatches between the microRNA and the target. It was found that expressing a DNA sequence that could potentially be targeted by a microRNA, but contains three extra nucleotides (ATC) between the two nucleotides that are predicted to hybridize with bases 10-11 of the microRNA (thus creating a bulge in that position), can inhibit the regulation of that microRNA on its native targets (Franco-Zorilla J M et al., Nat Genet 2007; 39(8):1033-1037).

This type of sequence is referred to as a “target-mimic”. Inhibition of the microRNA regulation is presumed to occur through physically capturing the microRNA by the target-mimic sequence and titering-out the microRNA, thereby reducing its abundance. This method was used to reduce the amount and, consequentially, the regulation of microRNA 399 in Arabidopsis.

TABLE 8
miRNA-Resistant Target Examples for Selected down-regulated miRNAs of
the Invention.

| | Mutated | ORF | Original | | | | | | |
|---|---|---|---|---|---|---|---|---|---|
| NCBI | Nucleo- | Nucleo- | Nucleo- | | | | | MiR | |
| MiR | tide | tide | tide | Protein | | Homolog | | sequence/ | |
| Binding | SEQ ID | SEQ ID | SEQ ID | SEQ ID | | NCBI | WMD3 | SEQ ID | MiR |
| Site | NO: | NO: | NO: | NO: | Organism | Accession | Targets | NO: | name |
| | | | | | | | miR | TTAGAT | zma- |
| | | | | | | | binding | GACCAT | miR827 |
| | | | | | | | site: | CAGCAA | |
| | | | | | | | TC372606 | ACA/10 | |
| | | | | | | | -> not | | |
| | | | | | | | found on | | |
| | | | | | | | the master | | |
| | | | | | | | seq | | |
| | | 1103 | 1102 | 1101 | Zea mays | NP_ | TC372597 | | |
| | | | | | | 001105530 | | | |
| 1825- | 1104 | | | | | | | | |
| 1845 | | | | | | | | | |
| 1825- | 1105 | | | | | | | | |
| 1845 | | | | | | | | | |
| 1825- | 1106 | | | | | | | | |
| 1845 | | | | | | | | | |
| 1825- | 1107 | | | | | | | | |
| 1845 | | | | | | | | | |
| 1825- | 1108 | | | | | | | | |
| 1845 | | | | | | | | | |
| 1825- | 1109 | | | | | | | | |
| 1845 | | | | | | | | | |
| 1825- | 1110 | | | | | | | | |
| 1845 | | | | | | | | | |
| 1825- | 1111 | | | | | | | | |
| 1845 | | | | | | | | | |
| 1825- | 1112 | | | | | | | | |
| 1845 | | | | | | | | | |
| 1825- | 1113 | | | | | | | | |
| 1845 | | | | | | | | | |
| | | 1116 | 1115 | 1114 | Zea mays | NP_ | GRMZM2 | | |
| | | | | | | 001130294 | G013176_ | | |
| | | | | | | | T02 | | |
| 1017- | 1117 | | | | | | | | |
| 1037 | | | | | | | | | |
| 1017- | 1118 | | | | | | | | |
| 1037 | | | | | | | | | |
| 1017- | 1119 | | | | | | | | |
| 1037 | | | | | | | | | |
| 1017- | 1120 | | | | | | | | |
| 1037 | | | | | | | | | |
| 1017- | 1121 | | | | | | | | |
| 1037 | | | | | | | | | |
| 1017- | 1122 | | | | | | | | |
| 1037 | | | | | | | | | |
| 1017- | 1123 | | | | | | | | |
| 1037 | | | | | | | | | |
| 1017- | 1124 | | | | | | | | |
| 1037 | | | | | | | | | |
| | | | | | | | target: | AGGATG | Predicted |
| | | | | | | | TC422488 | CTGACG | zma mir |
| | | | | | | | of Mir | CAATGG | 48486 |
| | | | | | | | Predicted | GAT/9 | |
| | | | | | | | zma mir | | |
| | | | | | | | 48486 is | | |
| | | | | | | | located in | | |
| | | | | | | | UTR | | |

TABLE 9
miRNA-Resistant Target Examples for Selected up-regulated miRNAs of
the Invention.

| | Mutated | ORF | Original | | | | | | |
|---|---|---|---|---|---|---|---|---|---|
| NCBI | Nucleo- | Nucleo- | Nucleo- | | | | | | |
| Mir | tide | tide | tide | Protein | | Homolog | | | |
| Binding | SEQ ID | SEQ ID | SEQ ID | SEQ ID | | NCBI | WMD3 | MiR | |
| Site | NO: | NO: | NO: | NO: | Organism | Accession | Targets | sequence | MiR name |
| | | 1127 | 1126 | 1125 | Zea mays | ACN34023 | GRMZM2 | GTGAA | zma- |
| | | | | | | | G051270_ | GTGTTT | miR395b |
| | | | | | | | T01 | GGGGG | |
| | | | | | | | | AACTC/4 | |
| 527- | 1128 | | | | | | | | |
| 547 | | | | | | | | | |
| 527- | 1129 | | | | | | | | |
| 547 | | | | | | | | | |
| 527- | 1130 | | | | | | | | |
| 547 | | | | | | | | | |
| 527- | 1131 | | | | | | | | |
| 547 | | | | | | | | | |
| 527- | 1132 | | | | | | | | |
| 547 | | | | | | | | | |
| 527- | 1133 | | | | | | | | |
| 547 | | | | | | | | | |
| 527- | 1134 | | | | | | | | |
| 547 | | | | | | | | | |
| | | 1137 | 1136 | 1135 | Zea mays | ACN30570 | TC441933 | AGAAG | zma- |
| | | | | | | | | AGAGA | miR529 |
| | | | | | | | | GAGTAC | |
| | | | | | | | | AGCCT/1 | |
| 889- | 1138 | | | | | | | | |
| 909 | | | | | | | | | |
| 889- | 1139 | | | | | | | | |
| 909 | | | | | | | | | |
| 889- | 1140 | | | | | | | | |
| 909 | | | | | | | | | |
| 889- | 1141 | | | | | | | | |
| 909 | | | | | | | | | |
| 889- | 1142 | | | | | | | | |
| 909 | | | | | | | | | |
| 889- | 1143 | | | | | | | | |
| 909 | | | | | | | | | |
| 889- | 1144 | | | | | | | | |
| 909 | | | | | | | | | |
| 889- | 1145 | | | | | | | | |
| 909 | | | | | | | | | |
| 889- | 1146 | | | | | | | | |
| 909 | | | | | | | | | |
| 889- | 1147 | | | | | | | | |
| 909 | | | | | | | | | |
| | | 1150 | 1149 | 1148 | Zea mays | ACF86782 | TC374118 | | |
| 923- | 1151 | | | | | | | | |
| 943 | | | | | | | | | |
| 923- | 1152 | | | | | | | | |
| 943 | | | | | | | | | |
| 923- | 1153 | | | | | | | | |
| 943 | | | | | | | | | |
| 923- | 1154 | | | | | | | | |
| 943 | | | | | | | | | |
| 923- | 1155 | | | | | | | | |
| 943 | | | | | | | | | |
| 923- | 1156 | | | | | | | | |
| 943 | | | | | | | | | |
| 923- | 1157 | | | | | | | | |
| 943 | | | | | | | | | |
| 923- | 1158 | | | | | | | | |
| 943 | | | | | | | | | |
| 923- | 1159 | | | | | | | | |
| 943 | | | | | | | | | |
| 923- | 1160 | | | | | | | | |
| 943 | | | | | | | | | |
| | | 1163 | 1162 | 1161 | Zea mays | NP_ | GRMZM2 | | |
| | | | | | | 001149534 | G414805_ | | |
| | | | | | | | T04 | | |
| 1396- | 1164 | | | | | | | | |
| 1416 | | | | | | | | | |
| 1396- | 1165 | | | | | | | | |
| 1416 | | | | | | | | | |
| 1396- | 1166 | | | | | | | | |
| 1416 | | | | | | | | | |
| 1396- | 1167 | | | | | | | | |
| 1416 | | | | | | | | | |
| 1396- | 1168 | | | | | | | | |
| 1416 | | | | | | | | | |
| 1396- | 1169 | | | | | | | | |
| 1416 | | | | | | | | | |
| 1396- | 1170 | | | | | | | | |
| 1416 | | | | | | | | | |
| 1396- | 1171 | | | | | | | | |
| 1416 | | | | | | | | | |
| 1396- | 1172 | | | | | | | | |
| 1416 | | | | | | | | | |
| 1396- | 1173 | | | | | | | | |
| 1416 | | | | | | | | | |
| | | 1176 | 1175 | 1174 | Zea mays | NP_ | GRMZM2 | | |
| | | | | | | 001136945 | G126018_ | | |
| | | | | | | | T01 | | |
| 926- | 1177 | | | | | | | | |
| 946 | | | | | | | | | |
| 926- | 1178 | | | | | | | | |
| 946 | | | | | | | | | |
| 926- | 1179 | | | | | | | | |
| 946 | | | | | | | | | |
| 926- | 1180 | | | | | | | | |
| 946 | | | | | | | | | |
| 926- | 1181 | | | | | | | | |
| 946 | | | | | | | | | |
| 926- | 1182 | | | | | | | | |
| 946 | | | | | | | | | |
| 926- | 1183 | | | | | | | | |
| 946 | | | | | | | | | |
| 926- | 1184 | | | | | | | | |
| 946 | | | | | | | | | |
| 926- | 1185 | | | | | | | | |
| 946 | | | | | | | | | |
| 926- | 1186 | | | | | | | | |
| 946 | | | | | | | | | |
| | | 1189 | 1188 | 1187 | Zea mays | CAB56631 | GRMZM2 | | |
| | | | | | | | G160917_ | | |
| | | | | | | | T01 | | |
| 589- | 1190 | | | | | | | | |
| 609 | | | | | | | | | |
| 589- | 1191 | | | | | | | | |
| 609 | | | | | | | | | |
| 589- | 1192 | | | | | | | | |
| 609 | | | | | | | | | |
| 589- | 1193 | | | | | | | | |
| 609 | | | | | | | | | |
| 589- | 1194 | | | | | | | | |
| 609 | | | | | | | | | |
| 589- | 1195 | | | | | | | | |
| 609 | | | | | | | | | |
| 589- | 1196 | | | | | | | | |
| 609 | | | | | | | | | |
| 589- | 1197 | | | | | | | | |
| 609 | | | | | | | | | |
| 589- | 1198 | | | | | | | | |
| 609 | | | | | | | | | |
| 589- | 1199 | | | | | | | | |
| 609 | | | | | | | | | |
| | | | | | | | | target: | |
| | | | | | | | | GRMZM2 | |
| | | | | | | | | G101511_ | |
| | | | | | | | | T01 of | |
| | | | | | | | | Mir zma- | |
| | | | | | | | | miR529 is | |
| | | | | | | | | located in | |
| | | | | | | | | UTR | |
| | | | | | | | target: | TAGCCA | zma- |
| | | | | | | | TC374958 | GGGAT | miR169l |
| | | | | | | | of Mir | GATTTG | |
| | | | | | | | zma- | CCTG/2 | |
| | | | | | | | miR169l | | |
| | | | | | | | is located | | |
| | | | | | | | in UTR | | |
| | | | | | | | | target: | |
| | | | | | | | | TC391807 | |
| | | | | | | | | of Mir | |
| | | | | | | | | zma- | |
| | | | | | | | | miR169l | |
| | | | | | | | | is located | |
| | | | | | | | | in UTR | |

TABLE 10
Target Mimic Examples for Selected up-regulated miRNAs of the
Invention

| | | Bulge | | |
|---|---|---|---|---|
| | Bulge in Target | Reverse | | |
| Full Target Mimic | Binding | Complement | MiR | |
| Nucleotide | Sequence/SEQ | miR/SEQ | sequence/SEQ | MiR |
| Seq/SEQ ID NO: | ID NO: | ID NO: | ID NO: | name |
| 1208 | GAGTTCCTCC | GAGTTCCC | GTGAAGT | zma- |
| | ACTAAGGCAC | CCACTAAA | GTTTGGG | miR395b |
| | TTCAT/1204 | CACTTCAC/1200 | GGAACTC/4 | |
| 11 | CTGCAGCAT | ATGCAGCA | GGAATCT | zma- |
| | CACTATCAG | TCACTATCA | TGATGAT | miR172e |
| | GATTCT/1205 | AGATTCC/1201 | GCTGCAT/3 | |
| 12 | CGAGTGTGC | AGGCTGTA | AGAAGA | zma- |
| | TCCTATCTCT | CTCCTATCT | GAGAGA | miR529 |
| | CTTCT/1206 | CTCTTCT/1202 | GTACAGCCT/1 | |
| 13 | GTGGCAACT | CAGGCAAA | TAGCCAG | zma- |
| | CACTATCCTT | TCACTATCC | GGATGAT | miR169l |
| | GGCTC/1207 | CTGGCTA/1203 | TTGCCTG/2 | |

TABLE 11
Target Mimic Examples for Selected up-regulated miRNAs of the
Invention

| | Bulge in | Bulge | | |
|---|---|---|---|---|
| | Target | Reverse | | |
| Full Target Mimic | Binding | Complement | MiR | MiR |
| Nucleotide Seq | Sequence | miR | sequence | name |
| 18 | TGTTAG | TGTTTGCTG | TTAGATG | zma- |
| | CTGATC | ATCTAGGT | ACCATCA | miR827 |
| | TAGGTC | CATCTAA/14 | GCAAACA/10 | |
| | ATATAC/16 | | | |
| 19 | TTCCCCC | ATCCCATTG | AGGATGC | Predicted |
| | TGCGCT | CGCTATCA | TGACGCA | zma |
| | ATCAGC | GCATCCT/15 | ATGGGAT/9 | mir |
| | TTCCT/17 | | | 48486 |

TABLE 12
Abbreviations of plant species

| | | Abbre- |
|---|---|---|
| Common Name | Organism Name | viation |
| Peanut | Arachis hypogaea | ahy |
| Arabidopsis lyrata | Arabidopsis lyrata | aly |
| Rocky Mountain Columbine | Aquilegia coerulea | aqc |
| Tausch's goatgrass | Aegilops taushii | ata |
| Arabidopsis thaliana | Arabidopsis thaliana | ath |
| Grass | Brachypodium distachyon | bdi |
| Brassica napus canola (“liftit”) | Brassica napus | bna |
| Brassica oleracea wild cabbage | Brassica oleracea | bol |
| Brassica rapa yellow mustard | Brassica rapa | bra |
| Clementine | Citrus Clementine | ccl |
| Orange | Citrus sinensis | csi |
| Trifoliate orange | Citrus trifoliata | ctr |
| Glycine max | Glycine max | gma |
| Wild soybean | Glycine soja | gso |
| Barley | Hordeum vulgare | hvu |
| Lotus japonicus | Lotus japonicus | lja |
| Medicago truncatula - Barrel | Medicago truncatula | mtr |
| Clover (“tiltan”) | | |
| Oryza sativa | Oryza sativa | osa |
| European spruce | Picea abies | pab |
| Physcomitrella patens (moss) | Physcomitrella patens | ppt |
| Pinus taeda - Loblolly Pine | Pinus taeda | pta |
| Populus trichocarpa - black | Populus trichocarpa | ptc |
| cotton wood | | |
| Castor bean (“kikayon”) | Ricinus communis | rco |
| Sorghum bicolor Dura | Sorghum bicolor | sbi |
| tomato microtom | Solanum lycopersicum | sly |
| Selaginella moellendorffii | Selaginella moellendorffii | smo |
| Sugarcane | Saccharum officinarum | sof |
| Sugarcane | Saccharum spp | ssp |
| Triticum aestivum | Triticum aestivum | tae |
| cacao tree and cocoa tree | Theobroma cacao | tcc |
| Vitis vinifera Grapes | Vitis vinifera | vvi |
| corn | Zea mays | zma |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of improving nitrogen use efficiency, abiotic stress tolerance, biomass, vigor or yield of a plant, the method comprising expressing within the plant an exogenous polynucleotide having a nucleic acid sequence at least 90% identical to SEQ ID NOs: 10, 6-9, 21, 22, 23-37, 38-52, 1209, 1211, 1212, wherein said nucleic acid sequence is capable of regulating nitrogen use efficiency of the plant, thereby improving nitrogen use efficiency, abiotic stress tolerance, biomass, vigor or yield of the plant.

2. A transgenic plant exogenously expressing a polynucleotide having a nucleic acid sequence at least 90% identical to SEQ ID NOs: 10, 6-9, 23-37, wherein said nucleic acid sequence is capable of regulating nitrogen use efficiency of the plant.

3. The method of claim 1, wherein said exogenous polynucleotide encodes a precursor of said nucleic acid sequence.

4. The method of claim 3, wherein said precursor of said nucleic acid sequence is at least 60% identical to SEQ ID NO: 21, 22, 38-52, 1209, 1211, 1212.

5. The method of claim 1, wherein said exogenous polynucleotide encodes a miRNA or a precursor thereof.

6. The method of claim 1, wherein said exogenous polynucleotide encodes a siRNA or a precursor thereof.

7. The method of claim 1, wherein said exogenous polynucleotide is selected from the group consisting of SEQ ID NO: 10, 6-9, 21, 22, 23-37, 38-52, 1209, 1211, 1212.

8-16. (canceled)

17. A method of improving nitrogen use efficiency, abiotic stress tolerance, biomass, vigor or yield of a plant, the method comprising expressing within the plant an exogenous polynucleotide which downregulates an activity or expression of a gene encoding an RNAi molecule having a nucleic acid sequence at least 90% identical to SEQ ID NOs: 4, 1-3,5,57-449, 454-846 and 53-56, 1209, thereby improving nitrogen use efficiency, abiotic stress tolerance, biomass, vigor or yield of a plant.

18-19. (canceled)

20. The method of claim 17, wherein said polynucleotide encodes a miRNA-Resistant Target as set forth in SEQ ID N01104-1124.

21. The method of claim 17, wherein said isolated polynucleotide encodes a target mimic as set forth in SEQ ID NO: 18 or 19.

22-26. (canceled)

27. The method of claim 1, further comprising growing the plant under abiotic stress.

28. The method of claim 27, wherein said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

29-30. (canceled)

31. A method of improving nitrogen use efficiency, abiotic stress tolerance, biomass, vigor or yield of a plant, the method comprising expressing within the plant an exogenous polynucleotide encoding a polypeptide having an amino acid sequence at least 80% homologous to SEQ ID NOs: 927-1021, wherein said polypeptide is capable of regulating nitrogen use efficiency of the plant, thereby improving nitrogen use efficiency, abiotic stress tolerance, biomass, vigor or yield of the plant.

32. A transgenic plant exogenously expressing a polynucleotide encoding a polypeptide having an amino acid sequence at least 80% homologous to SEQ ID NOs: 927-1021, wherein said polypeptide is capable of regulating nitrogen use efficiency of the plant.

33. (canceled)

34. The method of claim 31, wherein said polynucleotide is selected from the group consisting of SEQ ID NO: 1022-1090.

35. The method of claim 31, wherein said polypeptide is selected from the group consisting of SEQ ID NO: 927-1021.

36-38. (canceled)

39. The method of claim 31, further comprising growing the plant under limiting nitrogen conditions.

40. The method of claim 31, further comprising growing the plant under abiotic stress.

41. The method of claim 40, wherein said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

42. The method of claim 31, wherein the plant is a monocotyledon.

43. The method of claim 31, wherein the plant is a dicotyledon.

44. A method of improving nitrogen use efficiency, abiotic stress tolerance, biomass, vigor or yield of a plant, the method comprising expressing within the plant an exogenous polynucleotide which downregulates an activity or expression of a polypeptide having an amino acid sequence at least 80% homologous to SEQ ID NOs: 854-894, wherein said polypeptide is capable of regulating nitrogen use efficiency of the plant, thereby improving nitrogen use efficiency, abiotic stress tolerance, biomass, vigor or yield of the plant.

45-50. (canceled)
